(19) United States
(12) Patent Application Publication
(10) Pub. No.: US 2026/0308223 A1
(43) Pub. Date: Oct. 8, 2026

(54) METHOD FOR GENETICALLY ENCODING 2-AMINOISOBUTYRIC ACID AND ITS ANALOGS AND APPLICATIONS THEREOF

(71) Applicants: Shaoxing Institute, Zhejiang University, Shaoxing (CN); Edipro Pharmaceutical Technology Inc., Hangzhou (CN)

(72) Inventors: Shixian LIN, Shaoxing City (CN); Wenlong DING, Shaoxing City (CN); Yongzhao XIONG, Shaoxing City (CN); Zhijian YAN, Shaoxing City (CN); Linzhen HU, Hangzhou City (CN)

(73) Assignees: Shaoxing Institute, Zhejiang University, Shaoxing (CN); Edipro Pharmaceutical Technology Inc., Hangzhou (CN)

(21) Appl. No.: 19/457,273

(22) Filed: Jan. 23, 2026

(30) Foreign Application Priority Data
Jan. 24, 2025 (CN) 202510121071.5

(51) Int. Cl. C07K 14/605; C12N 9/00; C12P 21/02

(52) U.S. Cl. CPC C07K 14/605; C12N 9/93; C12P 21/02; C07K 2319/85; C12Y 601/01007; C12Y 601/01026

(57) ABSTRACT

The present application relates to a noncanonical amino acid genetic encoding system, comprising a chimeric alanyl-tRNA synthetase (chAlaRS) or a nucleotide sequence encoding the chimeric alanyl-tRNA synthetase. The chimeric alanyl-tRNA synthetase comprises a tRNA binding domain (TD) and a catalytic domain (CD), wherein the tRNA binding domain (TD) and the catalytic domain (CD) of the chimeric alanyl-tRNA synthetase are derived from different aminoacyl-tRNA synthetases. The non-natural amino acid synthesis system is capable of introducing 2-aminoisobutyric acid (Aib) and analogs thereof at specific sites in proteins or polypeptides.

### CROSS REFERENCE TO RELATED APPLICATIONS

This application claims priority to Chinese Patent Application No. 202510121071.5 filed on Jan. 24, 2025, the entire disclosures of which are incorporated herein by reference.

### INCORPORATION BY REFERENCE OF SEQUENCE LISTING

The instant application contains a Sequence Listing which has been filed electronically in xml format and is hereby incorporated by reference in its entirety. Said xml copy, created on May 27, 2026, is named Q316730secondsubstitutesequencelisting.xml and is 250,970 bytes in size.

### FIELD OF THE INVENTION

The present application relates to the field of biomedicine, and involves a method for producing proteins comprising noncanonical amino acids. Specifically, it relates to a method for encoding proteins or polypeptides comprising one or more 2-aminoisobutyric acid (Aib) residues and analogs thereof, and the present application also relates to a system for expressing proteins or polypeptides comprising noncanonical amino acids using the aforementioned method.

### BACKGROUND OF THE INVENTION

Nature has selected a conserved set of amino acids, i.e., natural amino acids, to synthesize proteins with various biological functions, enriching their structure and function by changing the sequence of natural amino acids in proteins to synthesize proteins with various biological functions. In recent years, the biomedical field has pursued proteins with greater medicinal and economic value. To achieve the goal of protein functional innovation, researchers have considered utilizing the special functional groups and structural features of nonconical amino acids to endow target proteins with new physicochemical properties.

2-aminoisobutyric acid is a nitrogen-containing compound with various mechanisms of action. 2-aminoisobutyric acid is often introduced into protein and polypeptide drugs to resist hydrolase degradation and improve drug half-life and efficacy. Currently, most protein and polypeptide drugs containing 2-aminoisobutyric acid rely on chemical synthesis, which limits the types of proteins that can be synthesized, and the protein structures are relatively simple. In the field of biosynthesis, some studies have used mutants of alanine synthetase from extreme thermophilic archaea to genetically encode proteins or polypeptide drugs containing 2-aminoisobutyric acid. Although this solves the production of drugs with complex structures, the corresponding drug production efficiency, fidelity, and purity fail to meet demands.

Therefore, there is an urgent need for an efficient, high-fidelity, and high-purity biological synthesis method for proteins or polypeptides containing 2-aminoisobutyric acid and its analogs.

### SUMMARY OF THE INVENTION

The present application provides a method for synthesizing proteins or polypeptides containing 2-aminoisobutyric acid and its analogs using a biological pathway, comprising using a noncanonical amino acid genetic encoding system. The system comprises a chimeric alanyl-tRNA synthetase (chAlaRS) or a nucleotide sequence encoding the chimeric alanyl-tRNA synthetase, achieving the biosynthesis of 2-aminoisobutyric acid proteins or polypeptides in a host cell. At the same time, the method for synthesizing 2-aminoisobutyric acid proteins or polypeptides achieves significant improvements in production efficiency, fidelity, and/or purity.

The present application provides a noncanonical amino acid genetic encoding system comprising a chimeric alanyl-tRNA synthetase (chAlaRS) or a nucleotide sequence encoding the chimeric alanyl-tRNA synthetase, wherein the chimeric alanyl-tRNA synthetase comprises a tRNA binding domain (TD) and a catalytic domain (CD), and the tRNA binding domain (TD) and the catalytic domain (CD) of the chimeric alanyl-tRNA synthetase are derived from different aminoacyl-tRNA synthetases.

In some embodiments, the tRNA binding domain of the chimeric alanyl-tRNA synthetase is derived from a pyrrolysyl-tRNA synthetase (PylRS). In some embodiments, the catalytic domain of the chimeric alanyl-tRNA synthetase is derived from an alanyl-tRNA synthetase (AlaRS). In some embodiments, the aminoacyl-tRNA synthetase is derived from eubacteria or archaea, or is a variant of an enzyme derived from eubacteria or archaea.

In some embodiments, the pyrrolysyl-tRNA synthetase comprises MaPylRS, MbPylRS, MmPylRS, G1PylRS, Lum1PylRS, IR26PylRS, IntPylRS, NitraPylRS, or DebPylRS. In some embodiments, the pyrrolysyl-tRNA synthetase is MbPylRS and/or MmPylRS. In some embodiments, the pyrrolysyl-tRNA synthetase has an IPYE mutation compared to the wild type. In some embodiments, the tRNA binding domain (TD) comprises the sequence as shown in any one of SEQ ID NOs: 1-15, 86-87, or a variant thereof.

In some embodiments, the alanyl-tRNA synthetase is derived from Escherichia coli. In some embodiments, the catalytic domain (CD) comprises the sequence as shown in any one of SEQ ID NOs: 16-25, 88-111, or a variant thereof. In some embodiments, the chimeric alanyl-tRNA synthetase comprises the sequence as shown in any one of SEQ ID NOs: 26-35, 56-75, or a variant thereof.

In some embodiments, the noncanonical amino acid genetic encoding system further comprises a recombinant tRNA or a nucleotide sequence encoding the recombinant tRNA, wherein the recombinant tRNA comprises a first portion and a second portion, wherein the first portion matches the tRNA binding domain, and the second portion matches the catalytic domain (CD). In some embodiments, the first portion matches MbPylRS and/or MmPylRS. In some embodiments, the second portion matches AlaRS. In some embodiments, the second portion has an A-U mutation compared to the wild type. In some embodiments, the recombinant tRNA comprises the sequence as shown in any one of SEQ ID NOs: 36-49, or a variant thereof.

In some embodiments, the noncanonical amino acid genetic encoding system further comprises a host cell, wherein the host cell is a prokaryotic cell or a eukaryotic cell. In some embodiments, the synthesis system further comprises a transcription template or a nucleotide sequence encoding the transcription template, wherein the transcription template contains a codon encoding the noncanonical amino acid, and the anticodon of recombinant tRNA is reverse complementary to the codon encoding the non-natural amino acid.

In another aspect, the present application provides a method for preparing a compound, comprising the step of: a) preparing a polypeptide containing one or more Aib or analog amino acid residues using the synthesis system described above.

In some embodiments, the Aib or analog has the following structure:

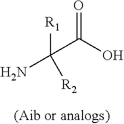

wherein R1 and/or R2 are H, C₁₋₅ alkyl, C₁₋₅—OH, or C₁₋₅—SH. In some embodiments, R1 and/or R2 are methyl.

In some embodiments, the polypeptide is a GLP-1 analog. In some embodiments, the polypeptide comprises as shown in any one of SEQ ID NOs: 76-80 and 112-117. The present application also provides the use of the synthesis system in preparing a polypeptide containing one or more Aib or its analogs

### DESCRIPTION OF THE DRAWINGS

The specific features of the invention involved in the present application are as shown in the appended claims. The features and advantages of the invention involved in the present application can be better understood by referring to the exemplary embodiments detailed below and the accompanying drawings. The brief description of the drawings is as follows:

FIG. 1 shows the construction of the chimeric alanyl-tRNA synthetase/tRNA system. (A) Construction of chimeric tRNA, the sequence of the left tRNA is GGAGCTATGATCATGTAGATCGAACGGACTCTAAATCCGTTCAGCCGGGTT AGATTCCCGGTAGCTCCACCA (SEQ ID NO 154), the sequence of the right tRNA is GGactcgTGATCATGTAGATCGAACGGACTCTAAATCCGTTCAGCCGGGTTAG ATTCCCGGcgagTCCACCA (SEQ ID NO 155); (B) Construction of chimeric alanyl-tRNA synthetase; (C) GFP amber codon suppression assay determining the activity of different constructs; (D) 3D structure simulation of chimeric alanyl-tRNA synthetase/tRNA.

FIG. 2 shows the schematic and results of screening chimeric alanyl-tRNA synthetase mutants recognizing Aib described in the present application. (A) Selection of mutation sites; (B) Activity of screened mutants; (C) LC-MS showing the fidelity of Aib recognition by screened mutants.

FIG. 3 shows the conservation of the amino acid binding pocket of alanyl-tRNA synthetase. HMM logos generated by homology alignment of 10,000 alanyl-tRNA synthetases from different species show the degree of conservation of corresponding amino acids.

FIG. 4 shows that directed evolution of relevant amino acids of chimeric alanyl-tRNA synthetase recognizing the A3-U70 pair on tRNA enhances the genetic encoding efficiency of Aib. (A) the simulation structure of chAlaRS/chAlaT with the A3-U70 pair colored with red, the residues for binding A3-U70 pair were also shown. (B) the workflow for selecting the high efficiency mutants. (C) the gfp amber suppression efficiency of variants obtained from the selection.

FIG. 5 shows the conservation of amino acid sites related to recognizing the A3-U70 pair. HMM logos generated by homology alignment of 10,000 alanyl-tRNA synthetases from different species show the degree of conservation of corresponding amino acids.

FIG. 6 shows the fidelity of Aib incorporation into various proteins.

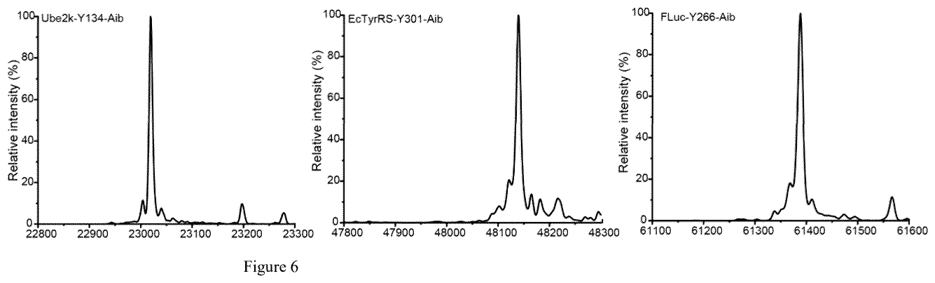

FIG. 7 shows the recognition of D-amino acids by the chimeric alanyl-tRNA synthetase/tRNA system. (A) ncAAs used in the experiment; (B) GFP amber suppression efficiency of different ncAAs; (C) LC-MS of three ncAAs showing high fidelity of incorporation.

FIG. 8 shows the recombinant expression to obtain GLP1 backbones containing different non-natural amino acids and their lipidated peptides. (A) Flowchart of recombinant expression of the backbone; (B) Coomassie brilliant blue staining showing purity; (C) LC-MS showing fidelity at each step.

FIG. 9 shows the efficacy of recombinantly expressed GLP1 polypeptides. (A) Resistance to DPP4 hydrolysis of GLP1 with different ncAAs introduced; (B) Effect of GLP1 with different ncAAs in glucose tolerance test; (C) Weight loss results in lean mice for GLP1 with different ncAAs; (D) Weight loss effect in DIO mice for GLP1 with different ncAAs.

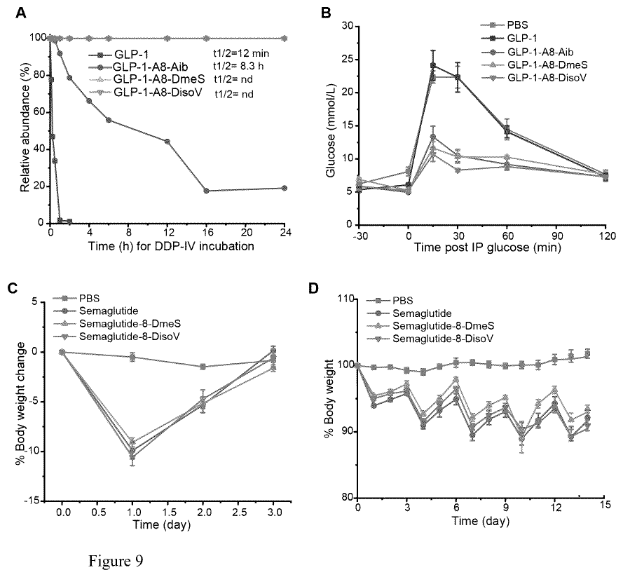

FIG. 10 shows the recombinant expression of the Tirzepatide backbone.

FIG. 11 shows the genetic encoding efficiency (A) and fidelity (B) of D-methyl-amino acids in mammalian cells.

FIG. 12 shows the recombinant expression of the GLP-1/GCCG receptor dual agonist Maz backbone.

FIG. 13 shows the recombinant expression of the GLP-1/GIP-1/GCCG receptor tri-agonist Pep-1 backbone.

FIG. 14 shows the recombinant expression of the GLP-1/Amylin receptor dual agonist amycretin backbone.

FIG. 15 shows the recombinant expression of the GLP-1/GIP-1/Amylin receptor tri-agonist P30 backbone.

### DEFINITION OF TERMS

The term “aminoacyl-tRNA synthetase” refers to an enzyme capable of binding a corresponding free amino acid and a corresponding free tRNA, and consuming ATP to catalyze the aminoacylation of the tRNA it binds with the amino acid it carries. The process of aminoacylation can occur intracellularly or in any reaction venue, such as a cell-free protein synthesis system. The corresponding amino acid can be one of the 20 natural amino acids, a noncanonical amino acid (including but not limited to selenocysteine, pyrrolysine, or pyrroline-carboxy-lysine), or a modified amino acid and/or amino acid derivative or analog thereof. Typically, one aminoacyl-tRNA synthetase can only bind one type of free amino acid; for example, an aminoacyl-tRNA synthetase can bind one amino acid selected from the group including but not limited to: alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, valine, selenocysteine, pyrrolysine, pyrroline-carboxy-lysine, or Aib. An aminoacyl-tRNA synthetase can bind one or more aminoacyl-tRNAs with different anticodons; for instance, an alanyl-tRNA synthetase can bind and aminoacylate multiple alanyl-tRNAs with different anticodons, wherein said alanyl-tRNA may correspond to the codons GCU, GCC, GCA, or GCG. In some embodiments, said aminoacyl-tRNA synthetase and tRNA can be endogenous, i.e., synthesized by the host cell without any genetic manipulation. In some embodiments, said aminoacyl-tRNA synthetase and tRNA can be exogenous, i.e., transformed by an exogenous nucleic acid molecule.

The term “catalytic domain (CD)” refers to a functional domain on an aminoacyl-tRNA synthetase capable of catalyzing the aminoacylation of the tRNA bound by the enzyme with the amino acid carried by the enzyme. Typically, the catalytic domain (CD) comprises a binding domain and/or an active center. The binding domain can recognize and bind tRNA, such as recognizing and binding the acceptor arm of the tRNA. Said active center can ligate the corresponding amino acid to the 3′-hydroxyl group of the tRNA via an esterification reaction, thereby achieving aminoacylation of the tRNA.

The term “tRNA binding domain (TD)” refers to a structural domain in an aminoacyl-tRNA synthetase capable of recognizing and binding a corresponding free tRNA. The tRNA binding domain (TD) binds free tRNA containing a specific enzyme binding site by recognizing said enzyme binding site on the tRNA. For example, for an alanine tRNA, the G3·U70 base pair on the acceptor arm is the site recognized by the tRNA binding domain; the alanyl-tRNA synthetase can correctly bind the alanyl-tRNA and complete aminoacylation by recognizing the G3·U70 base pair on the acceptor arm of the alanyl-tRNA, rather than aminoacylating other tRNAs, such as seryl-tRNA.

The term “alanyl-tRNA synthetase” refers to an endogenous tRNA synthetase, i.e., naturally expressed by the host cell without any genetic manipulation, which can bind alanine and alanyl-tRNA in the host cell and catalyze the aminoacylation of alanyl-tRNA with alanine. The alanyl-tRNA synthetase can bind and aminoacylate one or more alanyl-tRNAs corresponding to different codons, for example, said alanyl-tRNA can bind to codons GCU, GCC, GCA, or GCG. The alanyl-tRNA synthetase can be derived from eukaryotic cells or prokaryotic cells; for example, the alanyl-tRNA synthetase can be derived from Escherichia coli.

The term “pyrrolysyl-tRNA synthetase” refers to a class of orthogonal aminoacyl-tRNA synthetases, i.e., the pyrrolysyl-tRNA synthetase (PylRS) is orthogonal to the corresponding pyrrolysyl-tRNA (PylT). Said orthogonality means that compared to aminoacylating the corresponding pyrrolysyl-tRNA, the pyrrolysyl-tRNA synthetase aminoacylates other tRNAs with lower efficiency or cannot aminoacylate other tRNAs. Depending on the source, the pyrrolysyl-tRNA synthetase may include but is not limited to: pyrrolysyl-tRNA synthetase derived from Methanosarcina mazei (abbreviated as Mm, denoted as MmPylRS), pyrrolysyl-tRNA synthetase derived from Methanosarcina barkeri (abbreviated as Mb, denoted as MbPylRS), pyrrolysyl-tRNA synthetase derived from Methanomethylophilus alvus (abbreviated as Ma, denoted as MaPylRS), pyrrolysyl-tRNA synthetase derived from Methanogenic archaeon ISO4-G1 (abbreviated as G1, denoted as G1PylRS), pyrrolysyl-tRNA synthetase derived from Methanomassiliicoccus luminyensis (abbreviated as Lum1, denoted as Lum1PylRS), pyrrolysyl-tRNA synthetase derived from Candidatus Methanomethylophilus sp. 1R26 (abbreviated as 1R26, denoted as 1R26PylRS), pyrrolysyl-tRNA synthetase derived from Candidatus Methanomassiliicoccus intestinalis (abbreviated as Int, denoted as IntPylRS), pyrrolysyl-tRNA synthetase derived from Candidatus Nitrososphaeria archaeon (abbreviated as Nitra, denoted as NitraPylRS), pyrrolysyl-tRNA synthetase derived from Deltaproteobacteria bacterium (abbreviated as Deb, denoted as DebPylRS), as well as mutants, functional fragments, fusion enzymes, or chimeric enzymes of the aforementioned enzymes.

The term “chimeric alanyl-tRNA synthetase” refers to a modified enzyme based on an alanyl-tRNA synthetase, in which domains of the alanyl-tRNA synthetase are deleted, fused with, and/or replaced by domains from any other aminoacyl-tRNA synthetase, while retaining partial functional domains of the alanyl-tRNA synthetase. A chimeric alanyl-tRNA synthetase may replace any one or more domains of the alanyl-tRNA synthetase with any one or more domains of another aminoacyl-tRNA synthetase; for example, the catalytic domain (CD) or tRNA binding domain (TD) of the alanyl-tRNA synthetase can be replaced with any domain of another aminoacyl-tRNA synthetase. In some embodiments, the chimeric alanyl-tRNA synthetase may be orthogonal to the corresponding tRNA. In some embodiments, the tRNA binding domain (TD) of a pyrrolysyl-tRNA synthetase can be used to replace the tRNA binding domain (TD) of an alanyl-tRNA synthetase, wherein the C-terminus of the tRNA binding domain (TD) of the pyrrolysyl-tRNA synthetase can be fused to the N-terminus of the catalytic domain (CD) of the alanyl-tRNA synthetase, or the N-terminus of the tRNA binding domain (TD) of the pyrrolysyl-tRNA synthetase can be fused to the C-terminus of the catalytic domain (CD) of the alanyl-tRNA synthetase. There may be a connecting portion between the various domains of said chimeric alanyl-tRNA synthetase, and said connecting portion may be a linker peptide.

The term “variant” refers to a molecule similar to a specified molecule, wherein the variant is similar in function to the specified molecule before alteration but has changes in sequence, modification, and/or structure. Said specified molecule and/or variant can be a component of a translation system, such as an aminoacyl-tRNA synthetase or tRNA. Translation components refer to a collection of various components capable of incorporating amino acids into growing proteins, polypeptides, and/or amino acid residues. Components of a translation system may include but are not limited to, for example, ribosomes, tRNA, aminoacyl-tRNA synthetases, transcription templates, and/or amino acids. The sequence of a variant may include, but is not limited to, one sequence site variation, two sequence site variations, three sequence site variations, four sequence site variations, or five or more sequence site variations relative to the molecule before alteration, provided that the variant can perform the function of the molecule before alteration. For example, a variant pyrrolysyl-tRNA synthetase having an IPYE mutation (V31I/T56P/H62Y/A100E) compared to its wild type before alteration can also utilize an amino acid to aminoacylate the tRNA corresponding to the wild-type pyrrolysyl-tRNA synthetase. Modifications of a variant may include, but are not limited to, one modification variation, two modification variations, three modification variations, four modification variations, or five or more modification variations relative to the molecule before alteration, provided that the variant can perform the function of the molecule before alteration.

The term “recombinant tRNA” refers to a tRNA that can be expressed by a host cell through genetic manipulation. The recombinant tRNA can recognize codon on a transcription template and can be recognized and bound by a chimeric alanyl-tRNA synthetase, incorporating the amino acid carried by the chimeric alanyl-tRNA synthetase into a target protein or polypeptide. The recombinant tRNA can be preferentially aminoacylated by the chimeric alanyl-tRNA synthetase carrying an amino acid; that is, the chimeric alanyl-tRNA synthetase aminoacylates the recombinant tRNA with higher efficiency compared to aminoacylating tRNAs other than the recombinant tRNA. In some embodiments, the chimeric alanyl-tRNA synthetase may be completely unable to aminoacylate tRNAs other than the recombinant tRNA. Said recombinant tRNA can be chimeric, i.e., the structure of the recombinant tRNA is derived from two different tRNAs. In some embodiments, the acceptor arm of the recombinant tRNA can be derived from an alanyl-tRNA, and other structures excluding the acceptor arm can be derived from a pyrrolysyl-tRNA. Said recombinant tRNA can be a tRNA homologous to a tRNA derived from any species. Said homology refers to molecules with different sequences and/or modifications but similar functions. Said homology can be a sequence homology of at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%. The recombinant tRNA described herein can be a variant of a recombinant tRNA derived from any species. The recombinant tRNA can be in a loaded state with an amino acid or in an unloaded state without an amino acid. The amino acid loaded on the recombinant tRNA can be any amino acid, including but not limited to any natural amino acid or non-natural amino acid. In some embodiments, the amino acid loaded on the recombinant tRNA can be an Aib-class amino acid, and the recombinant tRNA can incorporate the loaded Aib-class amino acid into the target protein or polypeptide.

The term “first portion” refers to the part on the recombinant tRNA that binds to the tRNA binding domain (TD) on the aminoacyl-tRNA synthetase. The first portion of the recombinant tRNA includes a recognition element for the aminoacyl-tRNA synthetase, and said recognition element can consist of one or more nucleotides or one or more base pairs. The aminoacyl-tRNA synthetase achieves binding to the recombinant tRNA by recognizing the recognition element on the first portion via the tRNA binding domain. The recognition elements of different aminoacyl-tRNAs can be located at different parts of the tRNA; for example, for an alanine tRNA, the G3·U70 or A3:U70 base pair on the acceptor arm is the recognition element for the alanyl-tRNA synthetase to aminoacylate the alanyl-tRNA. An aminoacyl-tRNA can have one or more identical recognition elements, or multiple different recognition elements. The recognition element can be located in the anticodon loop region, anticodon arm, anticodon, acceptor arm, D-loop, D-arm, TY′C arm, TY′C loop, and/or variable loop of the tRNA. The first portion of the recombinant tRNA can include but is not limited to one or more of the following parts: anticodon loop region, anticodon arm, anticodon, acceptor arm, D-loop, D-arm, TY′C arm, TY′C loop, or variable loop.

The term “second portion” refers to the region on the recombinant tRNA capable of undergoing an aminoacylation reaction with an amino acid, said aminoacylation reaction being completed under the catalysis of the catalytic domain (CD) of the aminoacyl-tRNA synthetase. The second portion of said recombinant tRNA can be the acceptor arm of the tRNA, said acceptor arm comprising the CCA-OH at the 3′ end of the recombinant tRNA molecule. For example, under the catalysis of the aminoacyl-tRNA synthetase, the carried amino acid molecule is linked to the hydroxyl group at the 3′ end of the recombinant tRNA molecule via an esterification reaction to form an aminoacyl-tRNA.

The term “matching” (or “match”) refers to a process in which biological molecules interact with each other, or biological molecules interact with non-biological molecules, through domains, sequences, and/or groups, and finally complete a specific biological reaction. In some embodiments, matching between molecules allows two molecules to bind together through covalent bonds, ionic bonds, hydrogen bonds, and/or intermolecular forces; for example, complementary pairing between nucleotide bases via hydrogen bonds. In some embodiments, matching between molecules allows two molecules and/or other molecules to complete one or more specific chemical reactions including but not limited to the following group: substitution reaction, addition reaction, polymerization reaction, elimination reaction, esterification reaction, redox reaction, and decomposition reaction. For example, when the matching occurs between an aminoacyl-tRNA synthetase and a tRNA, the amino acid on the aminoacyl-tRNA synthetase and the tRNA undergo an esterification reaction.

The term “host cell” typically refers to a cell capable of being transformed by a nucleic acid molecule. When said nucleic acid molecule contains a gene of interest, the transformed host cell can express the transformed gene of interest. The term also includes progeny cells of said host cell, regardless of whether said progeny cells are completely identical in morphology or genetic structure to the original host cell, provided that the progeny cells contain the transformed nucleic acid molecule, they are still included within the scope of host cells in the present application. Host cells can include host cells existing in an isolated form, such as host cells in culture. Said host cell can also include a transcription template or a nucleic acid encoding a transcription template. Said host cell can be a eukaryotic cell or a prokaryotic cell. Prokaryotic cells refer to cells of organisms in the domain Eubacteria and domain Archaea. In some embodiments, Eubacteria and Archaea can be distinguished by morphological and biochemical methods, such as differences in ribosomal RNA sequences or RNA polymerase structures, the presence or absence of introns, sensitivity to different antibiotics, the presence or absence of cell wall peptidoglycan and other cell wall components, the presence or absence of branched or unbranched membrane lipid structures, and the presence or absence of histones and histone-like proteins. Examples of Eubacteria described herein include but are not limited to one or more of Escherichia coli, Thermus thermophilus, Bacillus subtilis, and Bacillus stearothermophilus. Examples of Archaea described herein include but are not limited to one or more of Methanococcus jannaschii (Mj), Methanosarcina mazei (Mm), Methanosarcina barkeri (Mb), Methanomethylophilus alvus (Ma), Methanogenic archaeon ISO4-G1 (G1), Methanomassiliicoccus luminyensis (Lum1), Candidatus methanomethylophilus sp. 1R26 (1R26), Candidatus Methanomassiliicoccus intestinalis (Int), Nitrososphaeria archaeon (Nitra), Deltaproteobacteria bacterium (Deb), Methanobacterium thermoautotrophicum (Mt), Methanococcus maripaludis, Methanopyrus kandleri, Halobacterium, Archaeoglobus fulgidus (Af), Pyrococcus furiosus (Pf), Pyrococcus horikoshii (Ph), Pyrobaculum aerophilum, Pyrococcus abyssi, Sulfolobus solfataricus (Ss), Sulfolobus tokodaii, Aeropyrum pernix (Ap), Thermoplasma acidophilum, Methanolobus psychrophilus R15 (Mp), Thermoproteus tenax (Tt), Thermococcus siculi (Ts), Psychrobacter arcticus 273-4 (Pa), and Thermoplasma volcanium. Eukaryotic cells refer to cells of organisms in the domain Eukaryota, such as yeast cells, mammalian cells, reptilian cells, avian cells, plant cells, algal cells, fungal cells, and/or insect cells, etc.

The term “transcription template” refers to a synthesis product of DNA transcription. A transcription template can include a primary transcription template of messenger RNA (mRNA) before processing, i.e., precursor messenger RNA (pre-mRNA), and/or a processed mature mRNA molecule. Said mature mRNA molecule is a mature functional form converted from precursor messenger RNA generated by DNA transcription and modified in several ways to produce different proteins and bioactive components. In some embodiments, the transcription template may include exons, introns, 5′ UTR and 3′ UTR, and any regulatory elements. In some embodiments, the transcription template may also include a modified transcription template, such as a spliced fusion protein or an exogenous nucleic acid sequence.

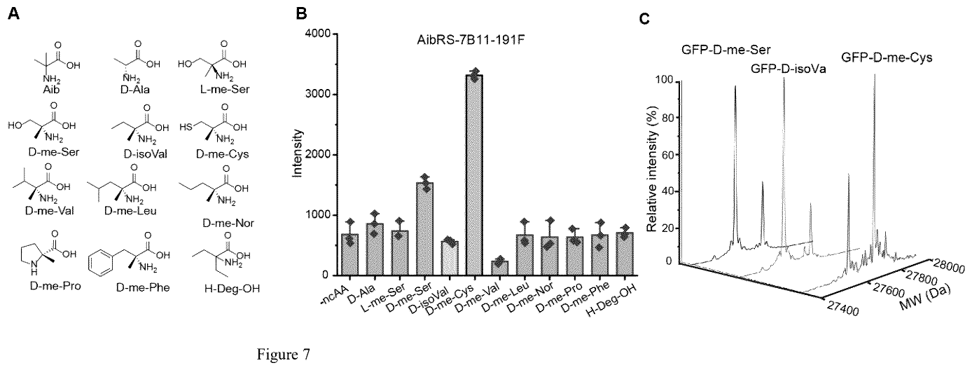

The term “Aib-class amino acid” refers to a general term for a class of amino acid compounds in which an amino group, a carboxyl group, and two alkyl substituents are present on the alpha carbon atom. Said alkyl groups can be any different C1-C5 alkyl groups, for example, one alkyl group can be any C5 alkyl group, and the other alkyl group can be any C4 alkyl group. Said alkyl groups can be any identical C1-C5 alkyl groups, such as both alkyl groups being methyl. Aib-class amino acids described herein include but are not limited to: AiB, dmeS (D-me-ser), dmeC (D-me-cys), D-isoVal. In some embodiments, said alkyl groups can both be methyl; for example, AIB can be 2-aminoisobutyric acid. The AIB-class amino acids in the present application can be as shown in (A) of FIG. 7.

The term “residue” refers to a specific amino acid in the backbone or side chain of a desired protein or polypeptide chain. One or more specific amino acid residues may exist on said protein or polypeptide chain, such as one, two, three, four, or five specific amino acid residues. One or more specific amino acids can exist at any position of the desired protein or polypeptide chain; for example, the specific amino acid can be on the main chain backbone or on a side chain. In some embodiments, the presence of a specific amino acid in said protein or polypeptide chain backbone or side chain refers to an AIB-class amino acid, including but not limited to one or more of the following group: AiB, dmeS, D-iso Val. For example, the specific amino acid can be 2-aminoisobutyric acid. In some embodiments, the protein or polypeptide chain can be a GLP-1 protein or an analog thereof; for example, it can be GLP-1.

### DETAILED DESCRIPTION

The present application provides a method for synthesizing proteins or polypeptides containing 2-aminoisobutyric acid (Aib) using a biological pathway, comprising using a noncaonical amino acid genetic encoding system which includes a chimeric alanyl-tRNA synthetase (chAlaRS) or a nucleotide sequence encoding the chimeric alanyl-tRNA synthetase, to achieve the biosynthesis of Aib-containing proteins or polypeptides in a host cell. Simultaneously, the method for synthesizing Aib-containing proteins or polypeptides achieves significant improvements in production efficiency, fidelity, and/or purity.

Compared with chemical synthesis methods, the synthesis method for Aib-containing proteins or polypeptides provided herein can achieve the synthesis of proteins with larger molecular weights or longer polypeptide chains without chemical side reactions during the synthesis process, such as oxidation and deamidation. Furthermore, the method of the present application can also achieve the synthesis of proteins or polypeptides with more complex structures, such as proteins with complex three-dimensional structures.

Current biological synthesis methods for Aib-containing proteins or polypeptides use mutants of alanyl-tRNA synthetase from extreme thermophilic archaea to genetically encode Aib proteins or polypeptides, but the corresponding production efficiency, fidelity, and protein purity are poor. The alanyl-tRNA synthetase of extreme thermophilic archaea is its endogenous aminoacyl-tRNA synthetase. Due to evolutionary differences, the extreme thermophilic archaeal alanyl-tRNA synthetase/alanyl-tRNA possesses a certain degree of orthogonality in other host cells. This technology enhances the orthogonality of the extreme thermophilic archaeal alanyl-tRNA synthetase by modifying its structure, allowing it to carry Aib. However, while the extreme thermophilic archaeal alanyl-tRNA synthetase/alanyl-tRNA encodes Aib, it still recognizes the host cell's endogenous alanyl-tRNA synthetase/alanyl-tRNA, leading to a large amount of alanine misincorporation, thereby resulting in extremely low production efficiency and fidelity. Moreover, this system is difficult to apply to eukaryotic organisms such as mammalian cells.

Studies have found that although the extreme thermophilic archaeal alanyl-tRNA synthetase/alanyl-tRNA possesses a certain degree of orthogonality in other host cells, as translation components derived for encoding natural amino acids, the orthogonality they possess in host cells is still insufficient to achieve efficient or high-fidelity production of Aib proteins or polypeptides. Therefore, there is a need for translation components encoding non-natural amino acids with stronger orthogonality to realize the production of Aib proteins or polypeptides.

The chimeric alanyl-tRNA synthetase (chAlaRS)/alanyl-tRNA (chAlaT) system provided by the present application possesses both orthogonality and the ability to efficiently encode 2-aminoisobutyric acid proteins or polypeptides. Unlike translation components from extreme thermophilic archaea, the translation components provided herein do not recognize, or recognize with very low probability, the endogenous aminoacyl-tRNA synthetase/tRNA, i.e., they possess high orthogonality. The tRNA binding domain (TD) and the catalytic domain (CD) of the chimeric alanyl-tRNA synthetase provided herein are derived from different aminoacyl-tRNA synthetases, wherein the tRNA binding domain is derived from pyrrolysyl-tRNA synthetase (PylRS) and the catalytic domain is derived from alanyl-tRNA synthetase (AlaRS). Said components show significant improvements in the production efficiency, fidelity, and purity of proteins or polypeptides containing Aib and its analogs.

Pyrrolysyl-tRNA synthetase (PylRS)/pyrrolysyl-tRNA (PylT) are naturally orthogonal cellular translation components. Therefore, when PylRS/PylT are introduced into a host cell to encode a non-natural amino acid, the probability of mismatching of the non-natural amino acid carried by the PylRS is extremely low; that is, it does not affect the normal physiological activities of the host cell and ensures the fidelity and synthesis efficiency of the non-natural amino acid protein synthesis. Thus, PylRS/PylT are generally considered an ideal pair for encoding non-natural amino acids in prokaryotes and eukaryotes.

However, PylRS/PylT only has high insertion activity for non-natural amino acids with specific structures, and PylRS/PylT from different sources (including but not limited to MaPylRS, MbPylRS, MmPylRS, G1PylRS, Lum1PylRS, 1R26PylRS, IntPylRS, NitraPylRS, and DebPylRS, etc.) have significant differences in insertion efficiency for different non-natural amino acids. Therefore, the effect of using a PylRS system or a chimeric PylRS system to synthesize Aib proteins or polypeptides is unpredictable.

Current research on the orthogonality of PylRS/PylT suggests that PylT has a very unique structure, lacking many conserved invariant structural elements present in typical tRNAs. Among all unique structural features, the short variable region, non-canonical T-loop, and small D-loop contribute to the orthogonality of PylT. Secondly, existing studies have also proven that the tRNA binding domain (TD) of PylRS folds into a compact globular structure, and the TD of PylRS binds to PylT by recognizing the special structure of PylT, thereby conferring orthogonality to the PylRS/PylT pair. Combining the above research results on PylRS/PylT orthogonality, it is currently very difficult to develop a chimeric aminoacyl-tRNA synthetase that retains PylRS/PylT orthogonality.

First, the sequence length of the tRNA binding domain (TD) and the protein size are difficult to determine. Second, the transplantation of domains and special tRNA structures makes it difficult to ensure the retention of orthogonality function. When the tRNA binding domain (TD) of PylRS is transplanted to other chimeric enzymes, due to the loss of other domains of PylRS itself and the influence of the new chimeric domains, it is difficult to predict whether the tRNA binding domain (TD) of PylRS can still fold into a compact globular structure and perform the function of recognizing PylT.

In summary, the effect of using a chimeric pyrrolysyl-tRNA synthetase system to synthesize Aib proteins or polypeptides is unpredictable. The synthesis system of the chimeric alanyl-tRNA synthetase (chAlaRS) provided by the present application creatively completes the chimera of pyrrolysyl-tRNA synthetase and alanyl-tRNA synthetase, enhancing the production efficiency, fidelity, and purity of Aib proteins or polypeptides while retaining high orthogonality.

The present application provides a non-natural amino acid synthesis system comprising a chimeric alanyl-tRNA synthetase (chAlaRS) or a nucleic acid encoding the chimeric alanyl-tRNA synthetase, wherein said chimeric alanyl-tRNA synthetase comprises a tRNA binding domain (TD) and a catalytic domain (CD), and wherein the tRNA binding domain (TD) and the catalytic domain (CD) of said chimeric alanyl-tRNA synthetase are derived from different aminoacyl-tRNA synthetases.

### Chimeric Alanyl-tRNA Synthetase

The present application provides a chimeric alanyl-tRNA synthetase (chAlaRS), comprising a tRNA binding domain (TD) and a catalytic domain (CD), wherein the tRNA binding domain (TD) and the catalytic domain (CD) of said chimeric alanyl-tRNA synthetase are derived from different aminoacyl-tRNA synthetases.

The chimeric alanyl-tRNA synthetase used in the present application can be chimeric with the tRNA binding domain (TD) of another aminoacyl-tRNA synthetase, thereby enabling said chimeric alanyl-tRNA synthetase to possess the function of recognizing tRNAs other than alanine tRNA.

The tRNA binding domain of said chimeric alanyl-tRNA synthetase can be derived from any orthogonal aminoacyl-tRNA synthetase; for example, it can be derived from a pyrrolysyl-tRNA synthetase. The tRNA binding domain of the pyrrolysyl-tRNA synthetase can recognize and bind pyrrolysyl-tRNA and any tRNA containing a pyrrolysyl-tRNA recognition element.

Specifically, the pyrrolysyl-tRNA synthetase can be one or more of the following enzymes: MmPylRS, MbPylRS, MaPylRS, G1PylRS, Lum1PylRS, 1R26PylRS, IntPylRS, NitraPylRS, DebPylRS, as well as mutants, functional fragments, fusion enzymes, or chimeric enzymes of the aforementioned enzymes.

The pyrrolysyl-tRNA synthetase can be MbPylRS and/or MmPylRS. In some embodiments, said pyrrolysyl-tRNA synthetase can be MbPylRS. In some embodiments, said pyrrolysyl-tRNA synthetase can be MmPylRS. Specifically, the pyrrolysyl-tRNA synthetase may have an IPYE mutation (V31I/T56P/H62Y/A100E) compared to its wild type.

In the chimeric alanyl-tRNA synthetase, said tRNA binding domain (TD) may comprise the sequence as shown in any one of SEQ ID NOs: 1-15, 86-87, or a variant thereof.

The chimeric alanyl-tRNA synthetase used in the present application can be chimeric with the catalytic domain (CD) of another aminoacyl-tRNA synthetase, thereby enabling said chimeric alanyl-tRNA synthetase to possess the function of catalyzing the aminoacylation of tRNAs other than alanine tRNA.

The chimeric alanyl-tRNA synthetase used in the present application can also be derived from alanyl-tRNA synthetase (AlaRS).

The alanyl-tRNA synthetase can be derived from eubacteria or archaea, or be a variant of an enzyme derived from eubacteria or archaea. The archaea can be one or more of Mj, Mm, Mb, Ma, G1, Lum1, 1R26, Int, Nitra, Deb, Mt, Methanococcus maripaludis, Methanopyrus kandleri, Halobacterium, Af, Pf, Ph, Pyrobaculum aerophilum, Pyrococcus abyssi, Ss, Sulfolobus tokodaii, Ap, Thermoplasma acidophilum, and Thermoplasma volcanium.

The eubacteria can be one of Escherichia coli, Thermus thermophilus, Bacillus subtilis, and Bacillus stearothermophilus.

The alanyl-tRNA synthetase can be derived from Escherichia coli.

The catalytic domain (CD) of said alanyl-tRNA synthetase comprises the sequence as shown in any one of SEQ ID NOs: 16-25, 88-111, or a variant thereof.

The tRNA binding domain (TD) of said chimeric alanyl-tRNA synthetase can be derived from a pyrrolysyl-tRNA synthetase, and the catalytic domain (CD) can be derived from an alanyl-tRNA synthetase.

The tRNA binding domain (TD) of said chimeric alanyl-tRNA synthetase can be derived from MbPylRS and/or MmPylRS, and the catalytic domain (CD) can be derived from an alanyl-tRNA synthetase of Escherichia coli. The N-terminus of the tRNA binding domain (TD) of MbPylRS and/or MmPylRS can be connected to the C-terminus of the catalytic domain (CD) of the Escherichia coli alanyl-tRNA synthetase. The C-terminus of the tRNA binding domain (TD) of MbPylRS and/or MmPylRS can be connected to the N-terminus of the catalytic domain (CD) of the Escherichia coli alanyl-tRNA synthetase.

The tRNA binding domain (TD) of said chimeric alanyl-tRNA synthetase can be derived from MbPylRS, and the catalytic domain (CD) can be derived from an alanyl-tRNA synthetase of Escherichia coli. The N-terminus of the tRNA binding domain (TD) of MbPylRS can be connected to the C-terminus of the catalytic domain (CD) of the Escherichia coli alanyl-tRNA synthetase. The C-terminus of the tRNA binding domain (TD) of MbPylRS can be connected to the N-terminus of the catalytic domain (CD) of the Escherichia coli alanyl-tRNA synthetase.

The tRNA binding domain (TD) of said chimeric alanyl-tRNA synthetase can be derived from MmPylRS, and the catalytic domain (CD) can be derived from an alanyl-tRNA synthetase of Escherichia coli. The N-terminus of the tRNA binding domain (TD) of MmPylRS can be connected to the C-terminus of the catalytic domain (CD) of the Escherichia coli alanyl-tRNA synthetase. The C-terminus of the tRNA binding domain (TD) of MmPylRS can be connected to the N-terminus of the catalytic domain (CD) of the Escherichia coli alanyl-tRNA synthetase.

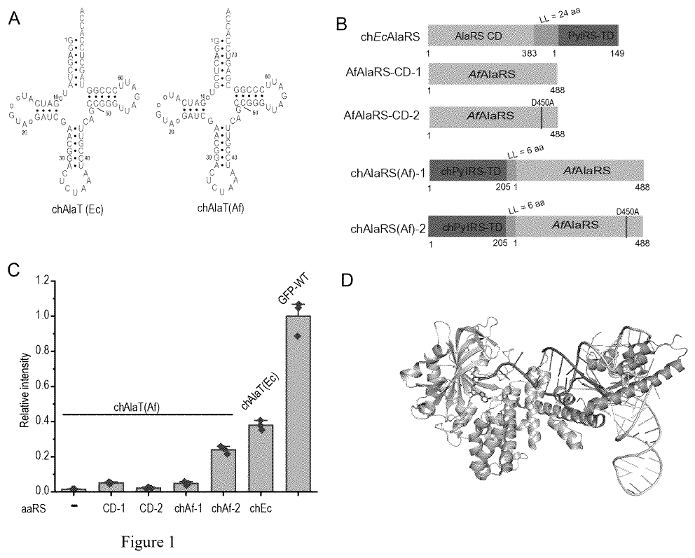

For example, as shown in (B) of FIG. 1, the CD region of the chimeric alanyl-tRNA synthetase chEcAlaRS is derived from Escherichia coli (SEQ ID: 16), and the TD region is derived from MbPylRS (SEQ ID: 12).

There may be a connecting molecule between the tRNA binding domain (TD) and the catalytic domain (CD) of said chimeric alanyl-tRNA synthetase, and said connecting molecule may be a linker peptide. Said linker peptide may comprise the sequence as shown in any one of SEQ ID NOs: 50-55, or a variant thereof.

The chimeric alanyl-tRNA synthetase may comprise the sequence as shown in any one of SEQ ID NOs: 26-35, 56-75, or a variant thereof.

The chimeric alanyl-tRNA synthetase may comprise one or more efficiency-enhancing mutations. For example, the chimeric alanyl-tRNA synthetase may comprise an IPYE mutation (V31I/T56P/H62Y/A100E) to improve the enzymatic efficiency of the chimeric alanyl-tRNA synthetase. For example, chPylRS, MmPylRS, or MbPylRS may further comprise an IPYE mutation (V31I/T56P/H62Y/A100E) compared to their wild types.

In some embodiments, said noncanonical amino acid is Aib.

The chimeric alanyl-tRNA synthetase has an amino acid recognition region in the catalytic domain (CD), wherein a mutation exists in said amino acid recognition region, and said amino acid recognition region specifically binds alanine in a wild-type alanyl-tRNA synthetase.

In the wild-type alanyl-tRNA synthetase of eubacteria or archaea, the sites recognizing alanine are highly conserved. Taking Archaeoglobus fulgidus (Af) as an example, W191, C200, T212, V214, D242, and T243 are related to alanine recognition, wherein the position numbering corresponds to the position in the wild-type alanyl-tRNA synthetase of Archaeoglobus fulgidus.

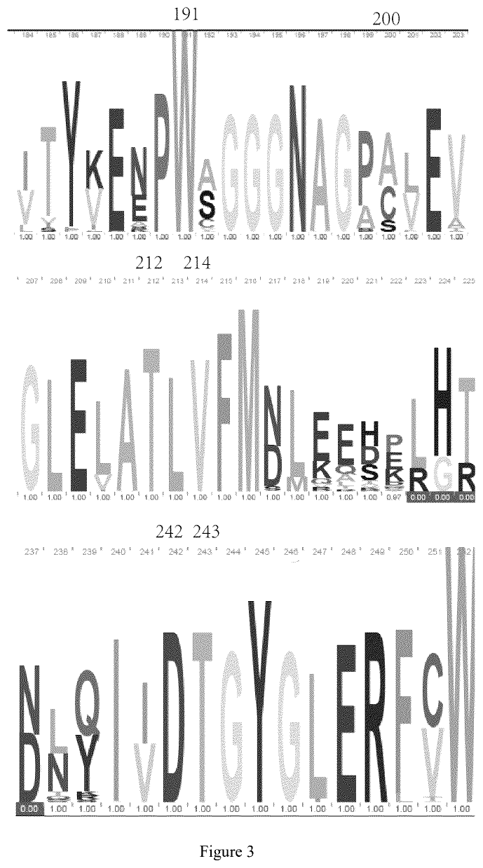

As shown in FIG. 3, homology alignment of more than 10,000 sequences using the Af sequence as a benchmark shows that the above positions are highly conserved among alanine recognition sites.

For example, comparing Af with Mp, W191, C200, T212, V214, D242, and T243 of Archaeoglobus fulgidus (Af) correspond to W189/C198/T210/V212/D240/T241 of Mp. Comparing Af with Tt, W191, C200, T212, V214, D242, and T243 of Af correspond to W187/S196/T208/V210/D230/T231 of Tt. Comparing Af with Ts, W191, C200, T212, V214, D242, and T243 of Af correspond to W189/A198/T210/V212/D245/T246 of Ts.

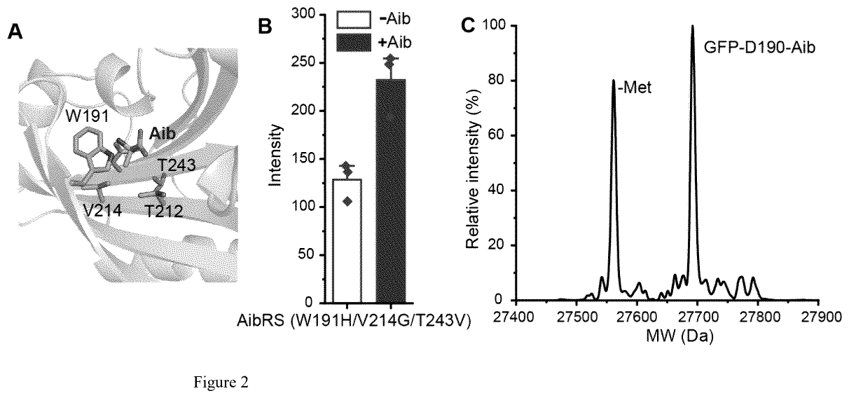

The above positions in their respective chimeric alanyl-tRNA synthetases are all related to the pocket structure binding alanine, as shown in FIG. 2.

The inventors found that performing a first class of mutations at the above sites can improve the synthesis efficiency of Aib-class amino acids. The positions of the first class of mutations are related to the pocket structure binding alanine. The following takes Af as an example.

For example, said chimeric alanyl-tRNA synthetase has a mutation in the amino acid recognition region, wherein said mutation is selected from a combination of one or more of the following sites: W191, C200, T212, V214, D242, and T243. For example, W191, C200, T212, V214, D242, or T243, and any combination thereof.

For example, said chimeric alanyl-tRNA synthetase has a mutation in the amino acid recognition region, wherein said mutation is selected from a combination of one or more of the following sites: W191F, W191H, W191L, W191I, W191Y, W191V, and W191M. For example, W191F, W191H, W191L, W191I, W191Y, W191V, or W191M, and any combination thereof.

For example, said chimeric alanyl-tRNA synthetase has a mutation in the amino acid recognition region, wherein said mutation is selected from a combination of one or more of the following sites: C200A, C200G, and C200V. For example, C200A, C200G, or C200V, and any combination thereof.

For example, said chimeric alanyl-tRNA synthetase has a mutation in the amino acid recognition region, wherein said mutation is selected from a combination of one or more of the following sites: T212S, T212A, and T212G. For example, T212S, T212A, or T212G, or any combination thereof.

For example, said chimeric alanyl-tRNA synthetase has a mutation in the amino acid recognition region, wherein said mutation is selected from a combination of one or more of the following sites: V214A and V214G. For example, V214A or V214G.

For example, said chimeric alanyl-tRNA synthetase has a mutation in the amino acid recognition region, wherein said mutation is selected from a combination of one or more of the following sites: D242E, D242S, and D242T. For example, D242E, D242S, or D242T, or any combination thereof.

For example, the chimeric alanyl-tRNA synthetase has a mutation in the amino acid recognition region, wherein said mutation is selected from a combination of one or more of the following sites: T243A, T243V, T243L, T243S, T243C, T243I, T243G, and T243F. For example, T243A, T243V, T243L, T243S, T243C, T243I, T243G, or T243F, and any combination thereof.

For example, said chimeric alanyl-tRNA synthetase has a mutation in the amino acid recognition region, wherein said mutation is selected from one or more of the following: W191F/V214G/T243V, W191F/V214G, W191F/V214G/T243S, W191F/V214G/T243C, W191F/V214G/T243L, W191F/V214G/T243I, W191F/V214G/T243F, W191F/V214G/T243A, W191Y/V214G/T243V, W191Y/V214G, W191Y/V214G/T243S, W191Y/V214G/T243C, W191Y/V214G/T243L, W191Y/V214G/T243I, W191Y/V214G/T243F, W191Y/V214G/T243A, W191L/V214G/T243V, W191L/V214G, W191L/V214G/T243S, W191L/V214G/T243C, W191L/V214G/T243L, W191L/V214G/T243I, W191L/V214G/T243F, W191L/V214G/T243A, V214G/T243V, V214G, V214G/T243S, V214G/T243C, V214G/T243L, V214G/T243I, V214G/T243F, V214G/T243A, W191H/V214G/T243V, W191H/V214G, W191H/V214G/T243S, W191H/V214G/T243C, W191H/V214G/T243L, W191H/V214G/T243I, W191H/V214G/T243F, W191H/V214G/T243A, W191F/V214A/T243V, W191F/V214A, W191F/V214A/T243S, W191F/V214A/T243C, W191F/V214A/T243L, W191F/V214A/T243I, W191F/V214A/T243F, W191F/V214A/T243A, W191F/V214G/D242S/T243V, W191H/V214G/D242S/T243V, W191Y/V214G/D242S/T243V, W191F/T212S/V214G/T243V, W191F/T212A/V214G/T243V, W191F/T212S/V214G/, W191H/T212S/V214G/T243V.

Those skilled in the art can adopt similar mutations at corresponding positions of other alanyl-tRNA synthetases according to the homology of Af to improve the synthesis efficiency of polypeptides containing Aib.

The chimeric alanyl-tRNA synthetase has a tRNA A3:U70 or G3:U70 recognition region on the catalytic domain (CD), wherein a mutation exists in said A3:U70 or G3:U70 recognition region, and said amino acid recognition region specifically binds the A3:U70 or G3:U70 recognition region of tRNA in wild-type alanyl-tRNA synthetase.

In the wild-type alanyl-tRNA synthetase of eubacteria or archaea, the sites recognizing A3:U70 or G3:U70 are highly conserved. Taking Archaeoglobus fulgidus (Af) as an example, N359, D450, and E457 are related to the sites recognizing A3:U70 or G3:U70, wherein the position numbering corresponds to the position in the wild-type alanyl-tRNA synthetase of Archaeoglobus fulgidus.

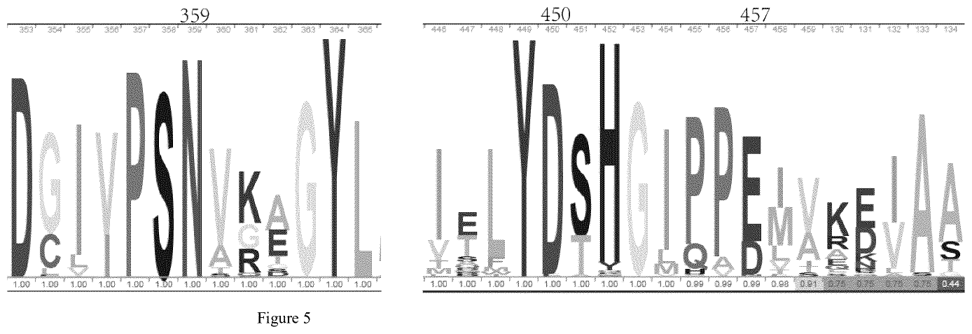

As shown in FIG. 5, homology alignment of more than 10,000 sequences using the Af sequence as a benchmark shows that the above positions are highly conserved among alanine recognition sites.

For example, comparing Af with Mp, N359, D450, and E457 of Archaeoglobus fulgidus (Af) correspond to N357/D454/E461 of Mp. Comparing Af with Tt, N359, D450, and E457 of Af correspond to N341/D437/E444 of Tt. Comparing Af with Ts, N359, D450, and E457 of Af correspond to N357/E456/E463 of Ts.

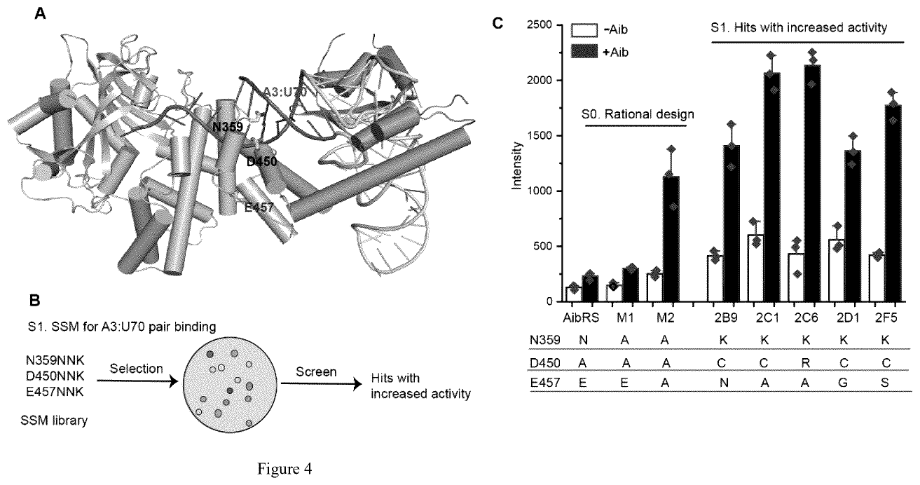

The above positions in their respective chimeric alanyl-tRNA synthetases are all related to the site binding A3:U70 or G3:U70, as shown in (A) of FIG. 4.

The inventors found that performing a second class of mutations at the above sites can improve the synthesis efficiency of Aib-class amino acids. The positions of the second class of mutations are related to binding A3:U70 or G3:U70. The following takes Af as an example.

For example, there is a mutation that enhances efficiency in the catalytic domain of the chimeric alanyl-tRNA synthetase, said mutation comprising N359K, N359A, or N359V. For example, there is a mutation that enhances efficiency in the catalytic domain of the chimeric alanyl-tRNA synthetase, said mutation comprising D450A, D450R, D450C, D450V, or D450M.

For example, there is a mutation that enhances efficiency in the catalytic domain of the chimeric alanyl-tRNA synthetase, said mutation comprising E457N, E457S, E457A, E457Q, E457M, or E457G.

Those skilled in the art can adopt similar mutations at corresponding positions of other alanyl-tRNA synthetases according to the homology of Af to improve the synthesis efficiency of polypeptides containing Aib.

Recombinant tRNA

Said non-natural amino acid synthesis system may further comprise a recombinant tRNA or a nucleic acid encoding the recombinant tRNA, wherein the recombinant tRNA comprises a first portion and a second portion, wherein the first portion matches the tRNA binding domain, and the second portion matches the catalytic domain (CD).

The first portion of said recombinant tRNA can match the tRNA binding domain on the aminoacyl-tRNA synthetase.

The first portion of the recombinant tRNA can include, but is not limited to, one or more of the following parts: anticodon loop region, anticodon loop arm, anticodon, acceptor arm, D-loop, D-arm, TY′C arm, TY′C loop, or variable loop.

The first portion of the recombinant tRNA can further include a recognition element for the aminoacyl-tRNA synthetase, and said recognition element can consist of one or more nucleotides or one or more base pairs. The aminoacyl-tRNA synthetase can bind to the recombinant tRNA by recognizing the recognition element on the first portion via the tRNA binding domain.

The recombinant tRNA can have one or more identical recognition elements, or multiple different recognition elements. The recognition elements of different recombinant tRNAs can be located at different parts of the tRNA. The recognition element can be located in the anticodon loop region, anticodon arm, anticodon, acceptor arm, D-loop, D-arm, TYPC arm, TY′C loop, and/or variable loop of the tRNA. For example, for an alanine tRNA, the G3·U70 or A3·U70 base pair on the acceptor arm is the recognition element for the alanyl-tRNA synthetase to aminoacylate the alanyl-tRNA. “G3·U70” means that for a tRNA, the 3rd position is G and the 70th position is U. “A3·U70” means that for a tRNA, the 3rd position is A and the 70th position is U.

The first portion of said recombinant tRNA can be derived from other tRNAs, such as pyrrolysyl-tRNA.

The first portion of said recombinant tRNA can match the tRNA binding domain on MbPylRS and/or MmPylRS. The first portion of said recombinant tRNA can be derived from MbPylT and/or MmPylT. The first portion of said recombinant tRNA can be derived from the anticodon loop region, anticodon loop arm, anticodon, acceptor arm, D-loop, D-arm, TP′C arm, TY′C loop, and/or variable loop of MbPylT and/or MmPylT.

The second portion of said recombinant tRNA is the region on the recombinant tRNA capable of undergoing an aminoacylation reaction with an amino acid, said aminoacylation reaction being completed under the catalysis of the catalytic domain (CD) of the aminoacyl-tRNA synthetase.

The second portion of said recombinant tRNA can be the acceptor arm of the tRNA, and said acceptor arm can include the CCA-OH at the 3′ end of the recombinant tRNA molecule. For example, under the catalysis of the aminoacyl-tRNA synthetase, the carried amino acid molecule is linked to the hydroxyl group at the 3′ end of the recombinant tRNA molecule via an esterification reaction to form an aminoacyl-tRNA.

The second portion of said recombinant tRNA can be derived from other tRNAs, such as the alanyl-tRNA of Escherichia coli. The second portion of said recombinant tRNA matches the alanyl-tRNA synthetase of Escherichia coli. The second portion of said recombinant tRNA can be derived from the acceptor arm of the alanyl-tRNA of Escherichia coli.

In some embodiments, the second portion of said recombinant tRNA has an A-U mutation compared to the wild-type Escherichia coli alanyl-tRNA.

The first portion and the second portion of said recombinant tRNA can be derived from different tRNAs. For example, the first portion of said recombinant tRNA can be derived from MbPylT and/or MmPylT, and the second portion of the recombinant tRNA can be derived from the alanyl-tRNA of Escherichia coli.

Said recombinant tRNA may comprise the sequence as shown in any one of SEQ ID NOs: 36-49, or a variant thereof. X1X2X3 represents the corresponding anticodon; for example, X1X2X3 can be CGA, UCA, or CUA.

### Host Cell and Transcription Template

The noncanonical amino acid genetic encoding system may further comprise a host cell, wherein said host cell can be a prokaryotic cell or a eukaryotic cell.

The prokaryotic cell can be Eubacteria and/or Archaea. Examples of Eubacteria can include, but are not limited to, one or more of Escherichia coli, Thermus thermophilus, Bacillus subtilis, and Bacillus stearothermophilus.

Examples of Archaea can include, but are not limited to, one or more of Methanococcus jannaschii (Mj), Methanosarcina mazei (Mm), Methanosarcina barkeri (Mb), Methanomethylophilus alvus (Ma), Methanogenic archaeon ISO4-G1 (G1), Methanomassiliicoccus luminyensis (Lum1), Candidatus Methanomethylophilus sp. 1R26 (1R26), Candidatus Methanomassiliicoccus intestinalis (Int), Nitrososphaeria archaeon (Nitra), Deltaproteobacteria bacterium (Deb), Methanobacterium thermoautotrophicum (Mt), Methanococcus maripaludis, Methanopyrus kandleri, Halobacterium, Archaeoglobus fulgidus (Af), Pyrococcus furiosus (Pf), Pyrococcus horikoshii (Ph), Pyrobaculum aerophilum, Pyrococcus abyssi, Sulfolobus solfataricus (Ss), Sulfolobus tokodaii, Aeropyrum pernix (Ap), Thermoplasma acidophilum, and Thermoplasma volcanium.

Eukaryotic cells can include, but are not limited to: yeast cells, mammalian cells, reptilian cells, avian cells, plant cells, algal cells, fungal cells, and/or insect cells.

In the present application, the host cell can be a eukaryotic cell, such as a yeast cell, mammalian cell, reptilian cell, avian cell, plant cell, algal cell, fungal cell, and/or insect cell, etc.

In said noncanonical amino acid genetic encoding system, the host cell further comprises a transcription template or a nucleic acid encoding the transcription template, wherein said transcription template contains a codon encoding the non-natural amino acid, and said recombinant tRNA is reverse complementary to the codon encoding the noncanonical amino acid.

The host cell can contain or express a transcription template, such as mRNA. Proteins and/or polypeptides containing the noncanonical amino acid are expressed based on the transcription template of the host cell.

The transcription template can be produced by the expression of an endogenous gene of the host cell. The transcription template can also be produced by the expression of an exogenous gene; for example, the transcription template can be introduced into the host cell with the help of a vector, allowing the transcription template to be expressed in the host cell.

### Methods and Uses

In another aspect, the present application also provides a method for producing a protein or polypeptide comprising one or more 2-aminoisobutyric acid and its analogs (Aib) in a host cell, said method comprising culturing the host cell with the following substances: a chimeric alanyl-tRNA synthetase (chAlaRS) or a nucleic acid encoding the chimeric alanyl-tRNA synthetase, and a recombinant tRNA or a nucleic acid encoding the chAlaT, wherein said chimeric alanyl-tRNA synthetase comprises a tRNA binding domain (TD) and a catalytic domain (CD), and wherein said tRNA binding domain (TD) and catalytic domain (CD) are derived from different aminoacyl-tRNA synthetases.

In said method, said AiB may have the following structure:

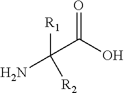

(Formula AiB), wherein R1 and/or R2 can be C₁₋₅ alkyl, wherein R1 and/or R2 can be methyl.

In said method, the protein or polypeptide can be a GLP-1 analog. In some embodiments, said polypeptide comprises the sequence as shown in any one of SEQ ID NOs: 76-85. The chimeric alanyl-tRNA synthetase used in the present application can be chimeric with the tRNA binding domain (TD) of another aminoacyl-tRNA synthetase, thereby enabling said chimeric alanyl-tRNA synthetase to possess the function of recognizing tRNAs other than alanine tRNA.

In said method, the tRNA binding domain of the chimeric alanyl-tRNA synthetase can be derived from any orthogonal aminoacyl-tRNA synthetase; for example, it can be derived from a pyrrolysyl-tRNA synthetase. The tRNA binding domain of the pyrrolysyl-RNA synthetase can recognize and bind pyrrolysyl-tRNA and any tRNA containing a pyrrolysyl-tRNA recognition element.

Specifically, the pyrrolysyl-tRNA synthetase can be one or more of the following enzymes: MmPylRS, MbPylRS, MaPylRS, G1PylRS, Lum1PylRS, 1R26PylRS, IntPylRS, NitraPylRS, DebPylRS, as well as mutants, functional fragments, fusion enzymes, or chimeric enzymes of the aforementioned enzymes.

The pyrrolysyl-tRNA synthetase can be MbPylRS and/or MmPylRS. In some embodiments, said pyrrolysyl-tRNA synthetase can be MbPylRS. In some embodiments, said pyrrolysyl-tRNA synthetase can be MmPylRS. Specifically, the pyrrolysyl-tRNA synthetase may have an IPYE mutation (V31I/T56P/H62Y/A100E) compared to its wild type.

In the chimeric alanyl-tRNA synthetase, said tRNA binding domain (TD) may comprise the sequence as shown in any one of SEQ ID NOs: 1-15, 86-87, or a variant thereof. The chimeric alanyl-tRNA synthetase used in the present application can be chimeric with the catalytic domain (CD) of another aminoacyl-tRNA synthetase, thereby enabling said chimeric alanyl-tRNA synthetase to possess the function of catalyzing the aminoacylation of tRNAs other than alanine tRNA.

The chimeric alanyl-tRNA synthetase used in the present application can also be derived from alanyl-tRNA synthetase (AlaRS).

The alanyl-tRNA synthetase can be derived from eubacteria or archaea, or be a variant of an enzyme derived from eubacteria or archaea. The archaea can be one or more of Mj, Mm, Mb, Ma, G1, Lum1, 1R26, Int, Nitra, Deb, Mt, Methanococcus maripaludis, Methanopyrus kandleri, Halobacterium, Af, Pf, Ph, Pyrobaculum aerophilum, Pyrococcus abyssi, Ss, Sulfolobus tokodaii, Ap, Thermoplasma acidophilum, and Thermoplasma volcanium.

The eubacteria can be one of Escherichia coli, Thermus thermophilus, Bacillus subtilis, and Bacillus stearothermophilus.

The alanyl-tRNA synthetase can be derived from Escherichia coli.

The catalytic domain (CD) of said alanyl-tRNA synthetase comprises the sequence as shown in any one of SEQ ID NOs: 16-25, 88-111, or a variant thereof.

The tRNA binding domain (TD) of said chimeric alanyl-tRNA synthetase can be derived from a pyrrolysyl-tRNA synthetase, and the catalytic domain (CD) can be derived from an alanyl-tRNA synthetase.

The tRNA binding domain (TD) of said chimeric alanyl-tRNA synthetase can be derived from MbPylRS and/or MmPylRS, and the catalytic domain (CD) can be derived from an alanyl-tRNA synthetase of Escherichia coli. The N-terminus of the tRNA binding domain (TD) of MbPylRS and/or MmPylRS can be connected to the C-terminus of the catalytic domain (CD) of the Escherichia coli alanyl-tRNA synthetase. The C-terminus of the tRNA binding domain (TD) of MbPylRS and/or MmPylRS can be connected to the N-terminus of the catalytic domain (CD) of the Escherichia coli alanyl-tRNA synthetase.

The tRNA binding domain (TD) of said chimeric alanyl-tRNA synthetase can be derived from MbPylRS, and the catalytic domain (CD) can be derived from an alanyl-tRNA synthetase of Escherichia coli. The N-terminus of the tRNA binding domain (TD) of MbPylRS can be connected to the C-terminus of the catalytic domain (CD) of the Escherichia coli alanyl-tRNA synthetase. The C-terminus of the tRNA binding domain (TD) of MbPylRS can be connected to the N-terminus of the catalytic domain (CD) of the Escherichia coli alanyl-tRNA synthetase.

The tRNA binding domain (TD) of said chimeric alanyl-tRNA synthetase can be derived from MmPylRS, and the catalytic domain (CD) can be derived from an alanyl-tRNA synthetase of Escherichia coli. The N-terminus of the tRNA binding domain (TD) of MmPylRS can be connected to the C-terminus of the catalytic domain (CD) of the Escherichia coli alanyl-tRNA synthetase. The C-terminus of the tRNA binding domain (TD) of MmPylRS can be connected to the N-terminus of the catalytic domain (CD) of the Escherichia coli alanyl-tRNA synthetase.

There may be a connecting molecule between the tRNA binding domain (TD) and the catalytic domain (CD) of said chimeric alanyl-tRNA synthetase, and said connecting molecule may be a linker peptide. Said linker peptide may comprise the sequence as shown in any one of SEQ ID NOs: 50-55, or a variant thereof.

The chimeric alanyl-tRNA synthetase may comprise the sequence as shown in any one of SEQ ID NOs: 26-35, 56-75, or a variant thereof.

The chimeric alanyl-tRNA synthetase may comprise one or more efficiency-enhancing mutations. For example, the chimeric alanyl-tRNA synthetase may comprise an IPYE mutation (V31I/T56P/H62Y/A100E) to improve the enzymatic efficiency of the chimeric alanyl-tRNA synthetase. For example, chPylRS, MmPylRS, or MbPylRS may further comprise an IPYE mutation (V31I/T56P/H62Y/A100E) compared to their wild types.

In the above-mentioned method, the first portion of said recombinant tRNA can match the tRNA binding domain on the aminoacyl-tRNA synthetase.

The first portion of the recombinant tRNA can include, but is not limited to, one or more of the following parts: anticodon loop region, anticodon loop arm, anticodon, acceptor arm, D-loop, D-arm, TY′C arm, TY′C loop, or variable loop.

The first portion of the recombinant tRNA can further include a recognition element for the aminoacyl-tRNA synthetase, and said recognition element can consist of one or more nucleotides or one or more base pairs. The aminoacyl-tRNA synthetase can bind to the recombinant tRNA by recognizing the recognition element on the first portion via the tRNA binding domain.

The recombinant RNA can have one or more identical recognition elements, or multiple different recognition elements. The recognition elements of different recombinant tRNAs can be located at different parts of the tRNA. The recognition element can be located in the anticodon loop region, anticodon arm, anticodon, acceptor arm, D-loop, D-arm, TY′C arm, TY′C loop, and/or variable loop of the tRNA. For example, for an alanine tRNA, the G3.U70 or A3. U70 base pair on the acceptor arm is the recognition element for the alanyl-tRNA synthetase to aminoacylate the alanyl-tRNA. “G3· U70” means that for a tRNA, the 3rd position is G and the 70th position is U. “A3. U70” means that for a tRNA, the 3rd position is A and the 70th position is U.

The first portion of said recombinant tRNA can be derived from other tRNAs, such as pyrrolysyl-tRNA.

The first portion of said recombinant tRNA can match the tRNA binding domain on MbPylRS and/or MmPylRS. The first portion of said recombinant tRNA can be derived from MbPylT and/or MmPylT. The first portion of said recombinant tRNA can be derived from the anticodon loop region, anticodon loop arm, anticodon, acceptor arm, D-loop, D-arm, TY′C arm, T′C loop, and/or variable loop of MbPylT and/or MmPylT.

The second portion of said recombinant tRNA is the region on the recombinant tRNA capable of undergoing an aminoacylation reaction with an amino acid, said aminoacylation reaction being completed under the catalysis of the catalytic domain (CD) of the aminoacyl-tRNA synthetase.

The second portion of said recombinant tRNA can be the acceptor arm of the tRNA, and said acceptor arm can include the CCA-OH at the 3′ end of the recombinant tRNA molecule. For example, under the catalysis of the aminoacyl-tRNA synthetase, the carried amino acid molecule is linked to the hydroxyl group at the 3′ end of the recombinant tRNA molecule via an esterification reaction to form an aminoacyl-tRNA.

The second portion of said recombinant tRNA can be derived from other tRNAs, such as the alanyl-tRNA of Escherichia coli. The second portion of said recombinant tRNA matches the alanyl-tRNA synthetase of Escherichia coli. The second portion of said recombinant tRNA can be derived from the acceptor arm of the alanyl-tRNA of Escherichia coli.

In some embodiments, the second portion of said recombinant tRNA has an A-U mutation compared to the wild-type Escherichia coli alanyl-tRNA.

The recombinant tRNA may comprise the sequence as shown in any one of SEQ ID NOs: 36-49, or a variant thereof.

The present application also provides a method for producing a protein or polypeptide comprising one or more 2-aminoisobutyric acid and its analogs (Aib) in a host cell, said method comprising culturing the host cell with the following substances: a chimeric alanyl-tRNA synthetase (chAlaRS) or a nucleic acid encoding the chimeric alanyl-tRNA synthetase, and a recombinant tRNA or a nucleic acid encoding the recombinant tRNA, wherein said chimeric alanyl-tRNA synthetase comprises a tRNA binding domain (TD) and a catalytic domain (CD), wherein said tRNA binding domain (TD) is derived from a pyrrolysyl-tRNA synthetase, and the catalytic domain (CD) can be derived from an alanyl-tRNA synthetase; wherein said recombinant tRNA comprises a first portion and a second portion, wherein the first portion is derived from the anticodon loop region, anticodon loop arm, anticodon, acceptor arm, D-loop, D-arm, TY′C arm, TY′C loop, and/or variable loop of PylT, and the second portion is derived from the acceptor arm of an alanyl-tRNA.

The tRNA binding domain (TD) of said chimeric alanyl-tRNA synthetase can be derived from MbPylRS and/or MmPylRS, and the catalytic domain (CD) can be derived from an alanyl-tRNA synthetase of Escherichia coli. The N-terminus of the tRNA binding domain (TD) of MbPylRS and/or MmPylRS can be connected to the C-terminus of the catalytic domain (CD) of the Escherichia coli alanyl-tRNA synthetase. The C-terminus of the tRNA binding domain (TD) of MbPylRS and/or MmPylRS can be connected to the N-terminus of the catalytic domain (CD) of the Escherichia coli alanyl-tRNA synthetase.

The tRNA binding domain (TD) of said chimeric alanyl-tRNA synthetase can be derived from MbPylRS, and the catalytic domain (CD) can be derived from an alanyl-tRNA synthetase of Escherichia coli. The N-terminus of the tRNA binding domain (TD) of MbPylRS can be connected to the C-terminus of the catalytic domain (CD) of the Escherichia coli alanyl-tRNA synthetase. The C-terminus of the tRNA binding domain (TD) of MbPylRS can be connected to the N-terminus of the catalytic domain (CD) of the Escherichia coli alanyl-tRNA synthetase.

The tRNA binding domain (TD) of said chimeric alanyl-tRNA synthetase can be derived from MmPylRS, and the catalytic domain (CD) can be derived from an alanyl-tRNA synthetase of Escherichia coli. The N-terminus of the tRNA binding domain (TD) of MmPylRS can be connected to the C-terminus of the catalytic domain (CD) of the Escherichia coli alanyl-tRNA synthetase. The C-terminus of the tRNA binding domain (TD) of MmPylRS can be connected to the N-terminus of the catalytic domain (CD) of the Escherichia coli alanyl-tRNA synthetase.

There may be a connecting molecule between the tRNA binding domain (TD) and the catalytic domain (CD) of said chimeric alanyl-tRNA synthetase, and said connecting molecule may be a linker peptide. Said linker peptide may comprise the sequence as shown in any one of SEQ ID NOs: 50-55, or a variant thereof.

The chimeric alanyl-tRNA synthetase may comprise the sequence as shown in any one of SEQ ID NOs: 26-35, 56-75, or a variant thereof.

The recombinant tRNA may comprise the sequence as shown in any one of SEQ ID NOs: 36-49, or a variant thereof.

In said method, said AiB may have the following structure:

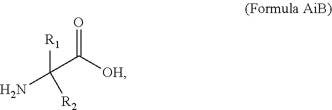

wherein R1 and/or R2 can be C₁₋₅ alkyl. R1 and/or R2 can be methyl.

In the above mentioned method, the protein or polypeptide can be a GLP-1 analog. In some embodiments, said polypeptide comprises the sequence as shown in any one of SEQ ID NOs: 76-85.

The present application also provides a method for producing a GLP-1 analog comprising one or more 2-aminoisobutyric acid in a host cell, said method comprising culturing the host cell with the following substances: a chimeric alanyl-tRNA synthetase (chAlaRS) or a nucleic acid encoding the chimeric alanyl-tRNA synthetase, and a recombinant tRNA or a nucleic acid encoding the recombinant tRNA; wherein said chimeric alanyl-tRNA synthetase comprises a tRNA binding domain (TD) and a catalytic domain (CD), wherein said tRNA binding domain (TD) is derived from MbPylRS and/or MmPylRS, and the catalytic domain (CD) is derived from an alanyl-tRNA synthetase of Escherichia coli; wherein said recombinant tRNA comprises a first portion and a second portion, wherein the first portion is derived from the anticodon loop region, anticodon loop arm, anticodon, acceptor arm, D-loop, D-arm, T′C arm, TY′C loop, and/or variable loop of MbPylT and/or MmPylT, and the second portion is derived from the acceptor arm of the Escherichia coli alanyl-tRNA.

In said method, the protein or polypeptide can be a GLP-1 analog. In some embodiments, said polypeptide comprises the sequence as shown in any one of SEQ ID NOs: 76-80, 112-117.

Aib, D-me-cys, and D-isoval are noncanonical amino acids, with structures as shown in FIG. 7.

The present application also provides the use of said noncanonical amino acid genetic encoding system in the preparation of polypeptides containing one or more Aib residues. Without wishing to be bound by any theory, the following examples are merely for illustrating the fusion proteins, preparation methods, and uses of the present application, and are not intended to limit the scope of the invention of the present application.

### EXAMPLES

The specific embodiments of the invention are illustrated below by specific examples. Those skilled in the art can easily understand other advantages and effects of the invention from the contents disclosed in this specification.

### Example 1: GFP Amber Suppression Assay

The pBK plasmid containing the orthogonal aminoacyl-tRNA synthetase and the pNEG plasmid containing GFP190TAG were co-transformed into E. coli DH10B cells. The cells were plated on plates containing corresponding antibiotics and cultured overnight at 37° C.

Three single colonies were picked from the transformed plates into LB medium containing corresponding antibiotics and cultured with shaking until the OD600 reached approximately 0.8. Corresponding non-natural amino acids were added, and L-arabinose was added to a final concentration of 0.2% to induce expression. A control without the addition of non-natural amino acids was set up. The culture was continued with shaking at 30° C. for 16 h.

After expression, 750 μL of cell culture was centrifuged, the medium was removed, and the cells were lysed with 150 μl of BugBuster protein extraction reagent (Millipore). After lysis, the mixture was centrifuged at 12,000 rpm for 1 min. 100 μL of the supernatant was transferred to a 96-well culture plate (COSTAR). The GFP signal of the supernatant was recorded using Bio Tek Synergy NEO2 and normalized to the OD600 value of the culture.

### Example 2: Construction of Chimeric Alanyl-tRNA Synthetase/tRNA

2.1 Construction of Chimeric Alanine tRNA Expression Vector

In the present invention, the chimeric tRNA and the reporter gene GFP190TAG-His6 were constructed on the same plasmid, pNEG, controlled by the lpp promoter and pBAD promoter, respectively. Simultaneously, a pNEG vector containing the chimeric tRNA, the reporter gene GFP190TAG-His6, and the resistance selection gene CAT112TAG was constructed, controlled by the lpp promoter, pBAD promoter, and Trp promoter, respectively.

The detailed sequences of the chimeric alanine tRNAs are shown in SEQ ID NOs: 36-49. Primers were designed to construct these tRNAs into the pNEG-GFP190TAG and pNEG-GFP190TAG-CAT112TAG vectors, respectively. The corresponding primers are shown in Table 1.

TABLE 1
Primers required for constructing chimeric alanine tRNAs

| | | SEQ |
|---|---|---|
| | | ID |
| Name | Sequence | NO |
| ChAla | TTCGCTAAGGATCTGCAGTGGTGGAGCTACCGG | 118 |
| T-1-F | | |
| ChAla | CTACATGATCATAGCTCCGAATTCAGCGTTACAAG | 119 |
| T-1-R | | |
| ChAla | CGCTAAGGATCTGCAGTGGTGGACTCGCCGGGAATCTAAC | 120 |
| T-2-F | CCGGC | |
| ChAla | CTTGTAACGCTGAATTCGGACTCGTGATCATGTAGATCGA | 121 |
| T-2-R | ACG | |
| ChAla | CTAAGGATCTGCAGTGGTAAGGACTCCGGGAATCTAACCC | 122 |
| T-3-F | GGCTG | |
| ChAla | GTCCGTTCGATCTACATGATCAAGCCCTTGAATTCAGCGTT | 123 |
| T-3-R | ACAAG | |
| ChAla | CTAAGGATCTGCAGTGGTGGACTCGCGGGGGGTCGAACCC | 124 |
| T-4-F | CGCTTAC | |
| ChAla | GACCCGTTTGCTCGCCGGAGCCGAGTCCGAATTCAGCGTT | 125 |
| T-4-R | ACAAG | |
| ChAla | CGCTAAGGATCTGCAGTGGTGGAAGAGCCGGGAATCGAA | 126 |
| T-5-F | CC | |
| ChAla | GTTCGGCCACCTTGGCCACTCTTCCGAATTCAGCGTTACAA | 127 |
| T-5-R | GTATT | |
| pNEG- | GAATTCAGCGTTACAAGTATTACACAAAGTTTTTTATGTTG | 128 |
| V-F | | |
| pNEG- | CTTAAAAAAAATCCTTAGCTTTCGCTAAGGATCTGCAGTG | 129 |
| V-R | G | |

### 2.2 Construction of Chimeric Alanyl-tRNA Synthetase

The chimeric alanyl-tRNA synthetase consists of two parts: one part is the tRNA binding domain of a pyrrolysyl-tRNA synthetase, and the other part is the catalytic domain of an alanyl-tRNA synthetase, connected by a linker peptide. The amino acid sequences of the tRNA binding domain of the pyrrolysyl-tRNA synthetase selected in the example are shown in SEQ ID NOs: 1-15, 86-87; the selected catalytic domain of the alanyl-tRNA synthetase is shown in SEQ ID NOs: 16-25, 88-111; and the preferred linker peptide sequences are shown in SEQ ID NOs: 50-55. Primers were designed to PCR amplify the three parts and ligate them into the pBK vector. The primers used are shown in Table 2.

TABLE 2
Primers required for constructing chimeric alanyl-tRNA synthetase

| | | SEQ |
|---|---|---|
| | | ID |
| Name | Sequence | NO |
| Pyl- | GGCGGAAGTGGTGGCGGAAGCATGGATAAGAAGCCGCTGGA | 130 |
| F1 | TGTTC | |
| Pyl- | GCGTTTGAAACTGCAGTTAAGAGGTCGGAACCGGAGAGTTC | 131 |
| R1 | GGGG | |
| Pyl- | GGCGGAAGTGGTGGCGGAAGCATGGATAAGAAGCCGCTGGA | 130 |
| F2 | TGTTC | |
| Pyl- | GCCGCCGCTTCCGCCACCCTCGCGTTCCTCAGCGTAGATCTGT | 132 |
| R2 | TG | |
| Pyl- | CGCTTTGAGGAATCCCATATGGTGGTCAAGTTCACCGACAGC | 133 |
| F3 | | |
| Pyl- | CGTGATCGCCAATCCTCGGAACGGTGGCGGAAGCGGCGGCA | 134 |
| R3 | TGAC | |
| Ala- | CGCTTTGAGGAATCCCATATGAGCAAGAGCACCGCTGA | 135 |
| F1 | | |
| Ala- | CGCTTCCGCCGCCGCTTCCGCCACCTGCCAGCTCTTCATCCAG | 136 |
| R1 | CAACGCCAG | |
| Ala- | GTGGCGGAAGCGGCGGCATGACTCTCGACGAGGAGTATCT | 137 |
| F2 | | |
| Ala- | GCTGGCAAAGAGGCACTCATAACTGCAGTTTCAAACGCT | 138 |
| R2 | | |
| pBK | CTGCAGTTTCAAACGCTAAATTGCCTGATGCGC | 139 |
| -V-F | | |
| pBK | GTTATACGTTGTTTACGCTTTGAGGAATCCCAT | 140 |
| -V-R | | |

### Example 3: Screening of Chimeric Alanyl-tRNA Synthetase Recognizing Aib

### 3.1 Construction and Screening of Chimeric Alanyl-tRNA Synthetase Mutant Library

Auto Dock Vina was used to dock the non-natural amino acid (taking diaminobutyric acid (Aib) as an example) into the substrate binding pocket of the chimeric alanyl-tRNA synthetase. Sites within 4 Å of Aib were selected for saturation mutagenesis, specifically selecting positions 147, 191, 200, 212, 214, 242, and 243. The designed primers are shown in Table 3.

TABLE 3
Construction of saturation mutagenesis library

| | | SEQ |
|---|---|---|
| | | ID |
| Name | Sequence | NO |
| 147-F | CATCTTACGCTTTTCGAGnnkATGGCTCATCATGCG | 141 |
| | TTCAACTATC | |
| 147-R | GAACGGGAAGGCATCTTACGCTTTTCGAG | 142 |
| 191-F | GTTTACAAGGAGGAGCCGnnkGCGGGTGGGGGTAAT | 143 |
| | GCTGGC | |
| 191-R | GGAAGACATTGTTTACAAGGAGGAGCCG | 144 |
| 214-F | CTTGAGGTTGCCACGCTGnnkTTCATGAACCTTGA | 145 |
| | GGAGCATC | |
| 214-R | CGGCGGTCTTGAGGTTGCCACGCTG | 146 |
| 242/243 | GGCTACGGTCTTGAAAGGTTCGTGTGGGCG | 147 |
| -F | | |
| 242/243 | CAGAAAAATGGACAACTACATCGTTnnkACTGGCTA | 148 |
| -R | CGGTCTTG | |
| 212/214 | GTCTTGAGGTTGCCNNKCTGnnkTTCATGAACCTT | 149 |
| -F | CGAGGAGATC | |
| 212/214 | GGCAACCTCAAGACCGCCGACTATGGCCTC | 150 |
| -R | | |
| pBK-V- | CTGCAGTTTCAAACGCTAAATTGCCTGATGCGC | 139 |
| F | | |
| pBK-V- | GTTATACGTTGTTTACGCTTTGAGGAATCCCAT | 140 |
| R | | |

Using the above primers, the corresponding fragments were amplified by PCR and ligated into the pBK vector using the Gibson assembly method. The assembled library plasmids were transformed into DH10B competent cells containing pNEG-chAlaT-GFP190TAG-his-CAT112TAG by electroporation. The transformed cells were recovered in 0.9 ml LB medium at 37° C. for 1 h, and then plated on LB agar plates containing 50 μg/ml kanamycin, 100 μg/ml ampicillin, 20 μg/ml chloramphenicol, 2 mM Aib, and 0.2% L-arabinose. After incubation at 37° C. for 24 h and subsequently at 22° C. for 72 h, fluorescent clones were picked from the plates using Azure BioC400 on the Cy2 channel and inoculated into 96-well culture plates. The cells were cultured at 37° C., 220 rpm for 10 hours, then 2 mM Aib was added to the medium, and expression was induced with 0.2% arabinose for 16 h. OD600 and GFP fluorescence (Fex=490/10 nm, Fem=510/10 nm) were recorded using Bio Tek Synergy NEO2. Cells with the highest GFP/OD600 ratio were picked and inoculated into LB medium containing 100 μg/ml ampicillin, and plasmids were extracted using a plasmid miniprep kit. The extracted DNA was digested with EcoRI restriction enzyme to remove the pNEG-chAlaT-GFP190TAG-his-CAT112TAG plasmid and transformed into E. coli DH10B competent cells. The pBK plasmids containing ChAlaRS variants were extracted and sequenced. Screening results are shown in FIG. 1. The selected enzymes are collectively referred to as AibRS (SEQ ID NO 56-75). For example, the screening results in (B) of FIG. 2 are the enzymes corresponding to SEQ ID NO 56.

3.2 LC-MS Analysis of Fidelity of Non-Natural Amino Acid Incorporation into Proteins

To verify the fidelity of the chimeric alanyl-tRNA synthetase mutant/tRNA in recognizing Aib, the GFP protein needed to be purified and determined by LC/MS. The constructed chimeric alanyl-tRNA synthetase plasmid and the plasmid carrying chimeric alanine tRNA and GFP-190TAG were co-transformed into DH10B competent cells. After recovery, the cells were plated on LB double-resistance plates containing 50 μg/ml kanamycin sulfate (kan) and 100 μg/ml ampicillin (Amp). Single colonies were picked into LB medium containing 50 μg/ml kan and 100 μg/ml Amp and cultured overnight. The culture was inoculated at a ratio of 1:100 into 100 ml of LB medium containing kan and Amp. When the OD600 reached 0.8, L-arabinose (working concentration 0.2%) and Aib were added, and GFP expression was induced at 22° C. and 220 rpm for 14 h. After induction, cells were collected by centrifugation at 4000 rpm at 22° C., resuspended in pre-cooled NTA-0 buffer (25 mM Tris, 250 mM NaCl, pH 8.0), and then disrupted by ultrasonication. The bacterial lysate was centrifuged at 10,000 rpm for 30 min at 4° C. to collect the supernatant. The obtained supernatant was loaded onto a nickel affinity chromatography chelating column equilibrated with NTA-0 buffer, then washed with 10 column volumes of NTA-0 buffer containing 50 mM imidazole, and finally the target protein was eluted with NTA-0 buffer containing 500 mM imidazole and subjected to SDS-PAGE and LC/MS analysis.

In LC-MS, the purified protein was analyzed on an LCQ Deca XP MAX mass spectrometer (Thermo Fisher Science) equipped with an electrospray ionization (ESI) source and an Agilent 1200 HPLC. Separation and desalting were performed on an Agilent 300 SB-C18 column (300× 2.1, 150 mm, 5 μm) with mobile phase A being an aqueous solution containing 0.1% formic acid and mobile phase B being acetonitrile containing 0.1% formic acid, with the flow rate set at 0.2 ml/min. Data were analyzed using XCalbur-Quar browser software. Mass spectral deconvolution was performed using the Core Bayesian deconvolution algorithm in UniDec software (version 2.6.8, University of Oxford). The theoretical molecular weight of the protein was predicted using the ExPASy Compute pI/Mw tool (https://web.expasy.org/compute_pi/). The results showed that the screened AibRS could recognize Aib (SEQ ID NO: 56-75), and the fidelity was greater than 95%.

### Example 4: Recombinant Expression of Semaglutide Backbone

### 4.1 Intracellular Soluble Expression and Purification of Semaglutide Backbone

### (1) Construction of Semaglutide Backbone Soluble Expression Vector

To increase the expression level of Semaglutide in E. coli and facilitate subsequent purification, a His tag, a SUMO tag, and the Semaglutide backbone were fused in tandem to express a fusion protein (amino acid sequence), although the expression strategy is not limited to this method. In the expression of the Semaglutide backbone, the leader peptide of the fusion expression polypeptide can be TrxA, DsbA, DsbC, Sumo, GST, MBP, Intein.

Taking the fusion of the Sumo tag as an example, the amino acid sequence of the fusion protein is shown in SEQ ID NO: 81. Based on the amino acid sequence of Semaglutide, an expression sequence was designed wherein the codon for Ala at position 8 was mutated to TAG. Primers were designed to construct the fusion protein into the corresponding vector to generate plasmid pNEG-Sumo-GLP1-8TAG-2*chAlaT (SEQ ID NO: 81).

### (2) Soluble Expression and Purification of Semaglutide Backbone Fusion Protein

The vector pNEG-Sumo-GLP1-8TAG-2*chAlaT and the plasmid carrying AibRS (SEQ ID NO 60) were co-transformed into E. coli competent cells. After screening on plates to obtain single clones, single colonies were picked into LB medium and cultured at 37° C., 220 rpm until OD600-0.8. Aib was added to a final concentration of 2 mM, and IPTG and arabinose were added to induce expression at 22° C. for 15 h.

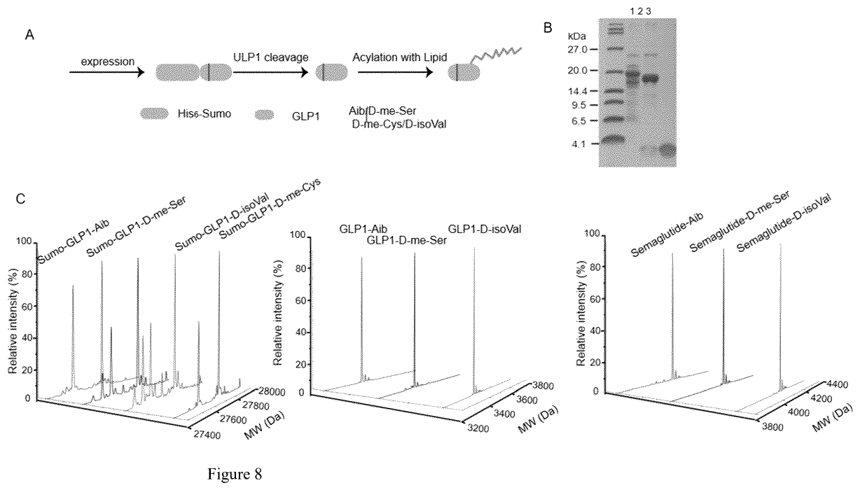

The bacterial liquid induced for 15 h was centrifuged at 4000 rpm for 15 min to collect the cells. The cells were resuspended using resuspension buffer, ultrasonicated on ice at 150 W for 15 min, and then centrifuged at 10,000 rpm for 30 min at 4° C. to collect the supernatant. Affinity chromatography was used to purify the target fusion protein. The supernatant was loaded onto an equilibrated nickel affinity chromatography chelating column. Wash buffer was used to wash away impurity proteins on the nickel column, and finally, eluent buffer was used to elute the target polypeptide. Resuspension buffer composition: Tris (20 mM), NaCl (250 mM), pH adjusted to 7.5 with concentrated hydrochloric acid. Wash buffer: imidazole added to resuspension buffer to a concentration of 50 mM. Eluent buffer: imidazole added to resuspension buffer to a concentration of 500 mM. The purified sample was concentrated using a 10K ultrafiltration tube, buffer exchanged into pure water, and subjected to SDS-PAGE and LC/MS analysis. The results are shown in FIG. 8.
(3) Purification of Semaglutide Backbone The purified fusion protein was digested with SUMO protease overnight at 30° C. Since the SUMO protease also contains a HIS tag, the digested sample was passed through a nickel affinity chromatography chelating column to remove the SUMO protease and the SUMO tag. The flow-through was collected and concentrated/buffer exchanged using a 3K ultrafiltration tube to obtain the Semaglutide backbone. Further analysis was performed using Tricine SDS-PAGE and LC/MS.

TABLE 4

Preparation of SUMO Digestion Stock Solution
10× SUMO Protease Buffer + Salt
500 mM Tris-HCl, pH 8.0
10 mM DTT
1.5M NaCl

### 4.2 Secretory Expression of Semaglutide Backbone

### (1) Construction of Semaglutide Secretory Expression Vector

To achieve secretory expression, Semaglutide needs to be fused with a secretion signal peptide to mediate the secretion of Semaglutide. The secretion signal peptide can be selected from pelb (MKYLLPTAAAGLLLLAAQPAMA, SEQ ID NO 151), Mae (MKIKTGARILALSALTTMMFSASALA, SEQ ID NO 152), DsbA (MKKIWLALAGLVLAFSASA, SEQ ID NO 153), etc. Taking Mae as an example, primers were designed to fuse it using overlap extension PCR to obtain the coding sequence of the MalE signal peptide-Semaglutide fusion protein (SEQ ID NO: 82), which was ligated into the pET vector to construct pET-Mae-GLP1-8TAG.

### (2) Secretory Expression of Semaglutide Backbone

pET-Mae-GLP1-8TAG and the plasmid carrying AibRS/chAlaT were co-transformed into E. coli competent cells. Co-transformed strains were picked and inoculated into pH 7.2 FA medium (0.1% glucose (w/v), 1.5% acid hydrolyzed casein (w/v), 0.1% yeast extract (w/v), 0.04% MgSO4 (w/v), 0.5% (NH4)2SO4 (w/v), 0.4% KCl (w/v), 0.12 mol/L triethanolamine). After culturing at 37° C. until the OD reached 0.8-1.0, IPTG was added to a final concentration of 1 mM to induce the expression of the full-length Semaglutide peptide and the orthogonal synthetase, and the non-natural amino acid Aib was simultaneously provided at a final concentration of 1.5 mg/ml. After 18 hours, the culture supernatant was taken and the secretion results were characterized by SDS-PAGE using a Tris-Tricine buffer system and mass spectrometry.

### 4.3 Tandem Expression of Semaglutide Backbone

This example provides a coding sequence for the tandem expression of full-length Semaglutide peptides. Specifically, the coding sequence is based on the amino acid sequence of Semaglutide, designing a tandem repeat sequence of (Semaglutide-cleavage site)-(Semaglutide-cleavage site)-(Semaglutide-cleavage site) using (Semaglutide-cleavage site) as a repeating unit, and adding a pro-translation sequence containing a cleavage site at the N-terminus of the repeat sequence. The nucleic acid coding sequence corresponding to the amino acid sequence was designed according to the codon preference and usage frequency of E. coli, taking into account mRNA secondary structure and uniform GC content distribution. The codon for alanine at position 8 of Semaglutide in each tandem repeat unit was mutated to TAG. After design, an Ncol cleavage site was added to the 5′ end of the coding sequence, and a NotI cleavage site was added to the 3′ end. The designed double, triple, and quadruple tandem repeat coding sequences for full-length Semaglutide peptides are shown in SEQ ID NOs: 83-85.

The above fragments were synthesized and ligated into the pRSFDuet vector by restriction digestion, named pRSFDuet-2xSemaglutide, pRSFDuet-3xSemaglutide, and pRSFDuet-4xSemaglutide. The obtained expression vectors pRSFDuet-2xSemaglutide, pRSFDuet-3xSemaglutide, and pRSFDuet-4xSemaglutide were co-transformed with the AibRS/chAlaT plasmid into E. coli competent cells, and single clones were picked from plates. The single clones were inoculated into LB medium, cultured at 37° C. until the OD reached 0.8-1.0, then cooled to 30° C. IPTG was added to a final concentration of 1 mM to induce the expression of the full-length Semaglutide peptide and the orthogonal synthetase, and the non-natural amino acid Aib was simultaneously provided at a final concentration of 1.5 mg/ml. Cells were harvested after 18 hours. After ultrasonic lysis, the mixture was centrifuged again at 12,000 g, the supernatant was discarded, and the precipitate was dissolved in 8 M urea at 4° C. for 5 h. The supernatant was collected after centrifugation, which contained the tandem Semaglutide. A cleavage buffer exchange operation was performed on the tandem full-length Semaglutide peptide dissolved in 8 M urea using an ultrafiltration tube with a molecular weight cutoff of 3 kDa. The cleavage buffer was a Tris-HCl buffer at pH 8.0-9.0. The buffer exchange operation was repeated at least five times to thoroughly remove the urea from the previous step to avoid interference with subsequent enzymatic digestion. After buffer exchange, the tandem full-length Semaglutide peptide in the cleavage buffer was quantified using the BCA method. Recombinant protease Kex2 and recombinant protease CPB were added at a mass ratio of tandem Semaglutide:protease of 1000:1, and digested with shaking at 25-35° C. overnight. The digestion reaction product was filtered and desalted, then separated and purified on an Agilent 300 SB-C18 column (300× 2.1, 150 mm, 5 μm). The mobile phase A was an aqueous solution containing 0.1% formic acid, and mobile phase B was acetonitrile containing 0.1% formic acid. The flow rate was set to 0.2 ml/min. The initial gradient was set to 90% phase A and 10% phase B, and the final gradient was set to 10% phase A and 90% phase B, with a gradient elution time of 30 min. The peaks of each fraction were collected and analyzed using an LCQ Deca XP MAX mass spectrometer (Thermo Fisher Science). After confirming the target fractions, they were retained. The purity of the obtained full-length Semaglutide peptide was not less than 99%.

### Example 5: Recombinant Expression of Tirzepatide Backbone

Similar to the sequence of Semaglutide fused and expressed in Example 4.1, a partial sequence of Semaglutide was replaced with the sequence of the Tirzepatide backbone to adopt the same strategy for fusion expression of Tirzepatide (amino acid sequence). Similarly, the fusion expression scheme is not limited to this; leader peptides such as TrxA, DsbA, DsbC, Sumo, GST, and Intein can still be used for fusion expression of Tirzepatide.

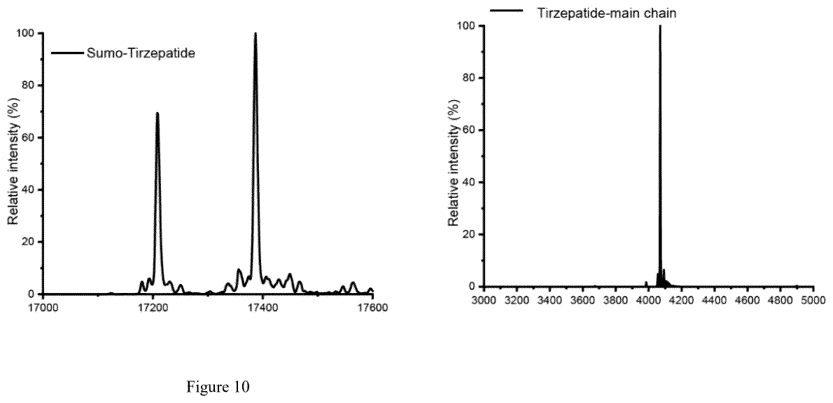

The corresponding nucleic acid sequence was designed according to the amino acid sequence of Tirzepatide. The nucleic acid sequences for the non-natural amino acids at positions 2 and 13 were set to TAG to achieve Aib insertion. After codon optimization for E. coli, it was constructed into the pNEG vector (base sequence) and named pNEG-Sumo-Tirzepatide-3/13TAG-2*chAlaT, but is not limited to this vector. For the expression and purification of the Tirzepatide backbone, see Example 4.2. Mass spectrometry results are shown in FIG. 10.

### Example 6: Genetic Encoding of Aib Analogs

The pBK-AibRS obtained in Example 3 and pNEG-GFP190TAG-chAlaT were co-transformed into DH10B competent cells. The transformed cells were cultured with shaking in LB medium at 37° C. for 1 h, then plated on LB agar plates containing 50 μg/ml kanamycin (kan) and 100 μg/ml ampicillin (AMP), and cultured at 37° C. for 12 h. Three points were picked from each plate into LB medium, cultured with shaking at 37° C. until OD600=0.8. Arabinose was added to a final concentration of 0.2%, and different non-natural amino acids were added. The culture was induced at 22° C. for 14 h to express the protein. After expression, 1 ml of cell culture was centrifuged, the medium was removed, and the cells were lysed with 150 μl of BugBuster protein extraction reagent (Millipore). After lysis, the mixture was centrifuged at 12,000 rpm for 1 min. 100 μl of the supernatant was transferred to a 96-well culture plate (COSTAR). The GFP signal of the supernatant was recorded using Bio Tek Synergy NEO2 and normalized. Proteins containing non-natural amino acids were expressed according to the method of Example 3.2, and the fidelity of insertion was verified by LC-MS. The results are shown in FIG. 7.

### Example 7: Incorporation of Aib and Analogs in Mammalian Cell Systems

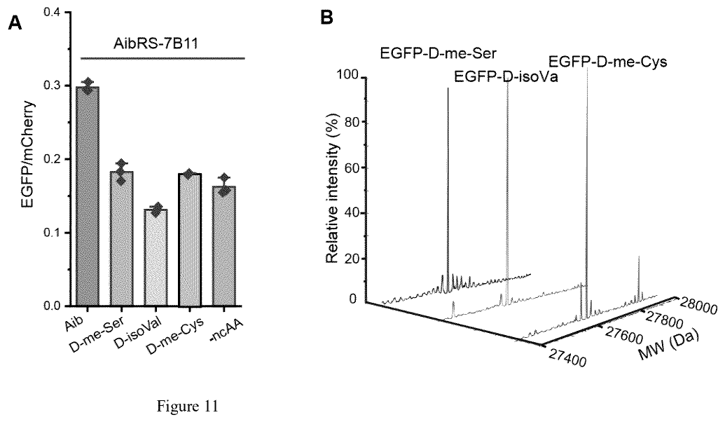

Primers were designed to construct AibRS (SEQ ID NO 60) and chAlaT into the pCMV vector, named pCMV-AibRS-chAlaT. This plasmid and the fluorescent reporter plasmid pEGFP-mCherry-T2A-EGFP-190TAG were co-transfected into HEK293T cells. 6 h after cell transfection, the medium was replaced with fresh medium, and the corresponding non-natural amino acid was added to a final concentration of 1 mM. After 48 h, the medium was aspirated, and 1×PBS was added to wash away residual medium. The PBS solution was aspirated, trypsin was added to digest the cells, and 1 mL of DMEM medium was added to resuspend the cells. The cells were transferred to a 1.5 mL centrifuge tube. The forward scatter and side scatter gates of the flow cytometer were set using HEK 293T cells. The parameters and gate for the PE channel were set using cells expressing mCherry, and the parameters and gate for the FITC channel were set using cells expressing EGFP. The experimental group cells were measured, with each sample set to collect 50,000 cells. To further verify the fidelity of non-natural amino acid insertion in mammalian cells, one 10 cm culture plate of cells was expressed for each, purified using nickel column affinity, and identified by LC-MS. The results are shown in FIG. 11.

### Example 8: Recombinant Expression of Other GLP-1 Analog and Fusion Protein Backbones

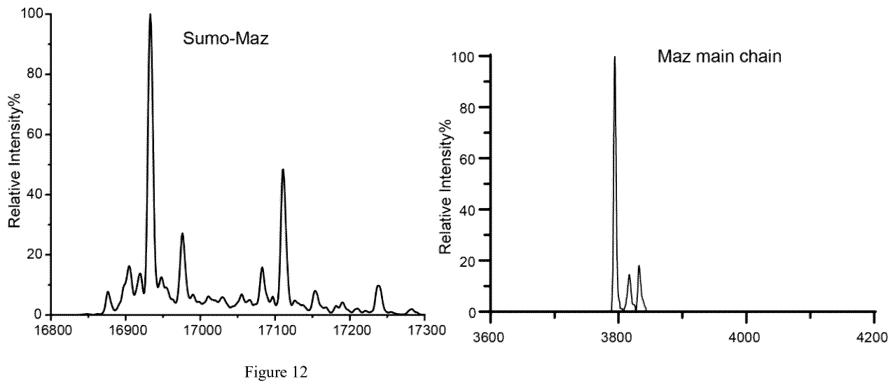

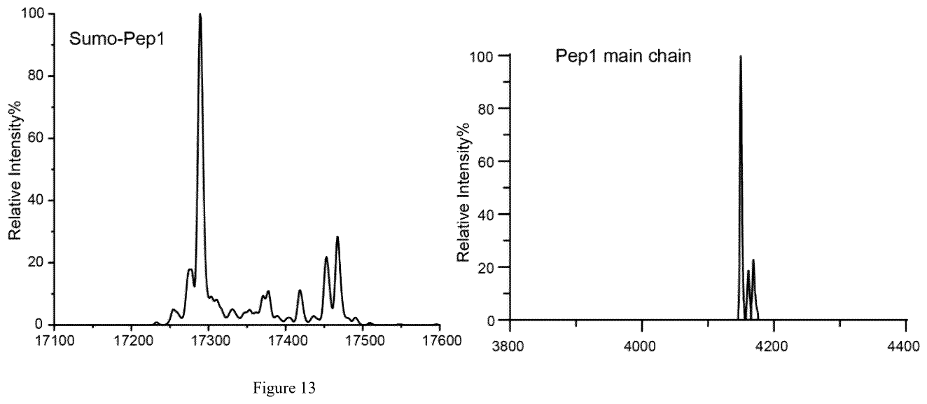

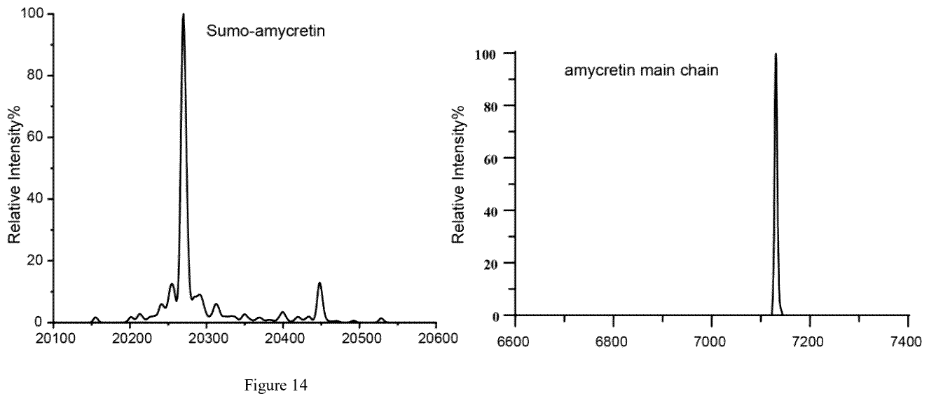

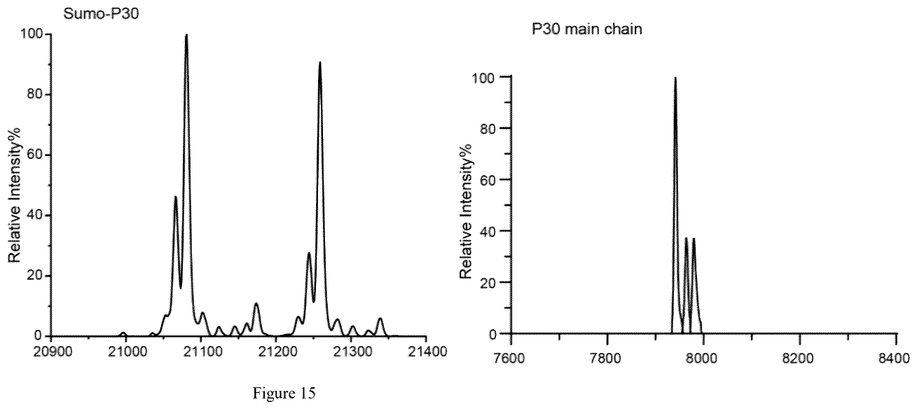

Similar to the biological expression methods for the Semaglutide and Tirzepatide backbones in Examples 4 and 5, this method is applicable to other polypeptides or fusion proteins containing Aib, including GLP-1/GCCG dual agonist Maz (SEQ ID NO: 112), GLP-1/GIP-1/GCCG tri-agonist (Pep1, 2, 3, SEQ ID NO: 113-115), GLP-1/Amylin dual agonist (amycretin, SEQ ID NO: 116), and GLP-1/GIP-1/Amylin tri-agonist (P30, SEQ ID NO: 117). Using the methods of Examples 4 and 5, this example successfully expressed and purified fusion proteins of these polypeptides and obtained the purified polypeptides. Mass spectrometry results are shown in FIGS. 12-15. In FIG. 12, AibRS is SEQ ID NO 61; in FIG. 13, AibRS is SEQ ID NO 64; and in FIGS. 14 and 15, AibRS is SEQ ID NO 60.

Part of the sequences of the present disclosure are shown as follow. X1, X2 and X3 represent any nucleotides. X1X2X3 represents an anticodon.

TABLE 5

| SEQ | | |
|---|---|---|
| ID | | |
| NO | LABEL | SEQ |
| 1 | 1Mb149 | MDKKPLDVLISATGLWMSRTGTLHKIKHHEVSRSKIYIEMACGDH |
| | | LVVNNSRSCRTARAFRHHKYRKTCKRCRVSDEDINNFLTRSTESK |
| | | NSVKVRVVSAPKVKKAMPKSVSRAPKPLENSVSAKASTNTSRSVP |
| | | SPAKSTPNSSVPAS |
| 2 | 2Mml49 | MDKKPLNTLISATGLWMSRTGTIHKIKHHEVSRSKIYIEMACGDH |
| | | LVVNNSRSSRTARALRHHKYRKTCKRCRVSDEDLNKFLTKANEDQ |
| | | TSVKVKVVSAPTRTKKAMPKSVARAPKPLENTEAAQAQPSGSKFS |
| | | PAIPVSTQESVSVP |
| 3 | 3ch149 | MDKKPLDVLISATGLWMSRTGTLHKIKHYEISRSKIYIEMACGDH |
| | | LVVNNSRSCRPARAFRYHKYRKTCKRCRVSDEDINNFLTRSTEGK |
| | | TSVKVKVVSEPKVKKAMPKSVSRADKELENPVSAKASTDTSRSVP |
| | | SPAKSTPNSPVPTS |
| 4 | 4G1 | MVVKFTDSQIQHLMEYGDNDWSEAEFEDAAARDKEFSSQFSKLKS |
| | | ANDKGLKDVIANPRN |
| 5 | 5Ma | MTVKYTDAQIQRLREYGNGTYEQKVFEDLASRDAAFSKEMSVAST |
| | | DNEKKIKGMIANPSR |
| 6 | 6lum | MDTRLTPAQAQRIREMGGTVDPSLAFSSEAERESAFQRISADLQG |
| | | ANLAKIRRCAEAPER |
| 7 | 7deb | MNSSWTEVQRHRLKELNGAEKDLETAFGDDLQRNRAFQKLEKQLV |
| | | YQERKRLDRLLDTRF |
| 8 | 8nitra | MASKIRFTRGQIHRLIELGAEPTELERDFETEAERDKEFNKIAEN |
| | | LARKNLKNIKDFLEQ |
| 9 | 91r26 | MAEHFTDAQIQRLREYGNGTYKDMEFADVSAREKAFTKLMSDASR |
| | | DNESALKGMIAHPAR |
| 10 | 10mbur | MEKQLLDVLVELNGVWLSRSGLLHGIRNFEITTKHIHIETDCGAR |
| | | FTVRNSRSSRSARSLRHNKYRKPCKRCRPADEQIDRFVKKTFKEK |
| | | RQTVSVFSSPKKHVPKKPKVAVIKSFSISTPSPKEASVSNSIPTP |
| | | SISVVKDEVKVPE |
| 11 | 11mm-ipye | MDKKPLNTLISATGLWMSRTGTIHKIKHHEVSRSKIYIEMACGDH |
| | | LVVNNSRSSRTARALRHHKYRKTCKRCRVSDEDLNKFLTKANEDQ |
| | | TSVKVKVVSAPTRTKKAMPKSVARAPKPLENTEAAQAQPSGSKFS |
| | | PAIPVSTQESVSVPASVSTSISSISTGATASALVKGNTNPITSMS |
| | | APVQASAPALTKSQTDRLEVLLNPKDEISLNSGKPFRELESELLS |
| | | RRKKDLQQIYAEERE |
| 12 | 12mb-ipye | MDKKPLDVLISATGLWMSRTGTLHKIKHYEISRSKIYIEMACGDH |
| | | LVVNNSRSCRPARAFRYHKYRKTCKRCRVSDEDINNFLTRSTEGK |
| | | TSVKVKVVSEPKVKKAMPKSVSRAPKPLENPVSAKASTDTSRSVP |
| | | SPAKSTPNSPVPTSAPAPSLTRSQLDRVEALLSPEDKISLNMAKP |
| | | FRELEPELVTRRKNDFQRLYTNDRED |
| 13 | 13ch-IPYE | MDKKPLDVLISATGLWMSRTGTLHKIKHYEISRSKIYIEMACGDH |
| | | LVVNNSRSCRPARAFRYHKYRKTCKRCRVSDEDINNFLTRSTEGK |
| | | TSVKVKVVSEPKVKKAMPKSVSRAPKPLENPVSAKASTDTSRSVP |
| | | SPAKSTPNSPVPTSASAPALTKSQTDRLEVLLNPKDEISLNSGKP |
| | | FRELESELLSRRKKDLQQIYAEERE |
| 14 | 14mm-comml | MNKKPLNTLISATGLWMSRTGTIHKIKHHEISRSKIYIEMACGDH |
| | | LVVNNSRSSRPARALKYHKYRKTCKRCRVSDEDLNKFLTKANEDQ |
| | | TSVKVKVVSAPTRTKKAMPKSVARAPKPLENSEAAQAQPSGSKFS |
| | | PAIPVSTQESVSVPASAPALTKRQTDRLEVLLNPKDEISLNSGKP |
| | | FRELESELLSRRKKDLQQIYAEERE |
| 15 | 15mm-comm2 | MNKKPLYTLISATGLWMSRTGTIHKIKHHEISRSKIYIEMACGDH |
| | | LVVNNSRSSRPARALKYLKYRNTCKRCRWSDEDLNKFLTKANEDQ |
| | | TSVKVKVVSAPTRTKKAMPKSVARAPKPLENSEAAQAQPSGSKFS |
| | | PAIPVSTQESVSVPASAPALTKSQTDRLEVLLNPKDEISLNSGKP |
| | | FRELESELLSRRKKDLQQIYAEERE |
| 16 | 16EcAlaRS-C | MSKSTAEIRQAFLDFFHSKGHQVVASSSLVPHNDPTLLFTNAGMN |
| | | QFKDVFLGLDKRNYSRATTSQRCVRAGGKHNDLENVGYTARHHTF |
| | | FEMLGNFSFGDYFKHDAIQFAWELLTSEKWFALPKERLWVTVYES |
| | | DDEAYEIWEKEVGIPRERIIRIGDNKGAPYASDNFWQMGDTGPCG |
| | | PCTEIFYDHGDHIWGGPPGSPEEDGDRYIEIWNIVFMQFNRQADG |
| | | TMEPLPKPSVDTGMGLERIAAVLQHVNSNYDIDLFRTLIQAVAKV |
| | | TGATDLSNKSLRVIADHIRSCAFLIADGVMPSNENRGYVLRRIIR |
| | | RAVRHGNMLGAKETFFYKLVGPLIDVMGSAGEDLKRQQAQVEQVL |
| | D | KTEEEQFARTLERGLALLDEELA |
| 17 | 17AfAlaRS-CD | MTLDEEYLDITFLTENGFVRKRCPKCGKHFWTADPEREICGDPPC |
| | | ESYSFIGNPVFKKPFELDEMREYYLNFFERRGHGRIERYPVVARW |
| | | RTDIYLTIASIADFQPFVTSGVAPPPANPLTISQPCIRLDDLDSV |
| | | GRTGRHLTLFEMMAHHAFNYPGKEIYWKNETVAYCTELLNELGVK |
| | | KEDIVYKEEPWAGGGNAGPCLEAIVGGLEVATLVFMNLEEHPEGD |
| | | IEIKGARYRKMDNYIVDTGYGLERFVWASKGTPTVYDAIFPEVVD |
| | | TIIDNSNVSFNREDERVRRIVAESSKLAGIMGELRGERLNQLRKS |
| | | VADTVGVSVEELEGIVVPLEKVYSLADHTRCILFMLGDGLVPSNA |
| | | GAGYLARLMIRRSLRLAEELELGLDLYDLVEMHKKILGFEFDVPL |
| | | STVQEILELEKERYRTTVSKGTRLVERLVERKKKLEKDDLIELYD |
| | | SHGIPVELAVGIAAEKGAEVEMPKDIYAELAKRHS |
| 18 | 18PhAlaRS-CD | MEFIMKTRMFEEEGWIRKKCKVCGKPFWTLDPDRETCGDPPCDEY |
| | | QFIGKPGIPRKYTLDEMREKFLRFFEKHEIYPHGRVKRYPVLPRW |
| | | RDDVLLVGASIMDFQPWVISGEADPPANPLVISQPSIRFTDIDNV |
| | | GITGRHFTIFEMMAHHAFNYPGKPIYWMDETVELAFEFFTKELKM |
| | | KPEDITFKENPWAGGGNAGPAFEVLYRGLEVATLVFMQYKKAPEN |
| | | APQDQVVVIKGEKYIPMETKVVDTGYGLERLVWMSQGTPTAYDAV |
| | | LGYVVEPLKKMAGIEKIDEKILMENSRLAGMFDIEDLGDLRYLRE |
| | | QVAKRVGITVEELEKAIRPYELIYAIADHTKALTFMLADGVVPSN |
| | | VKAGYLARLLIRKSIRHLRELGLEVPLSEIVALHIKELHKTFPEF |
| | | KEMEDIILEMIELEEKKYAETLRRGSDLVRREIAKLKKKGIKEIP |
| | | VEKLVTFYESHGLTPEIVKEIAEKEGVKVNIPDNFYSMVAK |
| 19 | 19HmAlaRS-CD | AWRGLSHRPLSSEPPAAKASAVRAAFLNFFRDRHGHRLVPSASVR |
| | | PRGDPSLLFVNAGMNQFKPIFLGTVDPRSEMAGFRRVANSQKCVR |
| | | AGGHHNDLEDVGRDLSHHTFFEMLGNWAFGGEYFKEEACNMAWEL |
| | | LTQVYGIPEERLWISYFDGDPKAGLDPDLETRDIWLSLGVPASRV |
| | | LSFGPQENFWEMGDTGPCGPCTEIHYDLAGGVGAPQLVELWNLVF |
| | | MQHNREADGSLQPLPQRHVDTGMGLERLVAVLQGKHSTYDTDLFS |
| | | PLLNAIQQGCRAPPYLGRVGVADEGRTDTAYRVVADHIRTLSVCI |
| | | SDGIFPGMSGPPLVLRRILRRAVRFSMEILKAPPGFLGSLVPVVV |
| | | ETLGDAYPELQRNSAQIANLVSEDEAAFLASLERGRRIIDRTLRT |
| | | LGPSDMFPAEVAWSLSLCGDLGLPLDMVELMLEEKGVQLDSAGLE |
| | | RLAQEEAQHRARQA |
| 20 | 20HsAlaRS-CD | MDSTLTASEIRQRFIDFFKRNEHTYVHSSATIPLDDPTLLFANAG |
| | | MNQFKPIFLNTIDPSHPMAKLSRAANTQKCIRAGGKHNDLDDVGK |
| | | DVYHHTFFEMLGSWSFGDYFKELACKMALELLTQEFGIPIERLYV |
| | | TYFGGDEAAGLEADLECKQIWQNLGLDDTKILPGNMKDNFWEMGD |
| | | TGPCGPCSEIHYDRIGGRDAAHLVNQDDPNVLEIWNLVFIQYNRE |
| | | ADGILKPLPKKSIDTGMGLERLVSVLQNKMSNYDTDLFVPYFEAI |
| | | QKGTGARPYTGKVGAEDADGIDMAYRVLADHARTITVALADGGRP |
| | | DNTGRGYVLRRILRRAVRYAHEKLNASRGFFATLVDVVVQSLGDA |
| | | FPELKKDPDMVKDIINEEEVQFLKTLSRGRRILDRKIQSLGDSKT |
| | | IPGDTAWLLYDTYGFPVDLTGLIAEEKGLVVDM |
| 21 | 21PfAlaRS-CD | MEFVMKTRMFEEEGWIRKTCKVCGKPFWTLDPDRETCGDPPCDEY |
| | | QFIGKPGIPKKYTLDEMREKFLSFFEKHEVYPHGRVKRYPVLPRW |
| | | RDDVLLVGASIMDFQPWVISGEADPPANPLTISQPSIRFTDIDNV |
| | | GITGRHFTIFEMMAHHAFNYPGKHIYWIDETVELAFEFFTKELKM |
| | | KPEDITFKENPWAGGGNAGPAFEVLYKGLEVATLVFMQYKKAPAN |
| | | ADPSQVVIIKGEKYVPMETKVVDTGYGLERLVWMSQGTPTAYDAV |
| | | LGYVIEPLKRMAGVEKIDERILMENSRLAGMFDIEDMGDLRYLRE |
| | | QVAKRVGISVEELERLIRPYELIYAIADHTKALTFMLADGVVPSN |
| | | VKAGYLARLLIRKSIRHLRELGLEVPLAEIVAMHIKELSPTFPEF |
| | | KEMEDIILEMIELEEKKYAETLRRGSDLVKREIAKLKKKGANEIP |
| | | LEKLITFYESHGLTPEIVKEIAEKEGVKVHIPDNFYSLVAK |
| 22 | 22MpAlaRS-CD | MLEDEYQLDFFSENGFVRKQCSKCGKFYWTRDLDRNTCGDAPCDP |
| | | YSFIGNPVFNKKFDLAEMREYYLKFFEDQGHTRLERYPVVARWRD |
| | | DIYLTIASIADFQPFVTSGQVRPPANPLTISQPCIRLSDLDAVGR |
| | | SGRHLTTFEMMAHHAFNKKGEEIYWKDRTMELCDELFNSLGVDPL |
| | | AVTYKEEPWAGGGNAGPCVEALIGGLEVATLVFMDLEQSKDGTIP |
| | | IKGENYRKMDNYIVDTGYGLERFVWASKGSPTIYDAVFPTIVNEL |
| | | MQLAGLEHQIDDPEYANILSQSARLAGLMDVSEKANLLQLRKQVA |
| | | SSIGTSVEKLSSIMEPVENIYAITDHTRCLTFMLADGIIPSNVKA |
| | | GYLARLVLRRTLRMMKDMDINTSLSDIVRMHIKNLPEYPEFSEKF |
| | | DVIEDILAHEEMKFDETLERGRRMIQKSAKHYKSTGEKIPLPTLI |
| | | EMYDSHGIPPEISKEAASEMGVEVDLPDNFYSLVAEGHS |
| 23 | 23TtA1aRS-CD | MLSTKLFETAGFHRKQCPMCKSYFWTLRQDQVYCGDQPCVPYGFI |
| | | GNSPVRVPVESIRDLRERFLSFFEKRGHARIKRYPVVARWREDVF |
| | | LVGASIYDFQPWVTSGAVPPPANPLTISQPSIRLTDVDKVGRSGR |
| | | HLTGFEMMAHHAFNYPDKHIYWISETTEYAYEFFTKELGIPEDEI |
| | | TFKESVWEGGGNAGESFEVLVRGLEVATLVFMHYEVKDGKYVELP |
| | | LKIVDTGYGLERIYWMAKGSPTVYDAVFGPFLSAARKKLGVPEPP |
| | | AHILGKASVYFGQMDPEVIGLAKAYDLVAEKIGIDPREFREIMRP |
| | | QEALYVLADHSRTVSWMIADGVIPSNTGAGYLARLLIRRALKSLW |
| | | IAGIETSLVDLFDLHLGFLRDDYPEVWQSRDIILELIDLEEKKYK |
| | | DVIKSAPAAVRRALEKAGGKSLKAEQLVELYDSYGIPPEVAQEVA |
| | | RKLGAEAEVPDDFYSRLAARHQ |
| 24 | 24TsAlaRS-CD | MSMDMTTRMFKEEGWIRKQCPKCGKFFWTLDPDRETCGDPPCDEY |
| | | SFIGKPGIPKKYTLEEMREAFLRFFEKHDHGRVKRFPVLPRWRDD |
| | | VLLVGASIMDFQPWVISGEADPPANPLTISQPSIRFTDIDNVGIT |
| | | GRHFTIFEMMAHHAFNYPGKPIYWMDETVELAFEFFTKELGMKAE |
| | | DITFKENPWAGGGNAGPAFEVLYRGLEVATLVFMQYKKAPENADP |
| | | SQVVEIKGDYYVPMETKVVDTGYGLERLVWMSHGTPTAYDAVLGY |
| | | VVEPLKRMAGIERIDERILMENSRLAGMFDIEDMGDLRYLRGQVA |
| | | KRVGISVEELEKAVRPYELIYAIADHTKALTFMLADGVIPSNVKA |
| | | GYLARLLIRKSIRHLRELGLEVPLAEIVAMHIKELSPTFPEFKEM |
| | | EDVILDIINVEEKRYQETLKRGSDLVKREIARLKKAGKNEMPLEK |
| | | LILFYESHGLTPEIVREVAEKEGVKVEIPDNFYTLVAK |
| 25 | 25PaAlaRS-CD | MSQPFRSADIRQAFIDFFISKQHTPVASSSLIPHNDPTLLFTNAG |
| | | MNQFKETFLGMEPRDYTRAVTSQKCVRAGGKHNDLDNVGYTARHH |
| | | TFFEMLGNFSFGDYFKQAGIGYIWEFLTSDEWLAIDKNRLYVTIY |
| | | ETDDEAFDIWHKDIGIPSERIIRIGDNKGAPYASDNFWTMGDTGP |
| | | CGPCTEVFYDHGADIEGGLPGTPEEDGDRYIEIWNCVFMQFNRQK |
| | | DGTMLPLPAPSVDTGMGLERISAIMQGVHGNYEIDLFVHLMDAAA |
| | | EILEIENQQQSSLKVIADHIRAVSFLIADGVTPSNEGRGYVLRRV |
| | | IRRAVRHGNKLGADSDFFYKMVAPLVAEMGIAYPELKDKQSVIEN |
| | | TIQKEEAQFAKTLAQGLRLLASELEGLQDGDTLSGEAAFKLYDTY |
| | | GFPVDLTADITRERG |
| 26 | 26chAlaRS-1 | MSKSTAEIRQAFLDFFHSKGHQVVASSSLVPHNDPTLLFTNAGMN |
| | | QFKDVFLGLDKRNYSRATTSQRCVRAGGKHNDLENVGYTARHHTF |
| | | FEMLGNFSFGDYFKHDAIQFAWELLTSEKWFALPKERLWVTVYES |
| | | DDEAYEIWEKEVGIPRERIIRIGDNKGAPYASDNFWQMGDTGPCG |
| | | PCTEIFYDHGDHIWGGPPGSPEEDGDRYIEIWNIVFMQFNRQADG |
| | | TMEPLPKPSVDTGMGLERIAAVLQHVNSNYDIDLFRTLIQAVAKV |
| | | TGATDLSNKSLRVIADHIRSCAFLIADGVMPSNENRGYVLRRIIR |
| | | RAVRHGNMLGAKETFFYKLVGPLIDVMGSAGEDLKRQQAQVEQVL |
| | | KTEEEQFARTLERGLALLDEELAGGGSGGGSGGGSGGGSGGGSGG |
| | | GSMDKKPLDVLISATGLWMSRTGTLHKIKHYEISRSKIYIEMACG |
| | | DHLVVNNSRSCRPARAFRYHKYRKTCKRCRVSDEDINNFLTRSTE |
| | | GKTSVKVKVVSEPKVKKAMPKSVSRAPKPLENPVSAKASTDTSRS |
| | | VPSPAKSTPNSPVPTS |
| 27 | 27chAlaRS-2 | MDKKPLDVLISATGLWMSRTGTLHKIKHYEISRSKIYIEMACGDH |
| | | LVVNNSRSCRPARAFRYHKYRKTCKRCRVSDEDINNFLTRSTEGK |
| | | TSVKVKVVSEPKVKKAMPKSVSRAPKPLENPVSAKASTDTSRSVP |
| | | SPAKSTPNSPVPTSASAPALTKSQTDRLEVLLNPKDEISLNSGKP |
| | | FRELESELLSRRKKDLQQIYAEEREGGGSGGMTLDEEYLDITFLT |
| | | ENGFVRKRCPKCGKHFWTADPEREICGDPPCESYSFIGNPVFKKP |
| | | FELDEMREYYLNFFERRGHGRIERYPVVARWRTDIYLTIASIADF |
| | | QPFVTSGVAPPPANPLTISQPCIRLDDLDSVGRTGRHLTLFEMMA |
| | | HHAFNYPGKEIYWKNETVAYCTELLNELGVKKEDIVYKEEPWAGG |
| | | GNAGPCLEAIVGGLEVATLVFMNLEEHPEGDIEIKGARYRKMDNY |
| | | IVDTGYGLERFVWASKGTPTVYDAIFPEVVDTIIDNSNVSFNRED |
| | | ERVRRIVAESSKLAGIMGELRGERLNQLRKSVADTVGVSVEELEG |
| | | IVVPLEKVYSLADHTRCILFMLGDGLVPSNAGAGYLARLMIRRSL |
| | | RLAEELELGLDLYDLVEMHKKILGFEFDVPLSTVQEILELEKERY |
| | | RTTVSKGTRLVERLVERKKKLEKDDLIELYDSHGIPVELAVGIAA |
| | | EKGAEVEMPKDIYAELAKRHS |
| 28 | 28chAlaRS-3 | MDKKPLDVLISATGLWMSRTGTLHKIKHYEISRSKIYIEMACGDH |
| | | LVVNNSRSCRPARAFRYHKYRKTCKRCRVSDEDINNFLTRSTEGK |
| | | TSVKVKVVSEPKVKKAMPKSVSRAPKPLENPVSAKASTDTSRSVP |
| | | SPAKSTPNSPVPTSGGGSGGMTLDEEYLDITFLTENGFVRKRCPK |
| | | CGKHFWTADPEREICGDPPCESYSFIGNPVFKKPFELDEMREYYL |
| | | NFFERRGHGRIERYPVVARWRTDIYLTIASIADFQPFVTSGVAPP |
| | | PANPLTISQPCIRLDDLDSVGRTGRHLTLFEMMAHHAFNYPGKEI |
| | | YWKNETVAYCTELLNELGVKKEDIVYKEEPWAGGGNAGPCLEAIV |
| | | GGLEVATLVFMNLEEHPEGDIEIKGARYRKMDNYIVDTGYGLERF |
| | | VWASKGTPTVYDAIFPEVVDTIIDNSNVSFNREDERVRRIVAESS |
| | | KLAGIMGELRGERLNQLRKSVADTVGVSVEELEGIVVPLEKVYSL |
| | | ADHTRCILFMLGDGLVPSNAGAGYLARLMIRRSLRLAEELELGLD |
| | | LYDLVEMHKKILGFEFDVPLSTVQEILELEKERYRTTVSKGTRLV |
| | | ERLVERKKKLEKDDLIELYDSHGIPVELAVGIAAEKGAEVEMPKD |
| | | IYAELAKRHS |
| 29 | 29chAlaRS-4 | MDKKPLDVLISATGLWMSRTGTLHKIKHYEISRSKIYIEMACGDH |
| | | LVVNNSRSCRPARAFRYHKYRKTCKRCRVSDEDINNFLTRSTEGK |
| | | TSVKVKVVSEPKVGGGSGGMTLDEEYLDITFLTENGFVRKRCPKC |
| | | GKHFWTADPEREICGDPPCESYSFIGNPVFKKPFELDEMREYYLN |
| | | FFERRGHGRIERYPVVARWRTDIYLTIASIADFQPFVTSGVAPPP |
| | | ANPLTISQPCIRLDDLDSVGRTGRHLTLFEMMAHHAFNYPGKEIY |
| | | WKNETVAYCTELLNELGVKKEDIVYKEEPWAGGGNAGPCLEAIVG |
| | | GLEVATLVFMNLEEHPEGDIEIKGARYRKMDNYIVDTGYGLERFV |
| | | WASKGTPTVYDAIFPEVVDTIIDNSNVSFNREDERVRRIVAESSK |
| | | LAGIMGELRGERLNQLRKSVADTVGVSVEELEGIVVPLEKVYSLA |
| | | DHTRCILFMLGDGLVPSNAGAGYLARLMIRRSLRLAEELELGLDL |
| | | YDLVEMHKKILGFEFDVPLSTVQEILELEKERYRTTVSKGTRLVE |
| | | RLVERKKKLEKDDLIELYDSHGIPVELAVGIAAEKGAEVEMPKDI |
| | | YAELAKRHS |
| 30 | 30chAlaRS-5 | MDKKPLDVLISATGLWMSRTGTLHKIKHYEISRSKIYIEMACGDH |
| | | LVVNNSRSCRPARAFRYHKYRKTCKRCRVSDEDINNFLTRSTEGK |
| | | TSVKVKVVSEPKVKKAMPKSVSRAPKPLENPVSAKASTDTSRSVP |
| | | SPAKSTPNSPVPTSASAPALTKSQTDRLEVLLNPKDEISLNSGKP |
| | | FRELESELLSRRKKDLQQIYAEEREGGGSGGAWRGLSHRPLSSEP |
| | | PAAKASAVRAAFLNFFRDRHGHRLVPSASVRPRGDPSLLFVNAGM |
| | | NQFKPIFLGTVDPRSEMAGFRRVANSQKCVRAGGHHNDLEDVGRD |
| | | LSHHTFFEMLGNWAFGGEYFKEEACNMAWELLTQVYGIPEERLWI |
| | | SYFDGDPKAGLDPDLETRDIWLSLGVPASRVLSFGPQENFWEMGD |
| | | TGPCGPCTEIHYDLAGGVGAPQLVELWNLVFMQHNREADGSLQPL |
| | | PQRHVDTGMGLERLVAVLQGKHSTYDTDLFSPLLNAIQQGCRAPP |
| | | YLGRVGVADEGRTDTAYRVVADHIRTLSVCISDGIFPGMSGPPLV |
| | | LRRILRRAVRFSMEILKAPPGFLGSLVPVVVETLGDAYPELQRNS |
| | | AQIANLVSEDEAAFLASLERGRRIIDRTLRTLGPSDMFPAEVAWS |
| | | LSLCGDLGLPLDMVELMLEEKGVQLDS |
| 31 | 31chAlaRS-6 | MVVKFTDSQIQHLMEYGDNDWSEAEFEDAAARDKEFSSQFSKLKS |
| | | ANDKGLKDVIANPRNGGGSGGGGGSGGMTLDEEYLDITFLTENGF |
| | | VRKRCPKCGKHFWTADPEREICGDPPCESYSFIGNPVFKKPFELD |
| | | EMREYYLNFFERRGHGRIERYPVVARWRTDIYLTIASIADFQPFV |
| | | TSGVAPPPANPLTISQPCIRLDDLDSVGRTGRHLTLFEMMAHHAF |
| | | NYPGKEIYWKNETVAYCTELLNELGVKKEDIVYKEEPWAGGGNAG |
| | | PCLEAIVGGLEVATLVFMNLEEHPEGDIEIKGARYRKMDNYIVDT |
| | | GYGLERFVWASKGTPTVYDAIFPEVVDTIIDNSNVSFNREDERVR |
| | | RIVAESSKLAGIMGELRGERLNQLRKSVADTVGVSVEELEGIVVP |
| | | LEKVYSLADHTRCILFMLGDGLVPSNAGAGYLARLMIRRSLRLAE |
| | | ELELGLDLYDLVEMHKKILGFEFDVPLSTVQEILELEKERYRTTV |
| | | SKGTRLVERLVERKKKLEKDDLIELYDSHGIPVELAVGIAAEKGA |
| | | EVEMPKDIYAELAKRHS |
| 32 | 32chAlaRS-7 | MDKKPLDVLISATGLWMSRTGTLHKIKHYEISRSKIYIEMACGDH |
| | | LVVNNSRSCRPARAFRYHKYRKTCKRCRVSDEDINNFLTRSTEGK |
| | | TSVKVKVVSEPKVKKAMPKSVSRAPKPLENPVSAKASTDTSRSVP |
| | | SPAKSTPNSPVPTSASAPALTKSQTDRLEVLLNPKDEISLNSGKP |
| | | FRELESELLSRRKKDLQQIYAEEREGGGSGGLEDEYQLDFFSENG |
| | | FVRKQCSKCGKFYWTRDLDRNTCGDAPCDPYSFIGNPVFNKKFDL |
| | | AEMREYYLKFFEDQGHTRLERYPVVARWRDDIYLTIASIADFQPF |
| | | VTSGQVRPPANPLTISQPCIRLSDLDAVGRSGRHLTTFEMMAHHA |
| | | FNKKGEEIYWKDRTMELCDELFNSLGVDPLAVTYKEEPWAGGGNA |
| | | GPCVEALIGGLEVATLVFMDLEQSKDGTIPIKGENYRKMDNYIVD |
| | | TGYGLERFVWASKGSPTIYDAVFPTIVNELMQLAGLEHQIDDPEY |
| | | ANILSQSARLAGLMDVSEKANLLQLRKQVASSIGTSVEKLSSIME |
| | | PVENIYAITDHTRCLTFMLADGIIPSNVKAGYLARLVLRRTLRMM |
| | | KDMDINTSLSDIVRMHIKNLPEYPEFSEKFDVIEDILAHEEMKFD |
| | | ETLERGRRMIQKSAKHYKSTGEKIPLPTLIEMYASHGIPPEISKE |
| | | AASEMGVEVDLPDNFYSLVAEGHS |
| 33 | 33chAlaRS-8 | MDKKPLDVLISATGLWMSRTGTLHKIKHYEISRSKIYIEMACGDH |
| | | LVVNNSRSCRPARAFRYHKYRKTCKRCRVSDEDINNFLTRSTEGK |
| | | TSVKVKVVSEPKVKKAMPKSVSRAPKPLENPVSAKASTDTSRSVP |
| | | SPAKSTPNSPVPTSASAPALTKSQTDRLEVLLNPKDEISLNSGKP |
| | | FRELESELLSRRKKDLQQIYAEEREGGGSGGLSTKLFETAGFHRK |
| | | QCPMCKSYFWTLRQDQVYCGDQPCVPYGFIGNSPVRVPVESIRDL |
| | | RERFLSFFEKRGHARIKRYPVVARWREDVFLVGASIYDFQPWVTS |
| | | GAVPPPANPLTISQPSIRLTDVDKVGRSGRHLTGFEMMAHHAFNY |
| | | PDKHIYWISETTEYAYEFFTKELGIPEDEITFKESVWEGGGNAGE |
| | | SFEVLVRGLEVATLVFMHYEVKDGKYVELPLKIVDTGYGLERIYW |
| | | MAKGSPTVYDAVFGPFLSAARKKLGVPEPPAHILGKASVYFGQMD |
| | | PEVIGLAKAYDLVAEKIGIDPREFREIMRPQEALYVLADHSRTVS |
| | | WMIADGVIPSNTGAGYLARLLIRRALKSLWIAGIETSLVDLFDLH |
| | | LGFLRDDYPEVWQSRDIILELIDLEEKKYKDVIKSAPAAVRRALE |
| | | KAGGKSLKAEQLVELYASYGIPPEVAQEVARKLGAEAEVPDDFYS |
| | | RLAARHQ |
| 34 | 34chAlaRS-9 | MDKKPLDVLISATGLWMSRTGTLHKIKHYEISRSKIYIEMACGDH |
| | | LVVNNSRSCRPARAFRYHKYRKTCKRCRVSDEDINNFLTRSTEGK |
| | | TSVKVKVVSEPKVKKAMPKSVSRAPKPLENPVSAKASTDTSRSVP |
| | | SPAKSTPNSPVPTSASAPALTKSQTDRLEVLLNPKDEISLNSGKP |
| | | FRELESELLSRRKKDLQQIYAEEREGGGSGGMDMTTRMFKEEGWI |
| | | RKQCPKCGKFFWTLDPDRETCGDPPCDEYSFIGKPGIPKKYTLEE |
| | | MREAFLRFFEKHDHGRVKRFPVLPRWRDDVLLVGASIMDFQPWVI |
| | | SGEADPPANPLTISQPSIRFTDIDNVGITGRHFTIFEMMAHHAFN |
| | | YPGKPIYWMDETVELAFEFFTKELGMKAEDITFKENPWAGGGNAG |
| | | PAFEVLYRGLEVATLVFMQYKKAPENADPSQVVEIKGDYYVPMET |
| | | KVVDTGYGLERLVWMSHGTPTAYDAVLGYVVEPLKRMAGIERIDE |
| | | RILMENSRLAGMFDIEDMGDLRYLRGQVAKRVGISVEELEKAVRP |
| | | YELIYAIADHTKALTFMLADGVIPSNVKAGYLARLLIRKSIRHLR |
| | | ELGLEVPLAEIVAMHIKELSPTFPEFKEMEDVILDIINVEEKRYQ |
| | | ETLKRGSDLVKREIARLKKAGKNEMPLEKLILFYASHGLTPEIVR |
| | | EVAEKEGVKVEIPDNFYTLVAK |
| 35 | 35chAlaRS-10 | MDKKPLDVLISATGLWMSRTGTLHKIKHYEISRSKIYIEMACGDH |
| | | LVVNNSRSCRPARAFRYHKYRKTCKRCRVSDEDINNFLTRSTEGK |
| | | TSVKVKVVSEPKVKKAMPKSVSRAPKPLENPVSAKASTDTSRSVP |
| | | SPAKSTPNSPVPTSASAPALTKSQTDRLEVLLNPKDEISLNSGKP |
| | | FRELESELLSRRKKDLQQIYAEEREGGGSGGMTLDEEYLDITFLT |
| | | ENGFVRKRCPKCGKHFWTADPEREICGDPPCESYSFIGNPVVSQP |
| | | FRSADIRQAFIDFFISKQHTPVASSSLIPHNDPTLLFTNAGMNQF |
| | | KETFLGMEPRDYTRAVTSQKCVRAGGKHNDLDNVGYTARHHTFFE |
| | | MLGNFSFGDYFKQAGIGYIWEFLTSDEWLAIDKNRLYVTIYETDD |
| | | EAFDIWHKDIGIPSERIIRIGDNKGAPYASDNFWTMGDTGPCGPC |
| | | TEVFYDHGADIEGGLPGTPEEDGDRYIEIWNCVFMQFNRQKDGTM |
| | | LPLPAPSVDTGMGLERISAIMQGVHGNYEIDLFVHLMDAAAEILE |
| | | IENQQQSSLKVIADHIRAVSFLIADGVTPSNEGRGYVLRRVIRRA |
| | | VRHGNKLGADSDFFYKMVAPLVAEMGIAYPELKDKQSVIENTIQK |
| | | EEAQFAKTLAQGLRLLASELEGLQDGDTLSGEAAFKLYDTYGFPV |
| | | DLTADITRERGIVIDEAEFDEHMQAQRE |
| 36 | 36chAlaT-1 | GGAGCTATGATCATGTAGATCGAACGGACTX1X2X3AATCCGTTC |
| | | AGCCGGGTTAGATTCCCGGTAGCTCCACCA |
| 37 | 37chAlaT-1_tcg | GGAGCTATGATCATGTAGATCGAACGGACTCGAAATCCGTTCAGC |
| | | CGGGTTAGATTCCCGGTAGCTCCACCA |
| 38 | 38chAlaT-2 | GGActcgTGATCATGTAGATCGAACGGACTX1X2X3AATCCGTTC |
| | | AGCCGGGTTAGATTCCCGGcgagTCCACCA |
| 39 | 39chAlaT-2_TCG | GGActcgTGATCATGTAGATCGAACGGACTCGAAATCCGTTCAGC |
| | | CGGGTTAGATTCCCGGcgagTCCACCA |
| 40 | 40chAlaT-3 | GGactcgGCTCCGGCGAGCAAACGGGTCTX1X2X3AAACCTGTAA |
| | | GCGGGGTTCGACCCCCCGcgagTCCACCA |
| 41 | 41chAlaT-3_TCG | GGactcgGCTCCGGCGAGCAAACGGGTCTCGAAAACCTGTAAGCG |
| | | GGGTTCGACCCCCCGcgagTCCACCA |
| 42 | 42chAlaT-4 | GGACTCGTGATCATGTAGATCGAACGGACTX1X2X3AATCCTTTC |
| | | AGCCGGGTTAGATTCCCGGCGAGTCCACCA |
| 43 | 43chAlaT-4_TCG | GGACTCGTGATCATGTAGATCGAACGGACTCGAAATCCTTTCAGC |
| | | CGGGTTAGATTCCCGGCGAGTCCACCA |
| 44 | 44chAlaT-5 | GGACTCGTGGCCAAGGTGGCCGAACGGACTX1X2X3AATCCGTTC |
| | | AGGGGGGTTCGATTCCCCCGAGTCCACCA |
| 45 | 45chAlaT-5_TCG | GGACTCGTGGCCAAGGTGGCCGAACGGACTCGAAATCCGTTCAGG |
| | | GGGGTTCGATTCCCCCGAGTCCACCA |
| 46 | 46chAlaT-6 | GGACTCGTGGCCAAGGTGGCCGTGTTGACTX1X2X3AATCAACAC |
| | | AGGGGGGTTCGATTCCCCCCGAGTCCACCA |
| 4 | 47chAlaT6_TCG | GGACTCGTGGCCAAGGTGGCCGTGTTGACTCGAAATCAACACAG |
| 7 | | GGGGGTTCGATTCCCCCCGAGTCCACCA |
| 48 | 48chAlaT-7 | GGACTCGTGGCCAAGGTGGCCAGCGGGTCTX1X2X3AAACCTAGC |
| | | CAGACGGGTTCGATTCCTGTCGAGTCCACCA |
| 49 | 49chAlaT-7_TCG | GGACTCGTGGCCAAGGTGGCCAGCGGGTCTCGAAAACCTAGCCAG |
| | | ACGGGTTCGATTCCTGTCGAGTCCACCA |
| 50 | 50linker1 | GGGSGG |
| 51 | 51linker2 | GGGSGGGSGGGSGGGSGGGSGGGS |
| 52 | 52linker3 | APAPAPAPAPAPAPAPAP |
| 53 | 53linker4 | AEAAAKEAAAKEAAAKA |
| 54 | 54linker5 | GGGSGGGSGGGS |
| 55 | 55linker6 | GGGSGGGSGGGSGGGSGG |
| 56 | 56AibRS-1 | MSKSTAEIRQAFLDFFHSKGHQVVASSSLVPHNDPTLLFTNAGMN |
| | | QFKDVFLGLDKRNYSRATTSQRCVRAGGKHNDLENVGYTARHHTF |
| | | FEMLGNFSFGDYFKHDAIQFAWELLTSEKWFALPKERLWVTVYES |
| | | DDEAYEIWEKEVGIPRERIIRIGDNKGAPYASDNFHQMGDTGPCG |
| | | PCTEIFYDHGDHIWGGPPGSPEEDGDRYIEIWNIGFMQFNRQADG |
| | | TMEPLPKPSVDVGMGLERIAAVLQHVNSNYDIDLFRTLIQAVAKV |
| | | TGATDLSNKSLRVIADHIRSCAFLIADGVMPSNENRGYVLRRIIR |
| | | RAVRHGNMLGAKETFFYKLVGPLIDVMGSAGEDLKRQQAQVEQVL |
| | | KTEEEQFARTLERGLALLDEELAGGGSGGGSGGGSGGGSGGGSGG |
| | | GSMDKKPLDVLISATGLWMSRTGTLHKIKHYEISRSKIYIEMACG |
| | | DHLVVNNSRSCRPARAFRYHKYRKTCKRCRVSDEDINNFLTRSTE |
| | | GKTSVKVKVVSEPKVKKAMPKSVSRAPKPLENPVSAKASTDTSRS |
| | | VPSPAKSTPNSPVPTS |
| 57 | 57AibRS-2 | MDKKPLDVLISATGLWMSRTGTLHKIKHYEISRSKIYIEMACGDH |
| | | LVVNNSRSCRPARAFRYHKYRKTCKRCRVSDEDINNFLTRSTEGK |
| | | TSVKVKVVSEPKVKKAMPKSVSRAPKPLENPVSAKASTDTSRSVP |
| | | SPAKSTPNSPVPTSASAPALTKSQTDRLEVLLNPKDEISLNSGKP |
| | | FRELESELLSRRKKDLQQIYAEEREGGGSGGMTLDEEYLDITFLT |
| | | ENGFVRKRCPKCGKHFWTADPEREICGDPPCESYSFIGNPVFKKP |
| | | FELDEMREYYLNFFERRGHGRIERYPVVARWRTDIYLTIASIADF |
| | | QPFVTSGVAPPPANPLTISQPCIRLDDLDSVGRTGRHLTLFEMMA |
| | | HHAFNYPGKEIYWKNETVAYCTELLNELGVKKEDIVYKEEPHAGG |
| | | GNAGPCLEAIVGGLEVATLGFMNLEEHPEGDIEIKGARYRKMDNY |
| | | IVDVGYGLERFVWASKGTPTVYDAIFPEVVDTIIDNSNVSFNRED |
| | | ERVRRIVAESSKLAGIMGELRGERLNQLRKSVADTVGVSVEELEG |
| | | IVVPLEKVYSLADHTRCILFMLGDGLVPSNAGAGYLARLMIRRSL |
| | | RLAEELELGLDLYDLVEMHKKILGFEFDVPLSTVQEILELEKERY |
| | | RTTVSKGTRLVERLVERKKKLEKDDLIELYASHGIPVELAVGIAA |
| | | EKGAEVEMPKDIYAELAKRHS |
| 58 | 58AibRS-3 | MVVKFTDSQIQHLMEYGDNDWSEAEFEDAAARDKEFSSQFSKLKS |
| | | ANDKGLKDVIANPRNGGGSGGMTLDEEYLDITFLTENGFVRKRCP |
| | | KCGKHFWTADPEREICGDPPCESYSFIGNPVFKKPFELDEMREYY |
| | | LNFFERRGHGRIERYPVVARWRTDIYLTIASIADFQPFVTSGVAP |
| | | PPANPLTISQPCIRLDDLDSVGRTGRHLTLFEMMAHHAFNYPGKE |
| | | IYWKNETVAYCTELLNELGVKKEDIVYKEEPHAGGGNAGPCLEAI |
| | | VGGLEVATLGFMNLEEHPEGDIEIKGARYRKMDNYIVDVGYGLER |
| | | FVWASKGTPTVYDAIFPEVVDTIIDNSNVSFNREDERVRRIVAES |
| | | SKLAGIMGELRGERLNQLRKSVADTVGVSVEELEGIVVPLEKVYS |
| | | LADHTRCILFMLGDGLVPSAAGAGYLARLMIRRSLRLAEELELGL |
| | | DLYDLVEMHKKILGFEFDVPLSTVQEILELEKERYRTTVSKGTRL |
| | | VERLVERKKKLEKDDLIELYASHGIPVALAVGIAAEKGAEVEMPK |
| | | DIYAELAKRHS |
| 59 | 59AibRS-4 | MDKKPLDVLISATGLWMSRTGTLHKIKHYEISRSKIYIEMACGDH |
| | | LVVNNSRSCRPARAFRYHKYRKTCKRCRVSDEDINNFLTRSTEGK |
| | | TSVKVKVVSEPKVKKAMPKSVSRAPKPLENPVSAKASTDTSRSVP |
| | | SPAKSTPNSPVPTSASAPALTKSQTDRLEVLLNPKDEISLNSGKP |
| | | FRELESELLSRRKKDLQQIYAEEREGGGSGGMTLDEEYLDITFLT |
| | | ENGFVRKRCPKCGKHFWTADPEREICGDPPCESYSFIGNPVFKKP |
| | | FELDEMREYYLNFFERRGHGRIERYPVVARWRTDIYLTIASIADF |
| | | QPFVTSGVAPPPANPLTISQPCIRLDDLDSVGRTGRHLTLFEMMA |
| | | HHAFNYPGKEIYWKNETVAYCTELLNELGVKKEDIVYKEEPHAGG |
| | | GNAGPCLEAIVGGLEVATLGFMNLEEHPEGDIEIKGARYRKMDNY |
| | | IVDVGYGLERFVWASKGTPTVYDAIFPEVVDTIIDNSNVSFNRED |
| | | ERVRRIVAESSKLAGIMGELRGERLNQLRKSVADTVGVSVEELEG |
| | | IVVPLEKVYSLADHTRCILFMLGDGLVPSAAGAGYLARLMIRRSL |
| | | RLAEELELGLDLYDLVEMHKKILGFEFDVPLSTVQEILELEKERY |
| | | RTTVSKGTRLVERLVERKKKLEKDDLIELYASHGIPVALAVGIAA |
| | | EKGAEVEMPKDIYAELAKRHS |
| 60 | 60AibRS-5 | MDKKPLDVLISATGLWMSRTGTLHKIKHYEISRSKIYIEMACGDH |
| | | LVVNNSRSCRPARAFRYHKYRKTCKRCRVSDEDINNFLTRSTEGK |
| | | TSVKVKVVSEPKVKKAMPKSVSRAPKPLENPVSAKASTDTSRSVP |
| | | SPAKSTPNSPVPTSASAPALTKSQTDRLEVLLNPKDEISLNSGKP |
| | | FRELESELLSRRKKDLQQIYAEEREGGGSGGMTLDEEYLDITFLT |
| | | ENGFVRKRCPKCGKHFWSADPEREICGDPPCESYSFIGNPAFKNP |
| | | FELDEMREYYLNFFERRGHGRIERYPVVAWWRTDIYLTIASIADF |
| | | QPFVTSGVAPPPANPLTISQPCIRLDDLDCVGRTGRHLTLFEMMA |
| | | HHAFNYPGKEIYWKNETVAYCTELLNELGVKKEDIVYKEELHAGG |
| | | GNAGPCLEAIVGGLEVATLGFMNLEEHPEGDIEIKGARYRKMDNY |
| | | IVDVGYGLERFVWASKGTPTVYDAIFPEVVDTIIDNSNVSFNRED |
| | | DRVRRIVAESSKLAGIMGELRGERLNRLRKSVADIVGVSVEELEG |
| | | IVVPLEKVYSLADHTRCILFMLGDGLVPSKAGAGYLARLMIRRSL |
| | | RLAEELELGLDLYDLVEMHKKILGFEFDVPLSTVQEILELEKERY |
| | | RTTVSKGTRLVERLVERKKKLEKDDLIELYCSHGIPVALAVGIAA |
| | | EKGAEVEMPKDIYAELAKRHS |
| 61 | 61AibRS-6 | MDKKPLDVLISATGLWMSRTGTLHKIKHYEISRSKIYIEMACGDH |
| | | LVVNNSRSCRPARAFRYHKYRKTCKRCRVSDEDINNFLTRSTEGK |
| | | TSVKVKVVSEPKVKKAMPKSVSRAPKPLENPVSAKASTDTSRSVP |
| | | SPAKSTPNSPVPTSASAPALTKSQTDRLEVLLNPKDEISLNSGKP |
| | | FRELESELLSRRKKDLQQIYAEEREGGGSGGMTLDEEYLDITFLT |
| | | ENGFVRKRCPKCGKHFWSADPEREICGDPPCESYSFIGNPAFKNP |
| | | FELDEMREYYLNFFERRGHGRIERYPVVAWWRTDIYLTIASIADF |
| | | QPFVTSGVAPPPANPLTISQPCIRLDDLDCVGRTGRHLTLFEMMA |
| | | HHAFNYPGKEIYWKNETVAYCTELLNELGVKKEDIVYKEELFAGG |
| | | GNAGPCLEAIVGGLEVATLGFMNLEEHPEGDIEIKGARYRKMDNY |
| | | IVDVGYGLERFVWASKGTPTVYDAIFPEVVDTIIDNSNVSFNRED |
| | | DRVRRIVAESSKLAGIMGELRGERLNRLRKSVADIVGVSVEELEG |
| | | IVVPLEKVYSLADHTRCILFMLGDGLVPSKAGAGYLARLMIRRSL |
| | | RLAEELELGLDLYDLVEMHKKILGFEFDVPLSTVQEILELEKERY |
| | | RTTVSKGTRLVERLVERKKKLEKDDLIELYCSHGIPVALAVGIAA |
| | | EKGAEVEMPKDIYAELAKRHS |
| 62 | 62AibRS-7 | MDKKPLDVLISATGLWMSRTGTLHKIKHYEISRSKIYIEMACGDH |
| | | LVVNNSRSCRPARAFRYHKYRKTCKRCRVSDEDINNFLTRSTEGK |
| | | TSVKVKVVSEPKVKKAMPKSVSRAPKPLENPVSAKASTDTSRSVP |
| | | SPAKSTPNSPVPTSASAPALTKSQTDRLEVLLNPKDEISLNSGKP |
| | | FRELESELLSRRKKDLQQIYAEEREGGGSGGMTLDEEYLDITFLT |
| | | ENGFVRKRCPKCGKHFWSADPEREICGDPPCESYSFIGNPAFKNP |
| | | FELDEMREYYLNFFERRGHGRIERYPVVAWWRTDIYLTIASIADF |
| | | QPFVTSGVAPPPANPLTISQPCIRLDDLDCVGRTGRHLTLFEMMA |
| | | HHAFNYPGKEIYWKNETVAYCTELLNELGVKKEDIVYKEELYAGG |
| | | GNAGPCLEAIVGGLEVATLGFMNLEEHPEGDIEIKGARYRKMDNY |
| | | IVDVGYGLERFVWASKGTPTVYDAIFPEVVDTIIDNSNVSFNRED |
| | | DRVRRIVAESSKLAGIMGELRGERLNRLRKSVADIVGVSVEELEG |
| | | IVVPLEKVYSLADHTRCILFMLGDGLVPSKAGAGYLARLMIRRSL |
| | | RLAEELELGLDLYDLVEMHKKILGFEFDVPLSTVQEILELEKERY |
| | | RTTVSKGTRLVERLVERKKKLEKDDLIELYCSHGIPVALAVGIAA |
| | | EKGAEVEMPKDIYAELAKRHS |
| 63 | 63AibRS-8 | MDKKPLDVLISATGLWMSRTGTLHKIKHYEISRSKIYIEMACGDH |
| | | LVVNNSRSCRPARAFRYHKYRKTCKRCRVSDEDINNFLTRSTEGK |
| | | TSVKVKVVSEPKVKKAMPKSVSRAPKPLENPVSAKASTDTSRSVP |
| | | SPAKSTPNSPVPTSASAPALTKSQTDRLEVLLNPKDEISLNSGKP |
| | | FRELESELLSRRKKDLQQIYAEEREGGGSGGMTLDEEYLDITFLT |
| | | ENGFVRKRCPKCGKHFWSADPEREICGDPPCESYSFIGNPAFKNP |
| | | FELDEMREYYLNFFERRGHGRIERYPVVAWWRTDIYLTIASIADF |
| | | QPFVTSGVAPPPANPLTISQPCIRLDDLDCVGRTGRHLTLFEMMA |
| | | HHAFNYPGKEIYWKNETVAYCTELLNELGVKKEDIVYKEELHAGG |
| | | GNAGPCLEAIVGGLEVATLGFMNLEEHPEGDIEIKGARYRKMDNY |
| | | IVDTGYGLERFVWASKGTPTVYDAIFPEVVDTIIDNSNVSFNRED |
| | | DRVRRIVAESSKLAGIMGELRGERLNRLRKSVADIVGVSVEELEG |
| | | IVVPLEKVYSLADHTRCILFMLGDGLVPSKAGAGYLARLMIRRSL |
| | | RLAEELELGLDLYDLVEMHKKILGFEFDVPLSTVQEILELEKERY |
| | | RTTVSKGTRLVERLVERKKKLEKDDLIELYCSHGIPVALAVGIAA |
| | | EKGAEVEMPKDIYAELAKRHS |
| 64 | 64AibRS-9 | MDKKPLDVLISATGLWMSRTGTLHKIKHYEISRSKIYIEMACGDH |
| | | LVVNNSRSCRPARAFRYHKYRKTCKRCRVSDEDINNFLTRSTEGK |
| | | TSVKVKVVSEPKVKKAMPKSVSRAPKPLENPVSAKASTDTSRSVP |
| | | SPAKSTPNSPVPTSASAPALTKSQTDRLEVLLNPKDEISLNSGKP |
| | | FRELESELLSRRKKDLQQIYAEEREGGGSGGMTLDEEYLDITFLT |
| | | ENGFVRKRCPKCGKHFWSADPEREICGDPPCESYSFIGNPAFKNP |
| | | FELDEMREYYLNFFERRGHGRIERYPVVAWWRTDIYLTIASIADF |
| | | QPFVTSGVAPPPANPLTISQPCIRLDDLDCVGRTGRHLTLFEMMA |
| | | HHAFNYPGKEIYWKNETVAYCTELLNELGVKKEDIVYKEELFAGG |
| | | GNAGPCLEAIVGGLEVATLGFMNLEEHPEGDIEIKGARYRKMDNY |
| | | IVDTGYGLERFVWASKGTPTVYDAIFPEVVDTIIDNSNVSFNRED |
| | | DRVRRIVAESSKLAGIMGELRGERLNRLRKSVADIVGVSVEELEG |
| | | IVVPLEKVYSLADHTRCILFMLGDGLVPSKAGAGYLARLMIRRSL |
| | | RLAEELELGLDLYDLVEMHKKILGFEFDVPLSTVQEILELEKERY |
| | | RTTVSKGTRLVERLVERKKKLEKDDLIELYCSHGIPVALAVGIAA |
| | | EKGAEVEMPKDIYAELAKRHS |
| 65 | 65AibRS-10 | MDKKPLDVLISATGLWMSRTGTLHKIKHYEISRSKIYIEMACGDH |
| | | LVVNNSRSCRPARAFRYHKYRKTCKRCRVSDEDINNFLTRSTEGK |
| | | TSVKVKVVSEPKVKKAMPKSVSRAPKPLENPVSAKASTDTSRSVP |
| | | SPAKSTPNSPVPTSASAPALTKSQTDRLEVLLNPKDEISLNSGKP |
| | | FRELESELLSRRKKDLQQIYAEEREGGGSGGMTLDEEYLDITFLT |
| | | ENGFVRKRCPKCGKHFWSADPEREICGDPPCESYSFIGNPAFKNP |
| | | FELDEMREYYLNFFERRGHGRIERYPVVAWWRTDIYLTIASIADF |
| | | QPFVTSGVAPPPANPLTISQPCIRLDDLDCVGRTGRHLTLFEMMA |
| | | HHAFNYPGKEIYWKNETVAYCTELLNELGVKKEDIVYKEELYAGG |
| | | GNAGPCLEAIVGGLEVATLGFMNLEEHPEGDIEIKGARYRKMDNY |
| | | IVDTGYGLERFVWASKGTPTVYDAIFPEVVDTIIDNSNVSFNRED |
| | | DRVRRIVAESSKLAGIMGELRGERLNRLRKSVADIVGVSVEELEG |
| | | IVVPLEKVYSLADHTRCILFMLGDGLVPSKAGAGYLARLMIRRSL |
| | | RLAEELELGLDLYDLVEMHKKILGFEFDVPLSTVQEILELEKERY |
| | | RTTVSKGTRLVERLVERKKKLEKDDLIELYCSHGIPVALAVGIAA |
| | | EKGAEVEMPKDIYAELAKRHS |
| 66 | 66AibRS-11 | MDKKPLDVLISATGLWMSRTGTLHKIKHYEISRSKIYIEMACGDH |
| | | LVVNNSRSCRPARAFRYHKYRKTCKRCRVSDEDINNFLTRSTEGK |
| | | TSVKVKVVSEPKVKKAMPKSVSRAPKPLENPVSAKASTDTSRSVP |
| | | SPAKSTPNSPVPTSASAPALTKSQTDRLEVLLNPKDEISLNSGKP |
| | | FRELESELLSRRKKDLQQIYAEEREGGGSGGMTLDEEYLDITFLT |
| | | ENGFVRKRCPKCGKHFWSADPEREICGDPPCESYSFIGNPAFKNP |
| | | FELDEMREYYLNFFERRGHGRIERYPVVAWWRTDIYLTIASIADF |
| | | QPFVTSGVAPPPANPLTISQPCIRLDDLDCVGRTGRHLTLFEMMA |
| | | HHAFNYPGKEIYWKNETVAYCTELLNELGVKKEDIVYKEELLAGG |
| | | GNAGPCLEAIVGGLEVATLGFMNLEEHPEGDIEIKGARYRKMDNY |
| | | IVDVGYGLERFVWASKGTPTVYDAIFPEVVDTIIDNSNVSFNRED |
| | | DRVRRIVAESSKLAGIMGELRGERLNRLRKSVADIVGVSVEELEG |
| | | IVVPLEKVYSLADHTRCILFMLGDGLVPSKAGAGYLARLMIRRSL |
| | | RLAEELELGLDLYDLVEMHKKILGFEFDVPLSTVQEILELEKERY |
| | | RTTVSKGTRLVERLVERKKKLEKDDLIELYCSHGIPVALAVGIAA |
| | | EKGAEVEMPKDIYAELAKRHS |
| 67 | 67AibRS-12 | MDKKPLDVLISATGLWMSRTGTLHKIKHYEISRSKIYIEMACGDH |
| | | LVVNNSRSCRPARAFRYHKYRKTCKRCRVSDEDINNFLTRSTEGK |
| | | TSVKVKVVSEPKVKKAMPKSVSRAPKPLENPVSAKASTDTSRSVP |
| | | SPAKSTPNSPVPTSASAPALTKSQTDRLEVLLNPKDEISLNSGKP |
| | | FRELESELLSRRKKDLQQIYAEEREGGGSGGMTLDEEYLDITFLT |
| | | ENGFVRKRCPKCGKHFWSADPEREICGDPPCESYSFIGNPAFKNP |
| | | FELDEMREYYLNFFERRGHGRIERYPVVAWWRTDIYLTIASIADF |
| | | QPFVTSGVAPPPANPLTISQPCIRLDDLDCVGRTGRHLTLFEMMA |
| | | HHAFNYPGKEIYWKNETVAYCTELLNELGVKKEDIVYKEELIAGG |
| | | GNAGPCLEAIVGGLEVATLGFMNLEEHPEGDIEIKGARYRKMDNY |
| | | IVDVGYGLERFVWASKGTPTVYDAIFPEVVDTIIDNSNVSFNRED |
| | | DRVRRIVAESSKLAGIMGELRGERLNRLRKSVADIVGVSVEELEG |
| | | IVVPLEKVYSLADHTRCILFMLGDGLVPSKAGAGYLARLMIRRSL |
| | | RLAEELELGLDLYDLVEMHKKILGFEFDVPLSTVQEILELEKERY |
| | | RTTVSKGTRLVERLVERKKKLEKDDLIELYCSHGIPVALAVGIAA |
| | | EKGAEVEMPKDIYAELAKRHS |
| 68 | 68AibRS-13 | MDKKPLDVLISATGLWMSRTGTLHKIKHYEISRSKIYIEMACGDH |
| | | LVVNNSRSCRPARAFRYHKYRKTCKRCRVSDEDINNFLTRSTEGK |
| | | TSVKVKVVSEPKVKKAMPKSVSRAPKPLENPVSAKASTDTSRSVP |
| | | SPAKSTPNSPVPTSASAPALTKSQTDRLEVLLNPKDEISLNSGKP |
| | | FRELESELLSRRKKDLQQIYAEEREGGGSGGMTLDEEYLDITFLT |
| | | ENGFVRKRCPKCGKHFWSADPEREICGDPPCESYSFIGNPAFKNP |
| | | FELDEMREYYLNFFERRGHGRIERYPVVAWWRTDIYLTIASIADF |
| | | QPFVTSGVAPPPANPLTISQPCIRLDDLDCVGRTGRHLTLFEMMA |
| | | HHAFNYPGKEIYWKNETVAYCTELLNELGVKKEDIVYKEELMAGG |
| | | GNAGPCLEAIVGGLEVATLGFMNLEEHPEGDIEIKGARYRKMDNY |
| | | IVDVGYGLERFVWASKGTPTVYDAIFPEVVDTIIDNSNVSFNRED |
| | | DRVRRIVAESSKLAGIMGELRGERLNRLRKSVADIVGVSVEELEG |
| | | IVVPLEKVYSLADHTRCILFMLGDGLVPSKAGAGYLARLMIRRSL |
| | | RLAEELELGLDLYDLVEMHKKILGFEFDVPLSTVQEILELEKERY |
| | | RTTVSKGTRLVERLVERKKKLEKDDLIELYCSHGIPVALAVGIAA |
| | | EKGAEVEMPKDIYAELAKRHS |
| 69 | 69AibRS-14 | MDKKPLDVLISATGLWMSRTGTLHKIKHYEISRSKIYIEMACGDH |
| | | LVVNNSRSCRPARAFRYHKYRKTCKRCRVSDEDINNFLTRSTEGK |
| | | TSVKVKVVSEPKVKKAMPKSVSRAPKPLENPVSAKASTDTSRSVP |
| | | SPAKSTPNSPVPTSASAPALTKSQTDRLEVLLNPKDEISLNSGKP |
| | | FRELESELLSRRKKDLQQIYAEEREGGGSGGMTLDEEYLDITFLT |
| | | ENGFVRKRCPKCGKHFWSADPEREICGDPPCESYSFIGNPAFKNP |
| | | FELDEMREYYLNFFERRGHGRIERYPVVAWWRTDIYLTIASIADF |
| | | QPFVTSGVAPPPANPLTISQPCIRLDDLDCVGRTGRHLTLFEMMA |
| | | HHAFNYPGKEIYWKNETVAYCTELLNELGVKKEDIVYKEELHAGG |
| | | GNAGPCLEAIVGGLEVATLAFMNLEEHPEGDIEIKGARYRKMDNY |
| | | IVDVGYGLERFVWASKGTPTVYDAIFPEVVDTIIDNSNVSFNRED |
| | | DRVRRIVAESSKLAGIMGELRGERLNRLRKSVADIVGVSVEELEG |
| | | IVVPLEKVYSLADHTRCILFMLGDGLVPSKAGAGYLARLMIRRSL |
| | | RLAEELELGLDLYDLVEMHKKILGFEFDVPLSTVQEILELEKERY |
| | | RTTVSKGTRLVERLVERKKKLEKDDLIELYCSHGIPVALAVGIAA |
| | | EKGAEVEMPKDIYAELAKRHS |
| 70 | 70AibRS-15 | MDKKPLDVLISATGLWMSRTGTLHKIKHYEISRSKIYIEMACGDH |
| | | LVVNNSRSCRPARAFRYHKYRKTCKRCRVSDEDINNFLTRSTEGK |
| | | TSVKVKVVSEPKVKKAMPKSVSRAPKPLENPVSAKASTDTSRSVP |
| | | SPAKSTPNSPVPTSASAPALTKSQTDRLEVLLNPKDEISLNSGKP |
| | | FRELESELLSRRKKDLQQIYAEEREGGGSGGMTLDEEYLDITFLT |
| | | ENGFVRKRCPKCGKHFWSADPEREICGDPPCESYSFIGNPAFKNP |
| | | FELDEMREYYLNFFERRGHGRIERYPVVAWWRTDIYLTIASIADF |
| | | QPFVTSGVAPPPANPLTISQPCIRLDDLDCVGRTGRHLTLFEMMA |
| | | HHAFNYPGKEIYWKNETVAYCTELLNELGVKKEDIVYKEELFAGG |
| | | GNAGPCLEAIVGGLEVASLGFMNLEEHPEGDIEIKGARYRKMDNY |
| | | IVDVGYGLERFVWASKGTPTVYDAIFPEVVDTIIDNSNVSFNRED |
| | | DRVRRIVAESSKLAGIMGELRGERLNRLRKSVADIVGVSVEELEG |
| | | IVVPLEKVYSLADHTRCILFMLGDGLVPSKAGAGYLARLMIRRSL |
| | | RLAEELELGLDLYDLVEMHKKILGFEFDVPLSTVQEILELEKERY |
| | | RTTVSKGTRLVERLVERKKKLEKDDLIELYCSHGIPVALAVGIAA |
| | | EKGAEVEMPKDIYAELAKRHS |
| 71 | 71AibRS-16 | MDKKPLDVLISATGLWMSRTGTLHKIKHYEISRSKIYIEMACGDH |
| | | LVVNNSRSCRPARAFRYHKYRKTCKRCRVSDEDINNFLTRSTEGK |
| | | TSVKVKVVSEPKVKKAMPKSVSRAPKPLENPVSAKASTDTSRSVP |
| | | SPAKSTPNSPVPTSASAPALTKSQTDRLEVLLNPKDEISLNSGKP |
| | | FRELESELLSRRKKDLQQIYAEEREGGGSGGMTLDEEYLDITFLT |
| | | ENGFVRKRCPKCGKHFWSADPEREICGDPPCESYSFIGNPAFKNP |
| | | FELDEMREYYLNFFERRGHGRIERYPVVAWWRTDIYLTIASIADF |
| | | QPFVTSGVAPPPANPLTISQPCIRLDDLDCVGRTGRHLTLFEMMA |
| | | HHAFNYPGKEIYWKNETVAYCTELLNELGVKKEDIVYKEELFAGG |
| | | GNAGPCLEAIVGGLEVATLGFMNLEEHPEGDIEIKGARYRKMDNY |
| | | IVDLGYGLERFVWASKGTPTVYDAIFPEVVDTIIDNSNVSFNRED |
| | | DRVRRIVAESSKLAGIMGELRGERLNRLRKSVADIVGVSVEELEG |
| | | IVVPLEKVYSLADHTRCILFMLGDGLVPSKAGAGYLARLMIRRSL |
| | | RLAEELELGLDLYDLVEMHKKILGFEFDVPLSTVQEILELEKERY |
| | | RTTVSKGTRLVERLVERKKKLEKDDLIELYCSHGIPVALAVGIAA |
| | | EKGAEVEMPKDIYAELAKRHS |
| 72 | 72AibRS-17 | MDKKPLDVLISATGLWMSRTGTLHKIKHYEISRSKIYIEMACGDH |
| | | LVVNNSRSCRPARAFRYHKYRKTCKRCRVSDEDINNFLTRSTEGK |
| | | TSVKVKVVSEPKVKKAMPKSVSRAPKPLENPVSAKASTDTSRSVP |
| | | SPAKSTPNSPVPTSASAPALTKSQTDRLEVLLNPKDEISLNSGKP |
| | | FRELESELLSRRKKDLQQIYAEEREGGGSGGMTLDEEYLDITFLT |
| | | ENGFVRKRCPKCGKHFWSADPEREICGDPPCESYSFIGNPAFKNP |
| | | FELDEMREYYLNFFERRGHGRIERYPVVAWWRTDIYLTIASIADF |
| | | QPFVTSGVAPPPANPLTISQPCIRLDDLDCVGRTGRHLTLFEMMA |
| | | HHAFNYPGKEIYWKNETVAYCTELLNELGVKKEDIVYKEELFAGG |
| | | GNAGPCLEAIVGGLEVATLGFMNLEEHPEGDIEIKGARYRKMDNY |
| | | IVEVGYGLERFVWASKGTPTVYDAIFPEVVDTIIDNSNVSFNRED |
| | | DRVRRIVAESSKLAGIMGELRGERLNRLRKSVADIVGVSVEELEG |
| | | IVVPLEKVYSLADHTRCILFMLGDGLVPSKAGAGYLARLMIRRSL |
| | | RLAEELELGLDLYDLVEMHKKILGFEFDVPLSTVQEILELEKERY |
| | | RTTVSKGTRLVERLVERKKKLEKDDLIELYCSHGIPVALAVGIAA |
| | | EKGAEVEMPKDIYAELAKRHS |
| 73 | 73AibRS-18 | MDKKPLDVLISATGLWMSRTGTLHKIKHYEISRSKIYIEMACGDH |
| | | LVVNNSRSCRPARAFRYHKYRKTCKRCRVSDEDINNFLTRSTEGK |
| | | TSVKVKVVSEPKVKKAMPKSVSRAPKPLENPVSAKASTDTSRSVP |
| | | SPAKSTPNSPVPTSASAPALTKSQTDRLEVLLNPKDEISLNSGKP |
| | | FRELESELLSRRKKDLQQIYAEEREGGGSGGMTLDEEYLDITFLT |
| | | ENGFVRKRCPKCGKHFWSADPEREICGDPPCESYSFIGNPAFKNP |
| | | FELDEMREYYLNFFERRGHGRIERYPVVAWWRTDIYLTIASIADF |
| | | QPFVTSGVAPPPANPLTISQPCIRLDDLDCVGRTGRHLTLFEMMA |
| | | HHAFNYPGKEIYWKNETVAYCTELLNELGVKKEDIVYKEELHAGG |
| | | GNAGPCLEAIVGGLEVAGLGFMNLEEHPEGDIEIKGARYRKMDNY |
| | | IVDVGYGLERFVWASKGTPTVYDAIFPEVVDTIIDNSNVSFNRED |
| | | DRVRRIVAESSKLAGIMGELRGERLNRLRKSVADIVGVSVEELEG |
| | | IVVPLEKVYSLADHTRCILFMLGDGLVPSKAGAGYLARLMIRRSL |
| | | RLAEELELGLDLYDLVEMHKKILGFEFDVPLSTVQEILELEKERY |
| | | RTTVSKGTRLVERLVERKKKLEKDDLIELYCSHGIPVALAVGIAA |
| | | EKGAEVEMPKDIYAELAKRHS |
| 74 | 74AibRS-19 | MSKSTAEIRQAFLDFFHSKGHQVVASSSLVPHNDPTLLFTNAGMN |
| | | QFKDVFLGLDKRNYSRATTSQRCVRAGGKHNDLENVGYTARHHTF |
| | | FEMLGNFSFGDYFKHDAIQFAWELLTSEKWFALPKERLWVTVYES |
| | | DDEAYEIWEKEVGIPRERIIRIGDNKGAPYASDNFHIMGDTGPCG |
| | | PCTEIFYDHGDHIWGGPPGSPEEDGDRYIEIWNIGFIQFNRQADG |
| | | TMEPLPKPSVDTGMGLERIAAVLQHVNSNYDIDLFRTLIQAVAKV |
| | | TGATDLSNKSLRVIADHIRSCAFLIADGVMPSNENRGYVLRRIIR |
| | | RAVRHGNMLGAKETFFYKLVGPLIDVMGSAGEDLKRQQAQVEQVL |
| | | KTEEEQFARTLERGLALLDEELAGGGSGGGSGGGSGGGSGGGSGG |
| | | GSMDKKPLDVLISATGLWMSRTGTLHKIKHYEISRSKIYIEMACG |
| | | DHLVVNNSRSCRPARAFRYHKYRKTCKRCRVSDEDINNFLTRSTE |
| | | GKTSVKVKVVSEPKVKKAMPKSVSRAPKPLENPVSAKASTDTSRS |
| | | VPSPAKSTPNSPVPTS |
| 75 | 75AibRS-20 | MDKKPLDVLISATGLWMSRTGTLHKIKHYEISRSKIYIEMACGDH |
| | | LVVNNSRSCRPARAFRYHKYRKTCKRCRVSDEDINNFLTRSTEGK |
| | | TSVKVKVVSEPKVKKAMPKSVSRAPKPLENPVSAKASTDTSRSVP |
| | | SPAKSTPNSPVPTSASAPALTKSQTDRLEVLLNPKDEISLNSGKP |
| | | FRELESELLSRRKKDLQQIYAEEREGGGSGGMTLDEEYLDITFLT |
| | | ENGFVRKRCPKCGKHFWTADPEREICGDPPCESYSFIGNPVFKKP |
| | | FELDMSKSTAEIRQAFLDFFHSKGHQVVASSSLVPHNDPTLLFTN |
| | | AGMNQFKDVFLGLDKRNYSRATTSQRCVRAGGKHNDLENVGYTAR |
| | | HHTFFEMLGNFSFGDYFKHDAIQFAWELLTSEKWFALPKERLWVT |
| | | VYESDDEAYEIWEKEVGIPRERIIRIGDNKGAPYASDNFHQMGDT |
| | | GPCGPCTEIFYDHGDHIWGGPPGSPEEDGDRYIEIWNIGFMQFNR |
| | | QADGTMEPLPKPSVDVGMGLERIAAVLQHVNSNYDIDLFRTLIQA |
| | | VAKVTGATDLSNKSLRVIADHIRSCAFLIADGVMPSKENRGYVLR |
| | | RIIRRAVRHGNMLGAKETFFYKLVGPLIDVMGSAGEDLKRQQAQV |
| | | EQVLKTEEEQFARTLERGLALLDEELAKLSGDTLDGETAFRLYRT |
| | | YGFPVALTADVCRERNIKVDEAGFEAAMEEQRRRAREASGF |
| 76 | 76sema | H(Aib)EGTFTSDVSSYLEGQAAKEFIAWLVRGRG |
| 77 | 77semadmeC | H(dmeC)EGTFTSDVSSYLEGQAAKEFIAWLVRGRG |
| 78 | 78semadmeS | H(dmeS)EGTFTSDVSSYLEGQAAKEFIAWLVRGRG |
| 79 | 79semaD-iso | H(D-isoVal)EGTFTSDVSSYLEGQAAKEFIAWLVRGRG |
| | Val | |
| 80 | 80Tir | Y(Aib)EGTFTSDYSI(Aib)LDKIAQKAFVQWLIAGGPSSGAPP |
| | | PS |
| 81 | 81sumo- | atgggcagcagccatcatcatcatcatcacagcagcggcctggtg |
| | glp1- | ccgcgcggcagcATGTCGGACTCAGAAGTCAATCAAGAAGCTAAG |
| | 8tag | CCAGAGGTCAAGCCAGAAGTCAAGCCTGAGACTCACATCAATTTA |
| | | AAGGTGTCCGATGGATCTTCAGAGATCTTCTTCAAGATCAAAAAG |
| | | ACCACTCCTTTAAGAAGGCTGATGGAAGCGTTCGCTAAAAGACAG |
| | | GGTAAGGAAATGGACTCCTTAAGATTCTTGTACGACGGTATTAGA |
| | | ATTCAAGCTGATCAGACCCCTGAAGATTTGGACATGGAGGATAAC |
| | | GATATTATTGAGGCTCACAGAGAACAGATTGGTGGACATtagGAA |
| | | GGCACCTTTACCAGCGATGTGAGCAGCTATCTGGAAGGCCAGGCG |
| | | GCGaaaGAATTTATTGCGTGGCTGGTGAGGGGCCGCGGC |
| 82 | 82mae-glp- | atgaaaattaaaaccggcgcgcgcattctggcgctgagcgcgctg |
| | 8tag | accaccatgatgtttagcgctagcgcgctggccCATGCCGAAGGC |
| | | ACGTTCACCTCGGATGTTTCAAGCTATTTAGAGGGCCAGGCAGCG |
| | | aaaGAATTTATTGCGTGGCTGGTGCGTGGTCGCGGA |
| 83 | 832*sema | ATGggcCACCACCACCATGAAGAAGCGGAGGCAGAAGCTCGCGGT |
| | | AAACGTCACTAGGAAGGCACGTTCACCTCTGACGTAAGCTCTTAC |
| | | CTGGAGGGTCAGGCGGCCAAAGAATTTATTGCGTGGCTGGTTCGT |
| | | GGTCGTGGTAAACGTCACTAGGAAGGTACCTTCACTAGCGACGTT |
| | | TCTTCCTATCTGGAGGGCCAGGCGGCAAAAGAATTCATTGCGTGG |
| | | CTGGTACGTGGCCGTGGT |
| 84 | 843*sema | ATGggcCACCACCACCACGAAGAAGCTGAAGCTGAAGCGCGCGGT |
| | | AAACGCCACTAGGAGGGTACCTTCACCTCTGACGTGTCCTCTTAC |
| | | CTGGAAGGTCAGGCAGCCAAAGAGTTCATCGCTTGGCTGGTACGT |
| | | GGTCGCGGTAAACGTCATTAGGAAGGCACCTTCACCTCTGATGTA |
| | | TCTTCCTATCTGGAGGGTCAAGCTGCTAAGGAATTCATTGCGTGG |
| | | CTGGTTCGTGGTCGTGGTAAACGTCACTAGGAAGGTACCTTCACT |
| | | AGCGATGTGTCCAGCTACCTGGAAGGTCAGGCGGCTAAAGAGTTC |
| | | ATCGCATGGCTGGTACGTGGCCGCGGT |
| 85 | 854*sema | ATGggcCACCACCACCATGAAGAAGCTGAAGCTGAAGCGCGCGGT |
| | | AAACGTCACTAGGAGGGTACCTTCACCTCTGATGTTTCCTCTTAC |
| | | CTGGAGGGTCAGGCGGCCAAAGAATTCATTGCTTGGCTGGTTCGT |
| | | GGTCGCGGCAAACGTCACTAGGAAGGTACCTTCACCAGCGATGTC |
| | | TCCAGCTATCTGGAAGGCCAGGCGGCAAAAGAATTTATCGCCTGG |
| | | CTGGTGCGTGGTCGCGGTAAACGTCACTAGGAAGGTACGTTTACT |
| | | AGCGACGTTAGCTCCTACCTGGAAGGCCAGGCGGCGAAAGAATTT |
| | | ATCGCGTGGCTGGTGCGTGGTCGTGGCAAACGCCATTAGGAAGGC |
| | | ACGTTCACTTCTGATGTTAGCTCCTATCTGGAAGGTCAGGCAGCG |
| | | AAAGAATTCATTGCGTGGCTGGTACGCGGCCGTGGT |
| 86 | TD1 | MDKKPLDVLISATGLWMSRTGTLHKIKHYEISRSKIYIEMACGDH |
| | | LVVNNSRSCRPARAFRYHKYRKTCKRCRVSDEDINNFLTRSTEGK |
| | | TSVKVKVVSEPKV |
| 87 | TD2 | MDKKPLDVLISATGLWMSRTGTLHKIKHYEISRSKIYIEMACGDH |
| | | LVVNNSRSCRPARAFRYHKYRKTCKRCRVSDEDINNFLTRSTEGK |
| | | TSVKVKVVSEPKVKKAMPKSVSRAPKPLENPVSAKASTDTSRSVP |
| | | SPAKSTPNSPVPTS |
| 88 | CD1 | AWRGLSHRPLSSEPPAAKASAVRAAFLNFFRDRHGHRLVPSASVR |
| | | PRGDPSLLFVNAGMNQFKPIFLGTVDPRSEMAGFRRVANSQKCVR |
| | | AGGHHNDLEDVGRDLSHHTFFEMLGNWAFGGEYFKEEACNMAWEL |
| | | LTQVYGIPEERLWISYFDGDPKAGLDPDLETRDIWLSLGVPASRV |
| | | LSFGPQENFWEMGDTGPCGPCTEIHYDLAGGVGAPQLVELWNLVF |
| | | MQHNREADGSLQPLPQRHVDTGMGLERLVAVLQGKHSTYDTDLFS |
| | | PLLNAIQQGCRAPPYLGRVGVADEGRTDTAYRVVADHIRTLSVCI |
| | | SDGIFPGMSGPPLVLRRILRRAVRFSMEILKAPPGFLGSLVPVVV |
| | | ETLGDAYPELQRNSAQIANLVSEDEAAFLASLERGRRIIDRTLRT |
| | | LGPSDMFPAEVAWSLSLCGDLGLPLDMVELMLEEKGVQLDS |
| 89 | D2C | LEDEYQLDFFSENGFVRKQCSKCGKFYWTRDLDRNTCGDAPCDPY |
| | | SFIGNPVFNKKFDLAEMREYYLKFFEDQGHTRLERYPVVARWRDD |
| | | IYLTIASIADFQPFVTSGQVRPPANPLTISQPCIRLSDLDAVGRS |
| | | GRHLTTFEMMAHHAFNKKGEEIYWKDRTMELCDELFNSLGVDPLA |
| | | VTYKEEPWAGGGNAGPCVEALIGGLEVATLVFMDLEQSKDGTIPI |
| | | KGENYRKMDNYIVDTGYGLERFVWASKGSPTIYDAVFPTIVNELM |
| | | QLAGLEHQIDDPEYANILSQSARLAGLMDVSEKANLLQLRKQVAS |
| | | SIGTSVEKLSSIMEPVENIYAITDHTRCLTFMLADGIIPSNVKAG |
| | | YLARLVLRRTLRMMKDMDINTSLSDIVRMHIKNLPEYPEFSEKFD |
| | | VIEDILAHEEMKFDETLERGRRMIQKSAKHYKSTGEKIPLPTLIE |
| | | MYASHGIPPEISKEAASEMGVEVDLPDNFYSLVAEGHS |
| 90 | CD3 | LSTKLFETAGFHRKQCPMCKSYFWTLRQDQVYCGDQPCVPYGFIG |
| | | NSPVRVPVESIRDLRERFLSFFEKRGHARIKRYPVVARWREDVFL |
| | | VGASIYDFQPWVTSGAVPPPANPLTISQPSIRLTDVDKVGRSGRH |
| | | LTGFEMMAHHAFNYPDKHIYWISETTEYAYEFFTKELGIPEDEIT |
| | | FKESVWEGGGNAGESFEVLVRGLEVATLVFMHYEVKDGKYVELPL |
| | | KIVDTGYGLERIYWMAKGSPTVYDAVFGPFLSAARKKLGVPEPPA |
| | | HILGKASVYFGQMDPEVIGLAKAYDLVAEKIGIDPREFREIMRPQ |
| | | EALYVLADHSRTVSWMIADGVIPSNTGAGYLARLLIRRALKSLWI |
| | | AGIETSLVDLFDLHLGFLRDDYPEVWQSRDIILELIDLEEKKYKD |
| | | VIKSAPAAVRRALEKAGGKSLKAEQLVELYASYGIPPEVAQEVAR |
| | | KLGAEAEVPDDFYSRLAARHQ |
| 91 | CD4 | MDMTTRMFKEEGWIRKQCPKCGKFFWTLDPDRETCGDPPCDEYSF |
| | | IGKPGIPKKYTLEEMREAFLRFFEKHDHGRVKRFPVLPRWRDDVL |
| | | LVGASIMDFQPWVISGEADPPANPLTISQPSIRFTDIDNVGITGR |
| | | HFTIFEMMAHHAFNYPGKPIYWMDETVELAFEFFTKELGMKAEDI |
| | | TFKENPWAGGGNAGPAFEVLYRGLEVATLVFMQYKKAPENADPSQ |
| | | VVEIKGDYYVPMETKVVDTGYGLERLVWMSHGTPTAYDAVLGYVV |
| | | EPLKRMAGIERIDERILMENSRLAGMFDIEDMGDLRYLRGQVAKR |
| | | VGISVEELEKAVRPYELIYAIADHTKALTFMLADGVIPSNVKAGY |
| | | LARLLIRKSIRHLRELGLEVPLAEIVAMHIKELSPTFPEFKEMED |
| | | VILDIINVEEKRYQETLKRGSDLVKREIARLKKAGKNEMPLEKLI |
| | | LFYASHGLTPEIVREVAEKEGVKVEIPDNFYTLVAK |
| 92 | CD5 | MTLDEEYLDITFLTENGFVRKRCPKCGKHFWTADPEREICGDPPC |
| | | ESYSFIGNPVVSQPFRSADIRQAFIDFFISKQHTPVASSSLIPHN |
| | | DPTLLFTNAGMNQFKETFLGMEPRDYTRAVTSQKCVRAGGKHNDL |
| | | DNVGYTARHHTFFEMLGNFSFGDYFKQAGIGYIWEFLTSDEWLAI |
| | | DKNRLYVTIYETDDEAFDIWHKDIGIPSERIIRIGDNKGAPYASD |
| | | NFWTMGDTGPCGPCTEVFYDHGADIEGGLPGTPEEDGDRYIEIWN |
| | | CVFMQFNRQKDGTMLPLPAPSVDTGMGLERISAIMQGVHGNYEID |
| | | LFVHLMDAAAEILEIENQQQSSLKVIADHIRAVSFLIADGVTPSN |
| | | EGRGYVLRRVIRRAVRHGNKLGADSDFFYKMVAPLVAEMGIAYPE |
| | | LKDKQSVIENTIQKEEAQFAKTLAQGLRLLASELEGLQDGDTLSG |
| | | EAAFKLYDTYGFPVDLTADITRERGIVIDEAEFDEHMQAQRE |
| 93 | CD6 | MSKSTAEIRQAFLDFFHSKGHQVVASSSLVPHNDPTLLFTNAGMN |
| | | QFKDVFLGLDKRNYSRATTSQRCVRAGGKHNDLENVGYTARHHTF |
| | | FEMLGNFSFGDYFKHDAIQFAWELLTSEKWFALPKERLWVTVYES |
| | | DDEAYEIWEKEVGIPRERIIRIGDNKGAPYASDNFHQMGDTGPCG |
| | | PCTEIFYDHGDHIWGGPPGSPEEDGDRYIEIWNIGFMQFNRQADG |
| | | TMEPLPKPSVDVGMGLERIAAVLQHVNSNYDIDLFRTLIQAVAKV |
| | | TGATDLSNKSLRVIADHIRSCAFLIADGVMPSNENRGYVLRRIIR |
| | | RAVRHGNMLGAKETFFYKLVGPLIDVMGSAGEDLKRQQAQVEQVL |
| | | KTEEEQFARTLERGLALLDEELA |
| 94 | CD7 | MTLDEEYLDITFLTENGFVRKRCPKCGKHFWTADPEREICGDPPC |
| | | ESYSFIGNPVFKKPFELDEMREYYLNFFERRGHGRIERYPVVARW |
| | | RTDIYLTIASIADFQPFVTSGVAPPPANPLTISQPCIRLDDLDSV |
| | | GRTGRHLTLFEMMAHHAFNYPGKEIYWKNETVAYCTELLNELGVK |
| | | KEDIVYKEEPHAGGGNAGPCLEAIVGGLEVATLGFMNLEEHPEGD |
| | | IEIKGARYRKMDNYIVDVGYGLERFVWASKGTPTVYDAIFPEVVD |
| | | TIIDNSNVSFNREDERVRRIVAESSKLAGIMGELRGERLNQLRKS |
| | | VADTVGVSVEELEGIVVPLEKVYSLADHTRCILFMLGDGLVPSNA |
| | | GAGYLARLMIRRSLRLAEELELGLDLYDLVEMHKKILGFEFDVPL |
| | | STVQEILELEKERYRTTVSKGTRLVERLVERKKKLEKDDLIELYA |
| | | SHGIPVELAVGIAAEKGAEVEMPKDIYAELAKRHS |
| 95 | CD8 | MTLDEEYLDITFLTENGFVRKRCPKCGKHFWTADPEREICGDPPC |
| | | ESYSFIGNPVFKKPFELDEMREYYLNFFERRGHGRIERYPVVARW |
| | | RTDIYLTIASIADFQPFVTSGVAPPPANPLTISQPCIRLDDLDSV |
| | | GRTGRHLTLFEMMAHHAFNYPGKEIYWKNETVAYCTELLNELGVK |
| | | KEDIVYKEEPHAGGGNAGPCLEAIVGGLEVATLGFMNLEEHPEGD |
| | | IEIKGARYRKMDNYIVDVGYGLERFVWASKGTPTVYDAIFPEVVD |
| | | TIIDNSNVSFNREDERVRRIVAESSKLAGIMGELRGERLNQLRKS |
| | | VADTVGVSVEELEGIVVPLEKVYSLADHTRCILFMLGDGLVPSAA |
| | | GAGYLARLMIRRSLRLAEELELGLDLYDLVEMHKKILGFEFDVPL |
| | | STVQEILELEKERYRTTVSKGTRLVERLVERKKKLEKDDLIELYA |
| | | SHGIPVALAVGIAAEKGAEVEMPKDIYAELAKRHS |
| 96 | CD9 | MTLDEEYLDITFLTENGFVRKRCPKCGKHFWSADPEREICGDPPC |
| | | ESYSFIGNPAFKNPFELDEMREYYLNFFERRGHGRIERYPVVAWW |
| | | RTDIYLTIASIADFQPFVTSGVAPPPANPLTISQPCIRLDDLDCV |
| | | GRTGRHLTLFEMMAHHAFNYPGKEIYWKNETVAYCTELLNELGVK |
| | | KEDIVYKEELHAGGGNAGPCLEAIVGGLEVATLGFMNLEEHPEGD |
| | | IEIKGARYRKMDNYIVDVGYGLERFVWASKGTPTVYDAIFPEVVD |
| | | TIIDNSNVSFNREDDRVRRIVAESSKLAGIMGELRGERLNRLRKS |
| | | VADIVGVSVEELEGIVVPLEKVYSLADHTRCILFMLGDGLVPSKA |
| | | GAGYLARLMIRRSLRLAEELELGLDLYDLVEMHKKILGFEFDVPL |
| | | STVQEILELEKERYRTTVSKGTRLVERLVERKKKLEKDDLIELYC |
| | | SHGIPVALAVGIAAEKGAEVEMPKDIYAELAKRHS |
| 97 | CD10 | MTLDEEYLDITFLTENGFVRKRCPKCGKHFWSADPEREICGDPPC |
| | | ESYSFIGNPAFKNPFELDEMREYYLNFFERRGHGRIERYPVVAWW |
| | | RTDIYLTIASIADFQPFVTSGVAPPPANPLTISQPCIRLDDLDCV |
| | | GRTGRHLTLFEMMAHHAFNYPGKEIYWKNETVAYCTELLNELGVK |
| | | KEDIVYKEELFAGGGNAGPCLEAIVGGLEVATLGFMNLEEHPEGD |
| | | IEIKGARYRKMDNYIVDVGYGLERFVWASKGTPTVYDAIFPEVVD |
| | | TIIDNSNVSFNREDDRVRRIVAESSKLAGIMGELRGERLNRLRKS |
| | | VADIVGVSVEELEGIVVPLEKVYSLADHTRCILFMLGDGLVPSKA |
| | | GAGYLARLMIRRSLRLAEELELGLDLYDLVEMHKKILGFEFDVPL |
| | | STVQEILELEKERYRTTVSKGTRLVERLVERKKKLEKDDLIELYC |
| | | SHGIPVALAVGIAAEKGAEVEMPKDIYAELAKRHS |
| 98 | CD11 | MTLDEEYLDITFLTENGFVRKRCPKCGKHFWSADPEREICGDPPC |
| | | ESYSFIGNPAFKNPFELDEMREYYLNFFERRGHGRIERYPVVAWW |
| | | RTDIYLTIASIADFQPFVTSGVAPPPANPLTISQPCIRLDDLDCV |
| | | GRTGRHLTLFEMMAHHAFNYPGKEIYWKNETVAYCTELLNELGVK |
| | | KEDIVYKEELYAGGGNAGPCLEAIVGGLEVATLGFMNLEEHPEGD |
| | | IEIKGARYRKMDNYIVDVGYGLERFVWASKGTPTVYDAIFPEVVD |
| | | TIIDNSNVSFNREDDRVRRIVAESSKLAGIMGELRGERLNRLRKS |
| | | VADIVGVSVEELEGIVVPLEKVYSLADHTRCILFMLGDGLVPSKA |
| | | GAGYLARLMIRRSLRLAEELELGLDLYDLVEMHKKILGFEFDVPL |
| | | STVQEILELEKERYRTTVSKGTRLVERLVERKKKLEKDDLIELYC |
| | | SHGIPVALAVGIAAEKGAEVEMPKDIYAELAKRHS |
| 99 | CD12 | MTLDEEYLDITFLTENGFVRKRCPKCGKHFWSADPEREICGDPPC |
| | | ESYSFIGNPAFKNPFELDEMREYYLNFFERRGHGRIERYPVVAWW |
| | | RTDIYLTIASIADFQPFVTSGVAPPPANPLTISQPCIRLDDLDCV |
| | | GRTGRHLTLFEMMAHHAFNYPGKEIYWKNETVAYCTELLNELGVK |
| | | KEDIVYKEELHAGGGNAGPCLEAIVGGLEVATLGFMNLEEHPEGD |
| | | IEIKGARYRKMDNYIVDTGYGLERFVWASKGTPTVYDAIFPEVVD |
| | | TIIDNSNVSFNREDDRVRRIVAESSKLAGIMGELRGERLNRLRKS |
| | | VADIVGVSVEELEGIVVPLEKVYSLADHTRCILFMLGDGLVPSKA |
| | | GAGYLARLMIRRSLRLAEELELGLDLYDLVEMHKKILGFEFDVPL |
| | | STVQEILELEKERYRTTVSKGTRLVERLVERKKKLEKDDLIELYC |
| | | SHGIPVALAVGIAAEKGAEVEMPKDIYAELAKRHS |
| 100 | CD13 | MTLDEEYLDITFLTENGFVRKRCPKCGKHFWSADPEREICGDPPC |
| | | ESYSFIGNPAFKNPFELDEMREYYLNFFERRGHGRIERYPVVAWW |
| | | RTDIYLTIASIADFQPFVTSGVAPPPANPLTISQPCIRLDDLDCV |
| | | GRTGRHLTLFEMMAHHAFNYPGKEIYWKNETVAYCTELLNELGVK |
| | | KEDIVYKEELFAGGGNAGPCLEAIVGGLEVATLGFMNLEEHPEGD |
| | | IEIKGARYRKMDNYIVDTGYGLERFVWASKGTPTVYDAIFPEVVD |
| | | TIIDNSNVSFNREDDRVRRIVAESSKLAGIMGELRGERLNRLRKS |
| | | VADIVGVSVEELEGIVVPLEKVYSLADHTRCILFMLGDGLVPSKA |
| | | GAGYLARLMIRRSLRLAEELELGLDLYDLVEMHKKILGFEFDVPL |
| | | STVQEILELEKERYRTTVSKGTRLVERLVERKKKLEKDDLIELYC |
| | | SHGIPVALAVGIAAEKGAEVEMPKDIYAELAKRHS |
| 101 | CD14 | MTLDEEYLDITFLTENGFVRKRCPKCGKHFWSADPEREICGDPPC |
| | | ESYSFIGNPAFKNPFELDEMREYYLNFFERRGHGRIERYPVVAWW |
| | | RTDIYLTIASIADFQPFVTSGVAPPPANPLTISQPCIRLDDLDCV |
| | | GRTGRHLTLFEMMAHHAFNYPGKEIYWKNETVAYCTELLNELGVK |
| | | KEDIVYKEELYAGGGNAGPCLEAIVGGLEVATLGFMNLEEHPEGD |
| | | IEIKGARYRKMDNYIVDTGYGLERFVWASKGTPTVYDAIFPEVVD |
| | | TIIDNSNVSFNREDDRVRRIVAESSKLAGIMGELRGERLNRLRKS |
| | | VADIVGVSVEELEGIVVPLEKVYSLADHTRCILFMLGDGLVPSKA |
| | | GAGYLARLMIRRSLRLAEELELGLDLYDLVEMHKKILGFEFDVPL |
| | | STVQEILELEKERYRTTVSKGTRLVERLVERKKKLEKDDLIELYC |
| | | SHGIPVALAVGIAAEKGAEVEMPKDIYAELAKRHS |
| 102 | CD15 | MTLDEEYLDITFLTENGFVRKRCPKCGKHFWSADPEREICGDPPC |
| | | ESYSFIGNPAFKNPFELDEMREYYLNFFERRGHGRIERYPVVAWW |
| | | RTDIYLTIASIADFQPFVTSGVAPPPANPLTISQPCIRLDDLDCV |
| | | GRTGRHLTLFEMMAHHAFNYPGKEIYWKNETVAYCTELLNELGVK |
| | | KEDIVYKEELLAGGGNAGPCLEAIVGGLEVATLGFMNLEEHPEGD |
| | | IEIKGARYRKMDNYIVDVGYGLERFVWASKGTPTVYDAIFPEVVD |
| | | TIIDNSNVSFNREDDRVRRIVAESSKLAGIMGELRGERLNRLRKS |
| | | VADIVGVSVEELEGIVVPLEKVYSLADHTRCILFMLGDGLVPSKA |
| | | GAGYLARLMIRRSLRLAEELELGLDLYDLVEMHKKILGFEFDVPL |
| | | STVQEILELEKERYRTTVSKGTRLVERLVERKKKLEKDDLIELYC |
| | | SHGIPVALAVGIAAEKGAEVEMPKDIYAELAKRHS |
| 103 | CD16 | MTLDEEYLDITFLTENGFVRKRCPKCGKHFWSADPEREICGDPPC |
| | | ESYSFIGNPAFKNPFELDEMREYYLNFFERRGHGRIERYPVVAWW |
| | | RTDIYLTIASIADFQPFVTSGVAPPPANPLTISQPCIRLDDLDCV |
| | | GRTGRHLTLFEMMAHHAFNYPGKEIYWKNETVAYCTELLNELGVK |
| | | KEDIVYKEELIAGGGNAGPCLEAIVGGLEVATLGFMNLEEHPEGD |
| | | IEIKGARYRKMDNYIVDVGYGLERFVWASKGTPTVYDAIFPEVVD |
| | | TIIDNSNVSFNREDDRVRRIVAESSKLAGIMGELRGERLNRLRKS |
| | | VADIVGVSVEELEGIVVPLEKVYSLADHTRCILFMLGDGLVPSKA |
| | | GAGYLARLMIRRSLRLAEELELGLDLYDLVEMHKKILGFEFDVPL |
| | | STVQEILELEKERYRTTVSKGTRLVERLVERKKKLEKDDLIELYC |
| | | SHGIPVALAVGIAAEKGAEVEMPKDIYAELAKRHS |
| 104 | CD17 | MTLDEEYLDITFLTENGFVRKRCPKCGKHFWSADPEREICGDPPC |
| | | ESYSFIGNPAFKNPFELDEMREYYLNFFERRGHGRIERYPVVAWW |
| | | RTDIYLTIASIADFQPFVTSGVAPPPANPLTISQPCIRLDDLDCV |
| | | GRTGRHLTLFEMMAHHAFNYPGKEIYWKNETVAYCTELLNELGVK |
| | | KEDIVYKEELMAGGGNAGPCLEAIVGGLEVATLGFMNLEEHPEGD |
| | | IEIKGARYRKMDNYIVDVGYGLERFVWASKGTPTVYDAIFPEVVD |
| | | TIIDNSNVSFNREDDRVRRIVAESSKLAGIMGELRGERLNRLRKS |
| | | VADIVGVSVEELEGIVVPLEKVYSLADHTRCILFMLGDGLVPSKA |
| | | GAGYLARLMIRRSLRLAEELELGLDLYDLVEMHKKILGFEFDVPL |
| | | STVQEILELEKERYRTTVSKGTRLVERLVERKKKLEKDDLIELYC |
| | | SHGIPVALAVGIAAEKGAEVEMPKDIYAELAKRHS |
| 105 | CD18 | MTLDEEYLDITFLTENGFVRKRCPKCGKHFWSADPEREICGDPPC |
| | | ESYSFIGNPAFKNPFELDEMREYYLNFFERRGHGRIERYPVVAWW |
| | | RTDIYLTIASIADFQPFVTSGVAPPPANPLTISQPCIRLDDLDCV |
| | | GRTGRHLTLFEMMAHHAFNYPGKEIYWKNETVAYCTELLNELGVK |
| | | KEDIVYKEELHAGGGNAGPCLEAIVGGLEVATLAFMNLEEHPEGD |
| | | IEIKGARYRKMDNYIVDVGYGLERFVWASKGTPTVYDAIFPEVVD |
| | | TIIDNSNVSFNREDDRVRRIVAESSKLAGIMGELRGERLNRLRKS |
| | | VADIVGVSVEELEGIVVPLEKVYSLADHTRCILFMLGDGLVPSKA |
| | | GAGYLARLMIRRSLRLAEELELGLDLYDLVEMHKKILGFEFDVPL |
| | | STVQEILELEKERYRTTVSKGTRLVERLVERKKKLEKDDLIELYC |
| | | SHGIPVALAVGIAAEKGAEVEMPKDIYAELAKRHS |
| 106 | CD19 | MTLDEEYLDITFLTENGFVRKRCPKCGKHFWSADPEREICGDPPC |
| | | ESYSFIGNPAFKNPFELDEMREYYLNFFERRGHGRIERYPVVAWW |
| | | RTDIYLTIASIADFQPFVTSGVAPPPANPLTISQPCIRLDDLDCV |
| | | GRTGRHLTLFEMMAHHAFNYPGKEIYWKNETVAYCTELLNELGVK |
| | | KEDIVYKEELFAGGGNAGPCLEAIVGGLEVASLGFMNLEEHPEGD |
| | | IEIKGARYRKMDNYIVDVGYGLERFVWASKGTPTVYDAIFPEVVD |
| | | TIIDNSNVSFNREDDRVRRIVAESSKLAGIMGELRGERLNRLRKS |
| | | VADIVGVSVEELEGIVVPLEKVYSLADHTRCILFMLGDGLVPSKA |
| | | GAGYLARLMIRRSLRLAEELELGLDLYDLVEMHKKILGFEFDVPL |
| | | STVQEILELEKERYRTTVSKGTRLVERLVERKKKLEKDDLIELYC |
| | | SHGIPVALAVGIAAEKGAEVEMPKDIYAELAKRHS |
| 107 | CD20 | MTLDEEYLDITFLTENGFVRKRCPKCGKHFWSADPEREICGDPPC |
| | | ESYSFIGNPAFKNPFELDEMREYYLNFFERRGHGRIERYPVVAWW |
| | | RTDIYLTIASIADFQPFVTSGVAPPPANPLTISQPCIRLDDLDCV |
| | | GRTGRHLTLFEMMAHHAFNYPGKEIYWKNETVAYCTELLNELGVK |
| | | KEDIVYKEELFAGGGNAGPCLEAIVGGLEVATLGFMNLEEHPEGD |
| | | IEIKGARYRKMDNYIVDLGYGLERFVWASKGTPTVYDAIFPEVVD |
| | | TIIDNSNVSFNREDDRVRRIVAESSKLAGIMGELRGERLNRLRKS |
| | | VADIVGVSVEELEGIVVPLEKVYSLADHTRCILFMLGDGLVPSKA |
| | | GAGYLARLMIRRSLRLAEELELGLDLYDLVEMHKKILGFEFDVPL |
| | | STVQEILELEKERYRTTVSKGTRLVERLVERKKKLEKDDLIELYC |
| | | SHGIPVALAVGIAAEKGAEVEMPKDIYAELAKRHS |
| 108 | CD21 | MTLDEEYLDITFLTENGFVRKRCPKCGKHFWSADPEREICGDPPC |
| | | ESYSFIGNPAFKNPFELDEMREYYLNFFERRGHGRIERYPVVAWW |
| | | RTDIYLTIASIADFQPFVTSGVAPPPANPLTISQPCIRLDDLDCV |
| | | GRTGRHLTLFEMMAHHAFNYPGKEIYWKNETVAYCTELLNELGVK |
| | | KEDIVYKEELFAGGGNAGPCLEAIVGGLEVATLGFMNLEEHPEGD |
| | | IEIKGARYRKMDNYIVEVGYGLERFVWASKGTPTVYDAIFPEVVD |
| | | TIIDNSNVSFNREDDRVRRIVAESSKLAGIMGELRGERLNRLRKS |
| | | VADIVGVSVEELEGIVVPLEKVYSLADHTRCILFMLGDGLVPSKA |
| | | GAGYLARLMIRRSLRLAEELELGLDLYDLVEMHKKILGFEFDVPL |
| | | STVQEILELEKERYRTTVSKGTRLVERLVERKKKLEKDDLIELYC |
| | | SHGIPVALAVGIAAEKGAEVEMPKDIYAELAKRHS |
| 109 | CD22 | MTLDEEYLDITFLTENGFVRKRCPKCGKHFWSADPEREICGDPPC |
| | | ESYSFIGNPAFKNPFELDEMREYYLNFFERRGHGRIERYPVVAWW |
| | | RTDIYLTIASIADFQPFVTSGVAPPPANPLTISQPCIRLDDLDCV |
| | | GRTGRHLTLFEMMAHHAFNYPGKEIYWKNETVAYCTELLNELGVK |
| | | KEDIVYKEELHAGGGNAGPCLEAIVGGLEVAGLGFMNLEEHPEGD |
| | | IEIKGARYRKMDNYIVDVGYGLERFVWASKGTPTVYDAIFPEVVD |
| | | TIIDNSNVSFNREDDRVRRIVAESSKLAGIMGELRGERLNRLRKS |
| | | VADIVGVSVEELEGIVVPLEKVYSLADHTRCILFMLGDGLVPSKA |
| | | GAGYLARLMIRRSLRLAEELELGLDLYDLVEMHKKILGFEFDVPL |
| | | STVQEILELEKERYRTTVSKGTRLVERLVERKKKLEKDDLIELYC |
| | | SHGIPVALAVGIAAEKGAEVEMPKDIYAELAKRHS |
| 110 | CD23 | MSKSTAEIRQAFLDFFHSKGHQVVASSSLVPHNDPTLLFTNAGMN |
| | | QFKDVFLGLDKRNYSRATTSQRCVRAGGKHNDLENVGYTARHHTF |
| | | FEMLGNFSFGDYFKHDAIQFAWELLTSEKWFALPKERLWVTVYES |
| | | DDEAYEIWEKEVGIPRERIIRIGDNKGAPYASDNFHIMGDTGPCG |
| | | PCTEIFYDHGDHIWGGPPGSPEEDGDRYIEIWNIGFIQFNRQADG |
| | | TMEPLPKPSVDTGMGLERIAAVLQHVNSNYDIDLFRTLIQAVAKV |
| | | TGATDLSNKSLRVIADHIRSCAFLIADGVMPSNENRGYVLRRIIR |
| | | RAVRHGNMLGAKETFFYKLVGPLIDVMGSAGEDLKRQQAQVEQVL |
| | | KTEEEQFARTLERGLALLDEELA |
| 111 | CD24 | MTLDEEYLDITFLTENGFVRKRCPKCGKHFWTADPEREICGDPPC |
| | | ESYSFIGNPVFKKPFELDMSKSTAEIRQAFLDFFHSKGHQVVASS |
| | | SLVPHNDPTLLFTNAGMNQFKDVFLGLDKRNYSRATTSQRCVRAG |
| | | GKHNDLENVGYTARHHTFFEMLGNFSFGDYFKHDAIQFAWELLTS |
| | | EKWFALPKERLWVTVYESDDEAYEIWEKEVGIPRERIIRIGDNKG |
| | | APYASDNFHQMGDTGPCGPCTEIFYDHGDHIWGGPPGSPEEDGDR |
| | | YIEIWNIGFMQFNRQADGTMEPLPKPSVDVGMGLERIAAVLQHVN |
| | | SNYDIDLFRTLIQAVAKVTGATDLSNKSLRVIADHIRSCAFLIAD |
| | | GVMPSKENRGYVLRRIIRRAVRHGNMLGAKETFFYKLVGPLIDVM |
| | | GSAGEDLKRQQAQVEQVLKTEEEQFARTLERGLALLDEELAKLSG |
| | | DTLDGETAFRLYRTYGFPVALTADVCRERNIKVDEAGFEAAMEEQ |
| | | RRRAREASGF |
| 112 | Maz | H(Aib)QGTFTSDYSKYLDEKKACEFVEWLLEGGPSSG |
| 113 | Pep1 | H(Aib)QGTFTSDCSKYLDERAAQDFVQWLLDGGPSSGAPPPS |
| 114 | Pep2 | H(Aib)QGTFTSDCSKYLDERAAQDFVQWLLEGGPSSGAPPPS |
| 115 | Pep3 | H(Aib)QGTFTSDCSKYLDERAAQDFVQWLLDGGPSSG |
| 116 | Amycretin | H(Aib)EGTFTSDVSSYLEEQAAREFIAWLVRGRKGGGGEASELS |
| | | TAALGRLSAELHELATLPRTETGSGSP |
| 117 | P30 | Y(Aib)EGTFTSDYSILLEEQAAREFIEWLLAGGPSSGAGQAPGQ |
| | | APLASHLSTAQTQRLSAELHKLATLPRTETGSGSP |

What is claimed is:

1. A noncanonical amino acid genetic encoding system for synthesizing polypeptides comprising noncanonical amino acids (ncAA) comprising a chimeric alanyl-tRNA synthetase (chAlaRS) or a nucleotide sequence encoding the chimeric alanyl-tRNA synthetase,
wherein said chimeric alanyl-tRNA synthetase comprises a tRNA binding domain (TD) and a catalytic domain (CD), and
the tRNA binding domain (TD) and the catalytic domain (CD) of said chimeric alanyl-tRNA synthetase are derived from different aminoacyl-tRNA synthetases.

2. The noncanonical amino acid genetic encoding system according to claim 1,
wherein the tRNA binding domain (TD) of said chimeric alanyl-tRNA synthetase is derived from a pyrrolysyl-tRNA synthetase (PylRS), wherein the tRNA binding domain (TD) comprises the sequence as shown in any one of SEQ ID NO: 1-15, 86-87 or a variant thereof.

3. The noncanonical amino acid genetic encoding system according to claim 1,
wherein the catalytic domain (CD) of said chimeric alanyl-tRNA synthetase is derived from an alanyl-tRNA synthetase (AlaRS), and the catalytic domain (CD) comprises the sequence as shown in any one of SEQ ID NOs: 16-25, 88-111 or a variant thereof.

4. The noncanonical amino acid genetic encoding system according to claim 1,
wherein the chimeric alanyl-tRNA synthetase comprises the sequence as shown in any one of SEQ ID NOs: 26-35, 56-75 or a variant thereof.

5. The noncanonical amino acid genetic encoding system according to claim 1, further comprising a recombinant tRNA or a nucleotide sequence encoding the recombinant tRNA, wherein the recombinant tRNA comprises a first portion and a second portion, wherein the first portion matches the tRNA binding domain (TD) and the second portion matches the catalytic domain (CD).

6. The noncanonical amino acid genetic encoding system according to claim 5,
wherein an acceptor stem of the recombinant tRNA has an A3-U70 or G3-U70 mutation compared to a wild type tRNA.

7. The noncanonical amino acid genetic encoding system according to claim 5,
wherein the recombinant tRNA comprises the sequence as shown in any one of SEQ ID NOs: 36-49 or a variant thereof.

8. The noncanonical amino acid genetic encoding system according to claim 1,
wherein said chimeric alanyl-tRNA synthetase comprises an amino acid recognition region in the catalytic domain (CD), and a mutation exists in said amino acid recognition region, wherein said amino acid recognition region specifically binds alanine in a wild-type alanyl-tRNA synthetase.

9. The noncanonical amino acid genetic encoding system according to claim 8,
wherein the catalytic domain of said chimeric alanyl-tRNA synthetase is derived from Archaeoglobus fulgidus (Af), and wherein said mutation is selected from a group consisting of: W191F/V214G/T243V, W191F/V214G, W191F/V214G/T243S, W191F/V214G/T243C, W191F/V214G/T243L, W191F/V214G/T243I, W191F/V214G/T243F, W191F/V214G/T243A, W191Y/V214G/T243V, W191Y/V214G, W191Y/V214G/T243S, W191Y/V214G/T243C, W191Y/V214G/T243L, W191Y/V214G/T243I, W191Y/V214G/T243F, W191Y/V214G/T243A, W191L/V214G/T243V, W191L/V214G, W191L/V214G/T243S, W191L/V214G/T243C, W191L/V214G/T243L, W191L/V214G/T243I, W191L/V214G/T243F, W191L/V214G/T243A, V214G/T243V, V214G, V214G/T243S, V214G/T243C, V214G/T243L, V214G/T243I, V214G/T243F, V214G/T243A, W191H/V214G/T243V, W191H/V214G, W191H/V214G/T243S, W191H/V214G/T243C, W191H/V214G/T243L, W191H/V214G/T243I, W191H/V214G/T243F, W191H/V214G/T243A, W191F/V214A/T243V, W191F/V214A, W191F/V214A/T243S, W191F/V214A/T243C, W191F/V214A/T243L, W191F/V214A/T243I, W191F/V214A/T243F, W191F/V214A/T243A, W191F/V214G/D242S/T243V, W191H/V214G/D242S/T243V, W191Y/V214G/D242S/T243V, W191F/T212S/V214G/T243V, W191F/T212A/V214G/T243V, W191F/T212S/V214G/, and W191H/T212S/V214G/T243V, wherein the mutation position numbering corresponds to the position in the wild-type alanyl-tRNA synthetase of Archaeoglobus fulgidus.

10. The noncanonical amino acid genetic encoding system according to claim 1,
wherein an efficiency-enhancing mutation further exists in the catalytic domain of the chimeric alanyl-tRNA synthetase, and said efficiency-enhancing mutation exists in a region corresponding to an A3-U70/G3-U70 binding domain of tRNA.

11. The noncanonical amino acid genetic encoding system according to claim 10,
wherein the catalytic domain of said chimeric alanyl-tRNA synthetase is derived from Archaeoglobus fulgidus (Af), and wherein the catalytic domain comprises one or more mutations selected from a group consisting of: N359K, N359A, and N359V,
wherein the mutation position numbering corresponds to the position in the wild-type alanyl-tRNA synthetase of Archaeoglobus fulgidus.

12. The noncanonical amino acid genetic encoding system according to claim 1,
wherein the sequence of the chimeric alanyl-tRNA synthetase is shown in any one of SEQ ID NOs: 56-75.

13. The noncanonical amino acid genetic encoding system according to claim 1,
wherein said system further comprises: (a) a host cell, wherein said host cell is a prokaryotic cell or a eukaryotic cell; and/or (b) a transcription template or a nucleic acid encoding the transcription template, wherein said transcription template comprises a codon encoding a non-natural amino acid, and said system further comprises a recombinant tRNA capable of recognizing said codon.

14. A method for preparing a polypeptide or protein comprising a noncanonical amino acid, comprising the step of: expressing said polypeptide or protein in a host cell or a cell-free system using the noncanonical amino acid genetic encoding system of claim 1.

15. The method according to claim 14, wherein said noncanonical amino acid is an Aib or aib like amino acid having the following structure: 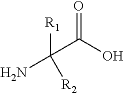
wherein R1 and/or R2 are H, C₁₋₅ alkyl, C₁₋₅—OH, or C₁₋₅—SH.

16. The method according to claim 14, wherein the polypeptide comprising the noncanonical amino acid is selected from the group consisting of: alpha-1 antitrypsin, angiostatin, antihemolytic factor, antibodies, apolipoproteins, atrial natriuretic peptide, C-X-C chemokines, Hedgehog proteins, hemoglobin, hepatocyte growth factor (HGF), hirudin, insulin, insulin-like growth factor (IGF), keratinocyte growth factor (KGF), lactoferrin, leukemia inhibitory factor, luciferase, Neurturin, neutrophil inhibitory factor (NIF), oncostatin M, osteogenic protein, parathyroid hormone, PD-ECSF, PDGF, pleiotrophin, protein A, protein G, pyrogenic exotoxin A, B or C, relaxin, renin, SCF, soluble complement receptor I, soluble interleukin receptor, soluble TNF receptor, somatomedin, streptokinase, superantigens, staphylococcal enterotoxins, superoxide dismutase (SOD), toxic shock syndrome toxin, thymosin alpha 1, tissue plasminogen activator, tumor growth factor (TGF), tumor necrosis factor and corticosterone, GAL4, erythropoietin (EPO), human growth hormone, T39765, NAP-2, ENA-78, Gro-a, Gro-b, Gro-c, IP-10, GCP-2, NAP-4, SDF-1, PF4, MIG, calcitonin, c-kit ligand, CC chemokines, monocyte chemoattractant protein-1, monocyte chemoattractant protein-2, monocyte chemoattractant protein-3, monocyte inflammatory protein-1alpha, monocyte inflammatory protein-1beta, RANTES, I309, R83915, R91733, HCC1, T58847, D31065, T64262, CD40, CD40 ligand, C-kit ligand, collagen, colony stimulating factor (CSF), complement factor 5a, complement inhibitors, complement receptor 1, DHFR, epithelial neutrophil activating peptide-78, GROalpha/MGSA, GRObeta, GROgamma, MIP-1alpha, MIP-1delta, MCP-1, epidermal growth factor (EGF), epithelial neutrophil activating peptide, exfoliative toxin, factor IX, factor VII, factor VIII, factor X, fibroblast growth factor (FGF), fibrinogen, fibronectin, G-CSF, GM-CSF, glucocerebrosidase, gonadotropin, human serum albumin, ICAM-1, ICAM-1 receptor, LFA-1, LFA-1 receptor, IGF-I, IGF-II, IFN-alpha, IFN-beta, IFN-gamma, IL-1, IL-2, IL-3, IL-4, IL-5, IL-6, IL-7, IL-8, IL-9, IL-10, IL-11, IL-12, SEA, SEB, SEC1, SEC2, SEC3, SED, SEE, soluble I-CAM1, TGF-alpha, TGF-beta, tumor necrosis factor alpha, tumor necrosis factor beta, tumor necrosis factor receptor (TNFR), VLA-4 protein, VCAM-1 protein, vascular endothelial growth factor (VEGF), urokinase, Mos, Ras, Raf, Met, p53, Tat, Fos, Myc, Jun, Myb, Rel, estrogen receptor, progesterone receptor, testosterone receptor, aldosterone receptor, LDL receptor, SCF/c-Kit, CD40L/CD40, VLA-4/VCAM-1, ICAM-1/LFA-1, and hyaluronan/CD44.

17. The method according to claim 14, wherein said polypeptide is a GLP-1 analog or a fusion protein containing a GLP-1 analog, and wherein the polypeptide comprises the sequence set forth in any one of SEQ ID NOs: 76-80, 112-117.

18. A method of preparing a polypeptide or protein containing one or more Aib or Aib like amino acids, the method comprising utilizing the system of claim 1.


SEQUENCE LISTING (155 sequences)

SEQ ID NO: 1 - protein, 149 aa, synthetic construct
  REGION 1..149: note = 1 Mb149
MDKKPLDVLISATGLWMSRTGTLHKIKHHEVSRSKIYIEMACGDHLVVNNSRSCRTARAFRHHKYRKTCKRCRVSDEDINNFLTRSTESKNSVKVRVVSAPKVKKAMPKSVSRAPKPLENSVSAKASTNTSRSVPSPAKSTPNSSVPAS

SEQ ID NO: 2 - protein, 149 aa, synthetic construct
  REGION 1..149: note = 2 Mm149
MDKKPLNTLISATGLWMSRTGTIHKIKHHEVSRSKIYIEMACGDHLVVNNSRSSRTARALRHHKYRKTCKRCRVSDEDLNKFLTKANEDQTSVKVKVVSAPTRTKKAMPKSVARAPKPLENTEAAQAQPSGSKFSPAIPVSTQESVSVP

SEQ ID NO: 3 - protein, 149 aa, synthetic construct
  REGION 1..149: note = 3ch149
MDKKPLDVLISATGLWMSRTGTLHKIKHYEISRSKIYIEMACGDHLVVNNSRSCRPARAFRYHKYRKTCKRCRVSDEDINNFLTRSTEGKTSVKVKVVSEPKVKKAMPKSVSRADKELENPVSAKASTDTSRSVPSPAKSTPNSPVPTS

SEQ ID NO: 4 - protein, 60 aa, synthetic construct
  REGION 1..60: note = 4 G1
MVVKFTDSQIQHLMEYGDNDWSEAEFEDAAARDKEFSSQFSKLKSANDKGLKDVIANPRN

SEQ ID NO: 5 - protein, 60 aa, synthetic construct
  REGION 1..60: note = 5Ma
MTVKYTDAQIQRLREYGNGTYEQKVFEDLASRDAAFSKEMSVASTDNEKKIKGMIANPSR

SEQ ID NO: 6 - protein, 60 aa, synthetic construct
  REGION 1..60: note = 6 lum
MDTRLTPAQAQRIREMGGTVDPSLAFSSEAERESAFQRISADLQGANLAKIRRCAEAPER

SEQ ID NO: 7 - protein, 60 aa, synthetic construct
  REGION 1..60: note = 7 deb
MNSSWTEVQRHRLKELNGAEKDLETAFGDDLQRNRAFQKLEKQLVYQERKRLDRLLDTRF

SEQ ID NO: 8 - protein, 60 aa, synthetic construct
  REGION 1..60: note = 8nitra
MASKIRFTRGQIHRLIELGAEPTELERDFETEAERDKEFNKIAENLARKNLKNIKDFLEQ

SEQ ID NO: 9 - protein, 60 aa, synthetic construct
  REGION 1..60: note = 9 1r26
MAEHFTDAQIQRLREYGNGTYKDMEFADVSAREKAFTKLMSDASRDNESALKGMIAHPAR

SEQ ID NO: 10 - protein, 148 aa, synthetic construct
  REGION 1..148: note = 10 mbur
MEKQLLDVLVELNGVWLSRSGLLHGIRNFEITTKHIHIETDCGARFTVRNSRSSRSARSLRHNKYRKPCKRCRPADEQIDRFVKKTFKEKRQTVSVFSSPKKHVPKKPKVAVIKSFSISTPSPKEASVSNSIPTPSISVVKDEVKVPE

SEQ ID NO: 11 - protein, 240 aa, synthetic construct
  REGION 1..240: note = 11 mm-ipye
MDKKPLNTLISATGLWMSRTGTIHKIKHHEVSRSKIYIEMACGDHLVVNNSRSSRTARALRHHKYRKTCKRCRVSDEDLNKFLTKANEDQTSVKVKVVSAPTRTKKAMPKSVARAPKPLENTEAAQAQPSGSKFSPAIPVSTQESVSVPASVSTSISSISTGATASALVKGNTNPITSMSAPVQASAPALTKSQTDRLEVLLNPKDEISLNSGKPFRELESELLSRRKKDLQQIYAEERE

SEQ ID NO: 12 - protein, 206 aa, synthetic construct
  REGION 1..206: note = 12 mb-ipye
MDKKPLDVLISATGLWMSRTGTLHKIKHYEISRSKIYIEMACGDHLVVNNSRSCRPARAFRYHKYRKTCKRCRVSDEDINNFLTRSTEGKTSVKVKVVSEPKVKKAMPKSVSRAPKPLENPVSAKASTDTSRSVPSPAKSTPNSPVPTSAPAPSLTRSQLDRVEALLSPEDKISLNMAKPFRELEPELVTRRKNDFQRLYTNDRED

SEQ ID NO: 13 - protein, 205 aa, synthetic construct
  REGION 1..205: note = 13 ch-IPYE
MDKKPLDVLISATGLWMSRTGTLHKIKHYEISRSKIYIEMACGDHLVVNNSRSCRPARAFRYHKYRKTCKRCRVSDEDINNFLTRSTEGKTSVKVKVVSEPKVKKAMPKSVSRAPKPLENPVSAKASTDTSRSVPSPAKSTPNSPVPTSASAPALTKSQTDRLEVLLNPKDEISLNSGKPFRELESELLSRRKKDLQQIYAEERE

SEQ ID NO: 14 - protein, 205 aa, synthetic construct
  REGION 1..205: note = 14 mm-comm1
MNKKPLNTLISATGLWMSRTGTIHKIKHHEISRSKIYIEMACGDHLVVNNSRSSRPARALKYHKYRKTCKRCRVSDEDLNKFLTKANEDQTSVKVKVVSAPTRTKKAMPKSVARAPKPLENSEAAQAQPSGSKFSPAIPVSTQESVSVPASAPALTKRQTDRLEVLLNPKDEISLNSGKPFRELESELLSRRKKDLQQIYAEERE

SEQ ID NO: 15 - protein, 205 aa, synthetic construct
  REGION 1..205: note = 15 mm-comm2
MNKKPLYTLISATGLWMSRTGTIHKIKHHEISRSKIYIEMACGDHLVVNNSRSSRPARALKYLKYRNTCKRCRWSDEDLNKFLTKANEDQTSVKVKVVSAPTRTKKAMPKSVARAPKPLENSEAAQAQPSGSKFSPAIPVSTQESVSVPASAPALTKSQTDRLEVLLNPKDEISLNSGKPFRELESELLSRRKKDLQQIYAEERE

SEQ ID NO: 16 - protein, 383 aa, synthetic construct
  REGION 1..383: note = 16 EcAlaRS-CD
MSKSTAEIRQAFLDFFHSKGHQVVASSSLVPHNDPTLLFTNAGMNQFKDVFLGLDKRNYSRATTSQRCVRAGGKHNDLENVGYTARHHTFFEMLGNFSFGDYFKHDAIQFAWELLTSEKWFALPKERLWVTVYESDDEAYEIWEKEVGIPRERIIRIGDNKGAPYASDNFWQMGDTGPCGPCTEIFYDHGDHIWGGPPGSPEEDGDRYIEIWNIVFMQFNRQADGTMEPLPKPSVDTGMGLERIAAVLQHVNSNYDIDLFRTLIQAVAKVTGATDLSNKSLRVIADHIRSCAFLIADGVMPSNENRGYVLRRIIRRAVRHGNMLGAKETFFYKLVGPLIDVMGSAGEDLKRQQAQVEQVLKTEEEQFARTLERGLALLDEELA

SEQ ID NO: 17 - protein, 485 aa, synthetic construct
  REGION 1..485: note = 17 AfAlaRS-CD
MTLDEEYLDITFLTENGFVRKRCPKCGKHFWTADPEREICGDPPCESYSFIGNPVFKKPFELDEMREYYLNFFERRGHGRIERYPVVARWRTDIYLTIASIADFQPFVTSGVAPPPANPLTISQPCIRLDDLDSVGRTGRHLTLFEMMAHHAFNYPGKEIYWKNETVAYCTELLNELGVKKEDIVYKEEPWAGGGNAGPCLEAIVGGLEVATLVFMNLEEHPEGDIEIKGARYRKMDNYIVDTGYGLERFVWASKGTPTVYDAIFPEVVDTIIDNSNVSFNREDERVRRIVAESSKLAGIMGELRGERLNQLRKSVADTVGVSVEELEGIVVPLEKVYSLADHTRCILFMLGDGLVPSNAGAGYLARLMIRRSLRLAEELELGLDLYDLVEMHKKILGFEFDVPLSTVQEILELEKERYRTTVSKGTRLVERLVERKKKLEKDDLIELYDSHGIPVELAVGIAAEKGAEVEMPKDIYAELAKRHS

SEQ ID NO: 18 - protein, 491 aa, synthetic construct
  REGION 1..491: note = 18 PhAlaRS-CD
MEFIMKTRMFEEEGWIRKKCKVCGKPFWTLDPDRETCGDPPCDEYQFIGKPGIPRKYTLDEMREKFLRFFEKHEIYPHGRVKRYPVLPRWRDDVLLVGASIMDFQPWVISGEADPPANPLVISQPSIRFTDIDNVGITGRHFTIFEMMAHHAFNYPGKPIYWMDETVELAFEFFTKELKMKPEDITFKENPWAGGGNAGPAFEVLYRGLEVATLVFMQYKKAPENAPQDQVVVIKGEKYIPMETKVVDTGYGLERLVWMSQGTPTAYDAVLGYVVEPLKKMAGIEKIDEKILMENSRLAGMFDIEDLGDLRYLREQVAKRVGITVEELEKAIRPYELIYAIADHTKALTFMLADGVVPSNVKAGYLARLLIRKSIRHLRELGLEVPLSEIVALHIKELHKTFPEFKEMEDIILEMIELEEKKYAETLRRGSDLVRREIAKLKKKGIKEIPVEKLVTFYESHGLTPEIVKEIAEKEGVKVNIPDNFYSMVAK

SEQ ID NO: 19 - protein, 464 aa, synthetic construct
  REGION 1..464: note = 19 HmAlaRS-CD
AWRGLSHRPLSSEPPAAKASAVRAAFLNFFRDRHGHRLVPSASVRPRGDPSLLFVNAGMNQFKPIFLGTVDPRSEMAGFRRVANSQKCVRAGGHHNDLEDVGRDLSHHTFFEMLGNWAFGGEYFKEEACNMAWELLTQVYGIPEERLWISYFDGDPKAGLDPDLETRDIWLSLGVPASRVLSFGPQENFWEMGDTGPCGPCTEIHYDLAGGVGAPQLVELWNLVFMQHNREADGSLQPLPQRHVDTGMGLERLVAVLQGKHSTYDTDLFSPLLNAIQQGCRAPPYLGRVGVADEGRTDTAYRVVADHIRTLSVCISDGIFPGMSGPPLVLRRILRRAVRFSMEILKAPPGFLGSLVPVVVETLGDAYPELQRNSAQIANLVSEDEAAFLASLERGRRIIDRTLRTLGPSDMFPAEVAWSLSLCGDLGLPLDMVELMLEEKGVQLDSAGLERLAQEEAQHRARQA

SEQ ID NO: 20 - protein, 438 aa, synthetic construct
  REGION 1..438: note = 20 HsAlaRS-CD
MDSTLTASEIRQRFIDFFKRNEHTYVHSSATIPLDDPTLLFANAGMNQFKPIFLNTIDPSHPMAKLSRAANTQKCIRAGGKHNDLDDVGKDVYHHTFFEMLGSWSFGDYFKELACKMALELLTQEFGIPIERLYVTYFGGDEAAGLEADLECKQIWQNLGLDDTKILPGNMKDNFWEMGDTGPCGPCSEIHYDRIGGRDAAHLVNQDDPNVLEIWNLVFIQYNREADGILKPLPKKSIDTGMGLERLVSVLQNKMSNYDTDLFVPYFEAIQKGTGARPYTGKVGAEDADGIDMAYRVLADHARTITVALADGGRPDNTGRGYVLRRILRRAVRYAHEKLNASRGFFATLVDVVVQSLGDAFPELKKDPDMVKDIINEEEVQFLKTLSRGRRILDRKIQSLGDSKTIPGDTAWLLYDTYGFPVDLTGLIAEEKGLVVDM

SEQ ID NO: 21 - protein, 491 aa, synthetic construct
  REGION 1..491: note = 21 PfAlaRS-CD
MEFVMKTRMFEEEGWIRKTCKVCGKPFWTLDPDRETCGDPPCDEYQFIGKPGIPKKYTLDEMREKFLSFFEKHEVYPHGRVKRYPVLPRWRDDVLLVGASIMDFQPWVISGEADPPANPLTISQPSIRFTDIDNVGITGRHFTIFEMMAHHAFNYPGKHIYWIDETVELAFEFFTKELKMKPEDITFKENPWAGGGNAGPAFEVLYKGLEVATLVFMQYKKAPANADPSQVVIIKGEKYVPMETKVVDTGYGLERLVWMSQGTPTAYDAVLGYVIEPLKRMAGVEKIDERILMENSRLAGMFDIEDMGDLRYLREQVAKRVGISVEELERLIRPYELIYAIADHTKALTFMLADGVVPSNVKAGYLARLLIRKSIRHLRELGLEVPLAEIVAMHIKELSPTFPEFKEMEDIILEMIELEEKKYAETLRRGSDLVKREIAKLKKKGANEIPLEKLITFYESHGLTPEIVKEIAEKEGVKVHIPDNFYSLVAK

SEQ ID NO: 22 - protein, 489 aa, synthetic construct
  REGION 1..489: note = 22 MpAlaRS-CD
MLEDEYQLDFFSENGFVRKQCSKCGKFYWTRDLDRNTCGDAPCDPYSFIGNPVFNKKFDLAEMREYYLKFFEDQGHTRLERYPVVARWRDDIYLTIASIADFQPFVTSGQVRPPANPLTISQPCIRLSDLDAVGRSGRHLTTFEMMAHHAFNKKGEEIYWKDRTMELCDELFNSLGVDPLAVTYKEEPWAGGGNAGPCVEALIGGLEVATLVFMDLEQSKDGTIPIKGENYRKMDNYIVDTGYGLERFVWASKGSPTIYDAVFPTIVNELMQLAGLEHQIDDPEYANILSQSARLAGLMDVSEKANLLQLRKQVASSIGTSVEKLSSIMEPVENIYAITDHTRCLTFMLADGIIPSNVKAGYLARLVLRRTLRMMKDMDINTSLSDIVRMHIKNLPEYPEFSEKFDVIEDILAHEEMKFDETLERGRRMIQKSAKHYKSTGEKIPLPTLIEMYDSHGIPPEISKEAASEMGVEVDLPDNFYSLVAEGHS

SEQ ID NO: 23 - protein, 472 aa, synthetic construct
  REGION 1..472: note = 23 TtAlaRS-CD
MLSTKLFETAGFHRKQCPMCKSYFWTLRQDQVYCGDQPCVPYGFIGNSPVRVPVESIRDLRERFLSFFEKRGHARIKRYPVVARWREDVFLVGASIYDFQPWVTSGAVPPPANPLTISQPSIRLTDVDKVGRSGRHLTGFEMMAHHAFNYPDKHIYWISETTEYAYEFFTKELGIPEDEITFKESVWEGGGNAGESFEVLVRGLEVATLVFMHYEVKDGKYVELPLKIVDTGYGLERIYWMAKGSPTVYDAVFGPFLSAARKKLGVPEPPAHILGKASVYFGQMDPEVIGLAKAYDLVAEKIGIDPREFREIMRPQEALYVLADHSRTVSWMIADGVIPSNTGAGYLARLLIRRALKSLWIAGIETSLVDLFDLHLGFLRDDYPEVWQSRDIILELIDLEEKKYKDVIKSAPAAVRRALEKAGGKSLKAEQLVELYDSYGIPPEVAQEVARKLGAEAEVPDDFYSRLAARHQ

SEQ ID NO: 24 - protein, 488 aa, synthetic construct
  REGION 1..488: note = 24 TsAlaRS-CD
MSMDMTTRMFKEEGWIRKQCPKCGKFFWTLDPDRETCGDPPCDEYSFIGKPGIPKKYTLEEMREAFLRFFEKHDHGRVKRFPVLPRWRDDVLLVGASIMDFQPWVISGEADPPANPLTISQPSIRFTDIDNVGITGRHFTIFEMMAHHAFNYPGKPIYWMDETVELAFEFFTKELGMKAEDITFKENPWAGGGNAGPAFEVLYRGLEVATLVFMQYKKAPENADPSQVVEIKGDYYVPMETKVVDTGYGLERLVWMSHGTPTAYDAVLGYVVEPLKRMAGIERIDERILMENSRLAGMFDIEDMGDLRYLRGQVAKRVGISVEELEKAVRPYELIYAIADHTKALTFMLADGVIPSNVKAGYLARLLIRKSIRHLRELGLEVPLAEIVAMHIKELSPTFPEFKEMEDVILDIINVEEKRYQETLKRGSDLVKREIARLKKAGKNEMPLEKLILFYESHGLTPEIVREVAEKEGVKVEIPDNFYTLVAK

SEQ ID NO: 25 - protein, 420 aa, synthetic construct
  REGION 1..420: note = 25 PaAlaRS-CD
MSQPFRSADIRQAFIDFFISKQHTPVASSSLIPHNDPTLLFTNAGMNQFKETFLGMEPRDYTRAVTSQKCVRAGGKHNDLDNVGYTARHHTFFEMLGNFSFGDYFKQAGIGYIWEFLTSDEWLAIDKNRLYVTIYETDDEAFDIWHKDIGIPSERIIRIGDNKGAPYASDNFWTMGDTGPCGPCTEVFYDHGADIEGGLPGTPEEDGDRYIEIWNCVFMQFNRQKDGTMLPLPAPSVDTGMGLERISAIMQGVHGNYEIDLFVHLMDAAAEILEIENQQQSSLKVIADHIRAVSFLIADGVTPSNEGRGYVLRRVIRRAVRHGNKLGADSDFFYKMVAPLVAEMGIAYPELKDKQSVIENTIQKEEAQFAKTLAQGLRLLASELEGLQDGDTLSGEAAFKLYDTYGFPVDLTADITRERG

SEQ ID NO: 26 - protein, 556 aa, synthetic construct
  REGION 1..556: note = 26chAlaRS-1
MSKSTAEIRQAFLDFFHSKGHQVVASSSLVPHNDPTLLFTNAGMNQFKDVFLGLDKRNYSRATTSQRCVRAGGKHNDLENVGYTARHHTFFEMLGNFSFGDYFKHDAIQFAWELLTSEKWFALPKERLWVTVYESDDEAYEIWEKEVGIPRERIIRIGDNKGAPYASDNFWQMGDTGPCGPCTEIFYDHGDHIWGGPPGSPEEDGDRYIEIWNIVFMQFNRQADGTMEPLPKPSVDTGMGLERIAAVLQHVNSNYDIDLFRTLIQAVAKVTGATDLSNKSLRVIADHIRSCAFLIADGVMPSNENRGYVLRRIIRRAVRHGNMLGAKETFFYKLVGPLIDVMGSAGEDLKRQQAQVEQVLKTEEEQFARTLERGLALLDEELAGGGSGGGSGGGSGGGSGGGSGGGSMDKKPLDVLISATGLWMSRTGTLHKIKHYEISRSKIYIEMACGDHLVVNNSRSCRPARAFRYHKYRKTCKRCRVSDEDINNFLTRSTEGKTSVKVKVVSEPKVKKAMPKSVSRAPKPLENPVSAKASTDTSRSVPSPAKSTPNSPVPTS

SEQ ID NO: 27 - protein, 696 aa, synthetic construct
  REGION 1..696: note = 27 chAlaRS-2
MDKKPLDVLISATGLWMSRTGTLHKIKHYEISRSKIYIEMACGDHLVVNNSRSCRPARAFRYHKYRKTCKRCRVSDEDINNFLTRSTEGKTSVKVKVVSEPKVKKAMPKSVSRAPKPLENPVSAKASTDTSRSVPSPAKSTPNSPVPTSASAPALTKSQTDRLEVLLNPKDEISLNSGKPFRELESELLSRRKKDLQQIYAEEREGGGSGGMTLDEEYLDITFLTENGFVRKRCPKCGKHFWTADPEREICGDPPCESYSFIGNPVFKKPFELDEMREYYLNFFERRGHGRIERYPVVARWRTDIYLTIASIADFQPFVTSGVAPPPANPLTISQPCIRLDDLDSVGRTGRHLTLFEMMAHHAFNYPGKEIYWKNETVAYCTELLNELGVKKEDIVYKEEPWAGGGNAGPCLEAIVGGLEVATLVFMNLEEHPEGDIEIKGARYRKMDNYIVDTGYGLERFVWASKGTPTVYDAIFPEVVDTIIDNSNVSFNREDERVRRIVAESSKLAGIMGELRGERLNQLRKSVADTVGVSVEELEGIVVPLEKVYSLADHTRCILFMLGDGLVPSNAGAGYLARLMIRRSLRLAEELELGLDLYDLVEMHKKILGFEFDVPLSTVQEILELEKERYRTTVSKGTRLVERLVERKKKLEKDDLIELYDSHGIPVELAVGIAAEKGAEVEMPKDIYAELAKRHS

SEQ ID NO: 28 - protein, 640 aa, synthetic construct
  REGION 1..640: note = 28 chAlaRS-3
MDKKPLDVLISATGLWMSRTGTLHKIKHYEISRSKIYIEMACGDHLVVNNSRSCRPARAFRYHKYRKTCKRCRVSDEDINNFLTRSTEGKTSVKVKVVSEPKVKKAMPKSVSRAPKPLENPVSAKASTDTSRSVPSPAKSTPNSPVPTSGGGSGGMTLDEEYLDITFLTENGFVRKRCPKCGKHFWTADPEREICGDPPCESYSFIGNPVFKKPFELDEMREYYLNFFERRGHGRIERYPVVARWRTDIYLTIASIADFQPFVTSGVAPPPANPLTISQPCIRLDDLDSVGRTGRHLTLFEMMAHHAFNYPGKEIYWKNETVAYCTELLNELGVKKEDIVYKEEPWAGGGNAGPCLEAIVGGLEVATLVFMNLEEHPEGDIEIKGARYRKMDNYIVDTGYGLERFVWASKGTPTVYDAIFPEVVDTIIDNSNVSFNREDERVRRIVAESSKLAGIMGELRGERLNQLRKSVADTVGVSVEELEGIVVPLEKVYSLADHTRCILFMLGDGLVPSNAGAGYLARLMIRRSLRLAEELELGLDLYDLVEMHKKILGFEFDVPLSTVQEILELEKERYRTTVSKGTRLVERLVERKKKLEKDDLIELYDSHGIPVELAVGIAAEKGAEVEMPKDIYAELAKRHS

SEQ ID NO: 29 - protein, 594 aa, synthetic construct
  REGION 1..594: note = 29 chAlaRS-4
MDKKPLDVLISATGLWMSRTGTLHKIKHYEISRSKIYIEMACGDHLVVNNSRSCRPARAFRYHKYRKTCKRCRVSDEDINNFLTRSTEGKTSVKVKVVSEPKVGGGSGGMTLDEEYLDITFLTENGFVRKRCPKCGKHFWTADPEREICGDPPCESYSFIGNPVFKKPFELDEMREYYLNFFERRGHGRIERYPVVARWRTDIYLTIASIADFQPFVTSGVAPPPANPLTISQPCIRLDDLDSVGRTGRHLTLFEMMAHHAFNYPGKEIYWKNETVAYCTELLNELGVKKEDIVYKEEPWAGGGNAGPCLEAIVGGLEVATLVFMNLEEHPEGDIEIKGARYRKMDNYIVDTGYGLERFVWASKGTPTVYDAIFPEVVDTIIDNSNVSFNREDERVRRIVAESSKLAGIMGELRGERLNQLRKSVADTVGVSVEELEGIVVPLEKVYSLADHTRCILFMLGDGLVPSNAGAGYLARLMIRRSLRLAEELELGLDLYDLVEMHKKILGFEFDVPLSTVQEILELEKERYRTTVSKGTRLVERLVERKKKLEKDDLIELYDSHGIPVELAVGIAAEKGAEVEMPKDIYAELAKRHS

SEQ ID NO: 30 - protein, 657 aa, synthetic construct
  REGION 1..657: note = 30 chAlaRS-5
MDKKPLDVLISATGLWMSRTGTLHKIKHYEISRSKIYIEMACGDHLVVNNSRSCRPARAFRYHKYRKTCKRCRVSDEDINNFLTRSTEGKTSVKVKVVSEPKVKKAMPKSVSRAPKPLENPVSAKASTDTSRSVPSPAKSTPNSPVPTSASAPALTKSQTDRLEVLLNPKDEISLNSGKPFRELESELLSRRKKDLQQIYAEEREGGGSGGAWRGLSHRPLSSEPPAAKASAVRAAFLNFFRDRHGHRLVPSASVRPRGDPSLLFVNAGMNQFKPIFLGTVDPRSEMAGFRRVANSQKCVRAGGHHNDLEDVGRDLSHHTFFEMLGNWAFGGEYFKEEACNMAWELLTQVYGIPEERLWISYFDGDPKAGLDPDLETRDIWLSLGVPASRVLSFGPQENFWEMGDTGPCGPCTEIHYDLAGGVGAPQLVELWNLVFMQHNREADGSLQPLPQRHVDTGMGLERLVAVLQGKHSTYDTDLFSPLLNAIQQGCRAPPYLGRVGVADEGRTDTAYRVVADHIRTLSVCISDGIFPGMSGPPLVLRRILRRAVRFSMEILKAPPGFLGSLVPVVVETLGDAYPELQRNSAQIANLVSEDEAAFLASLERGRRIIDRTLRTLGPSDMFPAEVAWSLSLCGDLGLPLDMVELMLEEKGVQLDS

SEQ ID NO: 31 - protein, 557 aa, synthetic construct
  REGION 1..557: note = 31 chAlaRS-6
MVVKFTDSQIQHLMEYGDNDWSEAEFEDAAARDKEFSSQFSKLKSANDKGLKDVIANPRNGGGSGGGGGSGGMTLDEEYLDITFLTENGFVRKRCPKCGKHFWTADPEREICGDPPCESYSFIGNPVFKKPFELDEMREYYLNFFERRGHGRIERYPVVARWRTDIYLTIASIADFQPFVTSGVAPPPANPLTISQPCIRLDDLDSVGRTGRHLTLFEMMAHHAFNYPGKEIYWKNETVAYCTELLNELGVKKEDIVYKEEPWAGGGNAGPCLEAIVGGLEVATLVFMNLEEHPEGDIEIKGARYRKMDNYIVDTGYGLERFVWASKGTPTVYDAIFPEVVDTIIDNSNVSFNREDERVRRIVAESSKLAGIMGELRGERLNQLRKSVADTVGVSVEELEGIVVPLEKVYSLADHTRCILFMLGDGLVPSNAGAGYLARLMIRRSLRLAEELELGLDLYDLVEMHKKILGFEFDVPLSTVQEILELEKERYRTTVSKGTRLVERLVERKKKLEKDDLIELYDSHGIPVELAVGIAAEKGAEVEMPKDIYAELAKRHS

SEQ ID NO: 32 - protein, 699 aa, synthetic construct
  REGION 1..699: note = 32 chAlaRS-7
MDKKPLDVLISATGLWMSRTGTLHKIKHYEISRSKIYIEMACGDHLVVNNSRSCRPARAFRYHKYRKTCKRCRVSDEDINNFLTRSTEGKTSVKVKVVSEPKVKKAMPKSVSRAPKPLENPVSAKASTDTSRSVPSPAKSTPNSPVPTSASAPALTKSQTDRLEVLLNPKDEISLNSGKPFRELESELLSRRKKDLQQIYAEEREGGGSGGLEDEYQLDFFSENGFVRKQCSKCGKFYWTRDLDRNTCGDAPCDPYSFIGNPVFNKKFDLAEMREYYLKFFEDQGHTRLERYPVVARWRDDIYLTIASIADFQPFVTSGQVRPPANPLTISQPCIRLSDLDAVGRSGRHLTTFEMMAHHAFNKKGEEIYWKDRTMELCDELFNSLGVDPLAVTYKEEPWAGGGNAGPCVEALIGGLEVATLVFMDLEQSKDGTIPIKGENYRKMDNYIVDTGYGLERFVWASKGSPTIYDAVFPTIVNELMQLAGLEHQIDDPEYANILSQSARLAGLMDVSEKANLLQLRKQVASSIGTSVEKLSSIMEPVENIYAITDHTRCLTFMLADGIIPSNVKAGYLARLVLRRTLRMMKDMDINTSLSDIVRMHIKNLPEYPEFSEKFDVIEDILAHEEMKFDETLERGRRMIQKSAKHYKSTGEKIPLPTLIEMYASHGIPPEISKEAASEMGVEVDLPDNFYSLVAEGHS

SEQ ID NO: 33 - protein, 682 aa, synthetic construct
  REGION 1..682: note = 33 chAlaRS-8
MDKKPLDVLISATGLWMSRTGTLHKIKHYEISRSKIYIEMACGDHLVVNNSRSCRPARAFRYHKYRKTCKRCRVSDEDINNFLTRSTEGKTSVKVKVVSEPKVKKAMPKSVSRAPKPLENPVSAKASTDTSRSVPSPAKSTPNSPVPTSASAPALTKSQTDRLEVLLNPKDEISLNSGKPFRELESELLSRRKKDLQQIYAEEREGGGSGGLSTKLFETAGFHRKQCPMCKSYFWTLRQDQVYCGDQPCVPYGFIGNSPVRVPVESIRDLRERFLSFFEKRGHARIKRYPVVARWREDVFLVGASIYDFQPWVTSGAVPPPANPLTISQPSIRLTDVDKVGRSGRHLTGFEMMAHHAFNYPDKHIYWISETTEYAYEFFTKELGIPEDEITFKESVWEGGGNAGESFEVLVRGLEVATLVFMHYEVKDGKYVELPLKIVDTGYGLERIYWMAKGSPTVYDAVFGPFLSAARKKLGVPEPPAHILGKASVYFGQMDPEVIGLAKAYDLVAEKIGIDPREFREIMRPQEALYVLADHSRTVSWMIADGVIPSNTGAGYLARLLIRRALKSLWIAGIETSLVDLFDLHLGFLRDDYPEVWQSRDIILELIDLEEKKYKDVIKSAPAAVRRALEKAGGKSLKAEQLVELYASYGIPPEVAQEVARKLGAEAEVPDDFYSRLAARHQ

SEQ ID NO: 34 - protein, 697 aa, synthetic construct
  REGION 1..697: note = 34 chAlaRS-9
MDKKPLDVLISATGLWMSRTGTLHKIKHYEISRSKIYIEMACGDHLVVNNSRSCRPARAFRYHKYRKTCKRCRVSDEDINNFLTRSTEGKTSVKVKVVSEPKVKKAMPKSVSRAPKPLENPVSAKASTDTSRSVPSPAKSTPNSPVPTSASAPALTKSQTDRLEVLLNPKDEISLNSGKPFRELESELLSRRKKDLQQIYAEEREGGGSGGMDMTTRMFKEEGWIRKQCPKCGKFFWTLDPDRETCGDPPCDEYSFIGKPGIPKKYTLEEMREAFLRFFEKHDHGRVKRFPVLPRWRDDVLLVGASIMDFQPWVISGEADPPANPLTISQPSIRFTDIDNVGITGRHFTIFEMMAHHAFNYPGKPIYWMDETVELAFEFFTKELGMKAEDITFKENPWAGGGNAGPAFEVLYRGLEVATLVFMQYKKAPENADPSQVVEIKGDYYVPMETKVVDTGYGLERLVWMSHGTPTAYDAVLGYVVEPLKRMAGIERIDERILMENSRLAGMFDIEDMGDLRYLRGQVAKRVGISVEELEKAVRPYELIYAIADHTKALTFMLADGVIPSNVKAGYLARLLIRKSIRHLRELGLEVPLAEIVAMHIKELSPTFPEFKEMEDVILDIINVEEKRYQETLKRGSDLVKREIARLKKAGKNEMPLEKLILFYASHGLTPEIVREVAEKEGVKVEIPDNFYTLVAK

SEQ ID NO: 35 - protein, 703 aa, synthetic construct
  REGION 1..703: note = 35 chAlaRS-10
MDKKPLDVLISATGLWMSRTGTLHKIKHYEISRSKIYIEMACGDHLVVNNSRSCRPARAFRYHKYRKTCKRCRVSDEDINNFLTRSTEGKTSVKVKVVSEPKVKKAMPKSVSRAPKPLENPVSAKASTDTSRSVPSPAKSTPNSPVPTSASAPALTKSQTDRLEVLLNPKDEISLNSGKPFRELESELLSRRKKDLQQIYAEEREGGGSGGMTLDEEYLDITFLTENGFVRKRCPKCGKHFWTADPEREICGDPPCESYSFIGNPVVSQPFRSADIRQAFIDFFISKQHTPVASSSLIPHNDPTLLFTNAGMNQFKETFLGMEPRDYTRAVTSQKCVRAGGKHNDLDNVGYTARHHTFFEMLGNFSFGDYFKQAGIGYIWEFLTSDEWLAIDKNRLYVTIYETDDEAFDIWHKDIGIPSERIIRIGDNKGAPYASDNFWTMGDTGPCGPCTEVFYDHGADIEGGLPGTPEEDGDRYIEIWNCVFMQFNRQKDGTMLPLPAPSVDTGMGLERISAIMQGVHGNYEIDLFVHLMDAAAEILEIENQQQSSLKVIADHIRAVSFLIADGVTPSNEGRGYVLRRVIRRAVRHGNKLGADSDFFYKMVAPLVAEMGIAYPELKDKQSVIENTIQKEEAQFAKTLAQGLRLLASELEGLQDGDTLSGEAAFKLYDTYGFPVDLTADITRERGIVIDEAEFDEHMQAQRE

SEQ ID NO: 36 - RNA, 72 nt, synthetic construct
  misc_feature 1..72: note = 36 chAlaT-1
  variation 31: note = n = a, c, g or t
  variation 32: note = n = a, c, g or t
  variation 33: note = n = a, c, g or t
  source 1..72: mol_type = other RNA
ggagctatgatcatgtagatcgaacggactnnnaatccgttcagccgggttagattcccggtagctccacca

SEQ ID NO: 37 - RNA, 72 nt, synthetic construct
  misc_feature 1..72: note = 37 chAlaT-1_tcg
  source 1..72: mol_type = other RNA
ggagctatgatcatgtagatcgaacggactcgaaatccgttcagccgggttagattcccggtagctccacca

SEQ ID NO: 38 - RNA, 72 nt, synthetic construct
  misc_feature 1..72: note = 38 chAlaT-2
  variation 31: note = n = a, c, g or t
  variation 32: note = n = a, c, g or t
  variation 33: note = n = a, c, g or t
  source 1..72: mol_type = other RNA
ggactcgtgatcatgtagatcgaacggactnnnaatccgttcagccgggttagattcccggcgagtccacca

SEQ ID NO: 39 - RNA, 72 nt, synthetic construct
  misc_feature 1..72: note = 39 chAlaT-2_TCG
  source 1..72: mol_type = other RNA
ggactcgtgatcatgtagatcgaacggactcgaaatccgttcagccgggttagattcccggcgagtccacca

SEQ ID NO: 40 - RNA, 71 nt, synthetic construct
  misc_feature 1..71: note = 40 chAlaT-3
  variation 30: note = n = a, c, g or t
  variation 31: note = n = a, c, g or t
  variation 32: note = n = a, c, g or t
  source 1..71: mol_type = other RNA
ggactcggctccggcgagcaaacgggtctnnnaaacctgtaagcggggttcgaccccccgcgagtccacca

SEQ ID NO: 41 - RNA, 71 nt, synthetic construct
  misc_feature 1..71: note = 41 chAlaT-3_TCG
  source 1..71: mol_type = other RNA
ggactcggctccggcgagcaaacgggtctcgaaaacctgtaagcggggttcgaccccccgcgagtccacca

SEQ ID NO: 42 - RNA, 72 nt, synthetic construct
  misc_feature 1..72: note = 42 chAlaT-4
  variation 31: note = n = a, c, g or t
  variation 32: note = n = a, c, g or t
  variation 33: note = n = a, c, g or t
  source 1..72: mol_type = other RNA
ggactcgtgatcatgtagatcgaacggactnnnaatcctttcagccgggttagattcccggcgagtccacca

SEQ ID NO: 43 - RNA, 72 nt, synthetic construct
  misc_feature 1..72: note = 43 chAlaT-4_TCG
  source 1..72: mol_type = other RNA
ggactcgtgatcatgtagatcgaacggactcgaaatcctttcagccgggttagattcccggcgagtccacca

SEQ ID NO: 44 - RNA, 71 nt, synthetic construct
  misc_feature 1..71: note = 44 chAlaT-5
  variation 31: note = n = a, c, g or t
  variation 32: note = n = a, c, g or t
  variation 33: note = n = a, c, g or t
  source 1..71: mol_type = other RNA
ggactcgtggccaaggtggccgaacggactnnnaatccgttcaggggggttcgattcccccgagtccacca

SEQ ID NO: 45 - RNA, 71 nt, synthetic construct
  misc_feature 1..71: note = 45 chAlaT-5_TCG
  source 1..71: mol_type = other RNA
ggactcgtggccaaggtggccgaacggactcgaaatccgttcaggggggttcgattcccccgagtccacca

SEQ ID NO: 46 - RNA, 72 nt, synthetic construct
  misc_feature 1..72: note = 46 chAlaT-6
  variation 31: note = n = a, c, g or t
  variation 32: note = n = a, c, g or t
  variation 33: note = n = a, c, g or t
  source 1..72: mol_type = other RNA
ggactcgtggccaaggtggccgtgttgactnnnaatcaacacaggggggttcgattccccccgagtccacca

SEQ ID NO: 47 - RNA, 72 nt, synthetic construct
  misc_feature 1..72: note = 47 chAlaT-6_TCG
  source 1..72: mol_type = other RNA
ggactcgtggccaaggtggccgtgttgactcgaaatcaacacaggggggttcgattccccccgagtccacca

SEQ ID NO: 48 - RNA, 73 nt, synthetic construct
  misc_feature 1..73: note = 48 chAlaT-7
  variation 31: note = n = a, c, g or t
  variation 32: note = n = a, c, g or t
  variation 33: note = n = a, c, g or t
  source 1..73: mol_type = other RNA
ggactcgtggccaaggtggccagcgggtctnnnaaacctagccagacgggttcgattcctgtcgagtccacca

SEQ ID NO: 49 - RNA, 73 nt, synthetic construct
  misc_feature 1..73: note = 49 chAlaT-7_TCG
  source 1..73: mol_type = other RNA
ggactcgtggccaaggtggccagcgggtctcgaaaacctagccagacgggttcgattcctgtcgagtccacca

SEQ ID NO: 50 - protein, 6 aa, synthetic construct
  REGION 1..6: note = 50 linker1
GGGSGG

SEQ ID NO: 51 - protein, 24 aa, synthetic construct
  REGION 1..24: note = 51 linker 2
GGGSGGGSGGGSGGGSGGGSGGGS

SEQ ID NO: 52 - protein, 18 aa, synthetic construct
  REGION 1..18: note = 52 linker 3
APAPAPAPAPAPAPAPAP

SEQ ID NO: 53 - protein, 17 aa, synthetic construct
  REGION 1..17: note = 53 linker 4
AEAAAKEAAAKEAAAKA

SEQ ID NO: 54 - protein, 12 aa, synthetic construct
  REGION 1..12: note = 54 linker 5
GGGSGGGSGGGS

SEQ ID NO: 55 - protein, 18 aa, synthetic construct
  REGION 1..18: note = 55 linker 6
GGGSGGGSGGGSGGGSGG

SEQ ID NO: 56 - protein, 556 aa, synthetic construct
  REGION 1..556: note = 56 AibRS-1
MSKSTAEIRQAFLDFFHSKGHQVVASSSLVPHNDPTLLFTNAGMNQFKDVFLGLDKRNYSRATTSQRCVRAGGKHNDLENVGYTARHHTFFEMLGNFSFGDYFKHDAIQFAWELLTSEKWFALPKERLWVTVYESDDEAYEIWEKEVGIPRERIIRIGDNKGAPYASDNFHQMGDTGPCGPCTEIFYDHGDHIWGGPPGSPEEDGDRYIEIWNIGFMQFNRQADGTMEPLPKPSVDVGMGLERIAAVLQHVNSNYDIDLFRTLIQAVAKVTGATDLSNKSLRVIADHIRSCAFLIADGVMPSNENRGYVLRRIIRRAVRHGNMLGAKETFFYKLVGPLIDVMGSAGEDLKRQQAQVEQVLKTEEEQFARTLERGLALLDEELAGGGSGGGSGGGSGGGSGGGSGGGSMDKKPLDVLISATGLWMSRTGTLHKIKHYEISRSKIYIEMACGDHLVVNNSRSCRPARAFRYHKYRKTCKRCRVSDEDINNFLTRSTEGKTSVKVKVVSEPKVKKAMPKSVSRAPKPLENPVSAKASTDTSRSVPSPAKSTPNSPVPTS

SEQ ID NO: 57 - protein, 696 aa, synthetic construct
  REGION 1..696: note = 57 AibRS-2
MDKKPLDVLISATGLWMSRTGTLHKIKHYEISRSKIYIEMACGDHLVVNNSRSCRPARAFRYHKYRKTCKRCRVSDEDINNFLTRSTEGKTSVKVKVVSEPKVKKAMPKSVSRAPKPLENPVSAKASTDTSRSVPSPAKSTPNSPVPTSASAPALTKSQTDRLEVLLNPKDEISLNSGKPFRELESELLSRRKKDLQQIYAEEREGGGSGGMTLDEEYLDITFLTENGFVRKRCPKCGKHFWTADPEREICGDPPCESYSFIGNPVFKKPFELDEMREYYLNFFERRGHGRIERYPVVARWRTDIYLTIASIADFQPFVTSGVAPPPANPLTISQPCIRLDDLDSVGRTGRHLTLFEMMAHHAFNYPGKEIYWKNETVAYCTELLNELGVKKEDIVYKEEPHAGGGNAGPCLEAIVGGLEVATLGFMNLEEHPEGDIEIKGARYRKMDNYIVDVGYGLERFVWASKGTPTVYDAIFPEVVDTIIDNSNVSFNREDERVRRIVAESSKLAGIMGELRGERLNQLRKSVADTVGVSVEELEGIVVPLEKVYSLADHTRCILFMLGDGLVPSNAGAGYLARLMIRRSLRLAEELELGLDLYDLVEMHKKILGFEFDVPLSTVQEILELEKERYRTTVSKGTRLVERLVERKKKLEKDDLIELYASHGIPVELAVGIAAEKGAEVEMPKDIYAELAKRHS

SEQ ID NO: 58 - protein, 551 aa, synthetic construct
  REGION 1..551: note = 58 AibRS-3
MVVKFTDSQIQHLMEYGDNDWSEAEFEDAAARDKEFSSQFSKLKSANDKGLKDVIANPRNGGGSGGMTLDEEYLDITFLTENGFVRKRCPKCGKHFWTADPEREICGDPPCESYSFIGNPVFKKPFELDEMREYYLNFFERRGHGRIERYPVVARWRTDIYLTIASIADFQPFVTSGVAPPPANPLTISQPCIRLDDLDSVGRTGRHLTLFEMMAHHAFNYPGKEIYWKNETVAYCTELLNELGVKKEDIVYKEEPHAGGGNAGPCLEAIVGGLEVATLGFMNLEEHPEGDIEIKGARYRKMDNYIVDVGYGLERFVWASKGTPTVYDAIFPEVVDTIIDNSNVSFNREDERVRRIVAESSKLAGIMGELRGERLNQLRKSVADTVGVSVEELEGIVVPLEKVYSLADHTRCILFMLGDGLVPSAAGAGYLARLMIRRSLRLAEELELGLDLYDLVEMHKKILGFEFDVPLSTVQEILELEKERYRTTVSKGTRLVERLVERKKKLEKDDLIELYASHGIPVALAVGIAAEKGAEVEMPKDIYAELAKRHS

SEQ ID NO: 59 - protein, 696 aa, synthetic construct
  REGION 1..696: note = 59 AibRS-4
MDKKPLDVLISATGLWMSRTGTLHKIKHYEISRSKIYIEMACGDHLVVNNSRSCRPARAFRYHKYRKTCKRCRVSDEDINNFLTRSTEGKTSVKVKVVSEPKVKKAMPKSVSRAPKPLENPVSAKASTDTSRSVPSPAKSTPNSPVPTSASAPALTKSQTDRLEVLLNPKDEISLNSGKPFRELESELLSRRKKDLQQIYAEEREGGGSGGMTLDEEYLDITFLTENGFVRKRCPKCGKHFWTADPEREICGDPPCESYSFIGNPVFKKPFELDEMREYYLNFFERRGHGRIERYPVVARWRTDIYLTIASIADFQPFVTSGVAPPPANPLTISQPCIRLDDLDSVGRTGRHLTLFEMMAHHAFNYPGKEIYWKNETVAYCTELLNELGVKKEDIVYKEEPHAGGGNAGPCLEAIVGGLEVATLGFMNLEEHPEGDIEIKGARYRKMDNYIVDVGYGLERFVWASKGTPTVYDAIFPEVVDTIIDNSNVSFNREDERVRRIVAESSKLAGIMGELRGERLNQLRKSVADTVGVSVEELEGIVVPLEKVYSLADHTRCILFMLGDGLVPSAAGAGYLARLMIRRSLRLAEELELGLDLYDLVEMHKKILGFEFDVPLSTVQEILELEKERYRTTVSKGTRLVERLVERKKKLEKDDLIELYASHGIPVALAVGIAAEKGAEVEMPKDIYAELAKRHS

SEQ ID NO: 60 - protein, 696 aa, synthetic construct
  REGION 1..696: note = 60 AibRS-5
MDKKPLDVLISATGLWMSRTGTLHKIKHYEISRSKIYIEMACGDHLVVNNSRSCRPARAFRYHKYRKTCKRCRVSDEDINNFLTRSTEGKTSVKVKVVSEPKVKKAMPKSVSRAPKPLENPVSAKASTDTSRSVPSPAKSTPNSPVPTSASAPALTKSQTDRLEVLLNPKDEISLNSGKPFRELESELLSRRKKDLQQIYAEEREGGGSGGMTLDEEYLDITFLTENGFVRKRCPKCGKHFWSADPEREICGDPPCESYSFIGNPAFKNPFELDEMREYYLNFFERRGHGRIERYPVVAWWRTDIYLTIASIADFQPFVTSGVAPPPANPLTISQPCIRLDDLDCVGRTGRHLTLFEMMAHHAFNYPGKEIYWKNETVAYCTELLNELGVKKEDIVYKEELHAGGGNAGPCLEAIVGGLEVATLGFMNLEEHPEGDIEIKGARYRKMDNYIVDVGYGLERFVWASKGTPTVYDAIFPEVVDTIIDNSNVSFNREDDRVRRIVAESSKLAGIMGELRGERLNRLRKSVADIVGVSVEELEGIVVPLEKVYSLADHTRCILFMLGDGLVPSKAGAGYLARLMIRRSLRLAEELELGLDLYDLVEMHKKILGFEFDVPLSTVQEILELEKERYRTTVSKGTRLVERLVERKKKLEKDDLIELYCSHGIPVALAVGIAAEKGAEVEMPKDIYAELAKRHS

SEQ ID NO: 61 - protein, 696 aa, synthetic construct
  REGION 1..696: note = 61 AibRS-6
MDKKPLDVLISATGLWMSRTGTLHKIKHYEISRSKIYIEMACGDHLVVNNSRSCRPARAFRYHKYRKTCKRCRVSDEDINNFLTRSTEGKTSVKVKVVSEPKVKKAMPKSVSRAPKPLENPVSAKASTDTSRSVPSPAKSTPNSPVPTSASAPALTKSQTDRLEVLLNPKDEISLNSGKPFRELESELLSRRKKDLQQIYAEEREGGGSGGMTLDEEYLDITFLTENGFVRKRCPKCGKHFWSADPEREICGDPPCESYSFIGNPAFKNPFELDEMREYYLNFFERRGHGRIERYPVVAWWRTDIYLTIASIADFQPFVTSGVAPPPANPLTISQPCIRLDDLDCVGRTGRHLTLFEMMAHHAFNYPGKEIYWKNETVAYCTELLNELGVKKEDIVYKEELFAGGGNAGPCLEAIVGGLEVATLGFMNLEEHPEGDIEIKGARYRKMDNYIVDVGYGLERFVWASKGTPTVYDAIFPEVVDTIIDNSNVSFNREDDRVRRIVAESSKLAGIMGELRGERLNRLRKSVADIVGVSVEELEGIVVPLEKVYSLADHTRCILFMLGDGLVPSKAGAGYLARLMIRRSLRLAEELELGLDLYDLVEMHKKILGFEFDVPLSTVQEILELEKERYRTTVSKGTRLVERLVERKKKLEKDDLIELYCSHGIPVALAVGIAAEKGAEVEMPKDIYAELAKRHS

SEQ ID NO: 62 - protein, 696 aa, synthetic construct
  REGION 1..696: note = 62 AibRS-7
MDKKPLDVLISATGLWMSRTGTLHKIKHYEISRSKIYIEMACGDHLVVNNSRSCRPARAFRYHKYRKTCKRCRVSDEDINNFLTRSTEGKTSVKVKVVSEPKVKKAMPKSVSRAPKPLENPVSAKASTDTSRSVPSPAKSTPNSPVPTSASAPALTKSQTDRLEVLLNPKDEISLNSGKPFRELESELLSRRKKDLQQIYAEEREGGGSGGMTLDEEYLDITFLTENGFVRKRCPKCGKHFWSADPEREICGDPPCESYSFIGNPAFKNPFELDEMREYYLNFFERRGHGRIERYPVVAWWRTDIYLTIASIADFQPFVTSGVAPPPANPLTISQPCIRLDDLDCVGRTGRHLTLFEMMAHHAFNYPGKEIYWKNETVAYCTELLNELGVKKEDIVYKEELYAGGGNAGPCLEAIVGGLEVATLGFMNLEEHPEGDIEIKGARYRKMDNYIVDVGYGLERFVWASKGTPTVYDAIFPEVVDTIIDNSNVSFNREDDRVRRIVAESSKLAGIMGELRGERLNRLRKSVADIVGVSVEELEGIVVPLEKVYSLADHTRCILFMLGDGLVPSKAGAGYLARLMIRRSLRLAEELELGLDLYDLVEMHKKILGFEFDVPLSTVQEILELEKERYRTTVSKGTRLVERLVERKKKLEKDDLIELYCSHGIPVALAVGIAAEKGAEVEMPKDIYAELAKRHS

SEQ ID NO: 63 - protein, 696 aa, synthetic construct
  REGION 1..696: note = 63 AibRS-8
MDKKPLDVLISATGLWMSRTGTLHKIKHYEISRSKIYIEMACGDHLVVNNSRSCRPARAFRYHKYRKTCKRCRVSDEDINNFLTRSTEGKTSVKVKVVSEPKVKKAMPKSVSRAPKPLENPVSAKASTDTSRSVPSPAKSTPNSPVPTSASAPALTKSQTDRLEVLLNPKDEISLNSGKPFRELESELLSRRKKDLQQIYAEEREGGGSGGMTLDEEYLDITFLTENGFVRKRCPKCGKHFWSADPEREICGDPPCESYSFIGNPAFKNPFELDEMREYYLNFFERRGHGRIERYPVVAWWRTDIYLTIASIADFQPFVTSGVAPPPANPLTISQPCIRLDDLDCVGRTGRHLTLFEMMAHHAFNYPGKEIYWKNETVAYCTELLNELGVKKEDIVYKEELHAGGGNAGPCLEAIVGGLEVATLGFMNLEEHPEGDIEIKGARYRKMDNYIVDTGYGLERFVWASKGTPTVYDAIFPEVVDTIIDNSNVSFNREDDRVRRIVAESSKLAGIMGELRGERLNRLRKSVADIVGVSVEELEGIVVPLEKVYSLADHTRCILFMLGDGLVPSKAGAGYLARLMIRRSLRLAEELELGLDLYDLVEMHKKILGFEFDVPLSTVQEILELEKERYRTTVSKGTRLVERLVERKKKLEKDDLIELYCSHGIPVALAVGIAAEKGAEVEMPKDIYAELAKRHS

SEQ ID NO: 64 - protein, 696 aa, synthetic construct
  REGION 1..696: note = 64 AibRS-9
MDKKPLDVLISATGLWMSRTGTLHKIKHYEISRSKIYIEMACGDHLVVNNSRSCRPARAFRYHKYRKTCKRCRVSDEDINNFLTRSTEGKTSVKVKVVSEPKVKKAMPKSVSRAPKPLENPVSAKASTDTSRSVPSPAKSTPNSPVPTSASAPALTKSQTDRLEVLLNPKDEISLNSGKPFRELESELLSRRKKDLQQIYAEEREGGGSGGMTLDEEYLDITFLTENGFVRKRCPKCGKHFWSADPEREICGDPPCESYSFIGNPAFKNPFELDEMREYYLNFFERRGHGRIERYPVVAWWRTDIYLTIASIADFQPFVTSGVAPPPANPLTISQPCIRLDDLDCVGRTGRHLTLFEMMAHHAFNYPGKEIYWKNETVAYCTELLNELGVKKEDIVYKEELFAGGGNAGPCLEAIVGGLEVATLGFMNLEEHPEGDIEIKGARYRKMDNYIVDTGYGLERFVWASKGTPTVYDAIFPEVVDTIIDNSNVSFNREDDRVRRIVAESSKLAGIMGELRGERLNRLRKSVADIVGVSVEELEGIVVPLEKVYSLADHTRCILFMLGDGLVPSKAGAGYLARLMIRRSLRLAEELELGLDLYDLVEMHKKILGFEFDVPLSTVQEILELEKERYRTTVSKGTRLVERLVERKKKLEKDDLIELYCSHGIPVALAVGIAAEKGAEVEMPKDIYAELAKRHS

SEQ ID NO: 65 - protein, 696 aa, synthetic construct
  REGION 1..696: note = 65 AibRS-10
MDKKPLDVLISATGLWMSRTGTLHKIKHYEISRSKIYIEMACGDHLVVNNSRSCRPARAFRYHKYRKTCKRCRVSDEDINNFLTRSTEGKTSVKVKVVSEPKVKKAMPKSVSRAPKPLENPVSAKASTDTSRSVPSPAKSTPNSPVPTSASAPALTKSQTDRLEVLLNPKDEISLNSGKPFRELESELLSRRKKDLQQIYAEEREGGGSGGMTLDEEYLDITFLTENGFVRKRCPKCGKHFWSADPEREICGDPPCESYSFIGNPAFKNPFELDEMREYYLNFFERRGHGRIERYPVVAWWRTDIYLTIASIADFQPFVTSGVAPPPANPLTISQPCIRLDDLDCVGRTGRHLTLFEMMAHHAFNYPGKEIYWKNETVAYCTELLNELGVKKEDIVYKEELYAGGGNAGPCLEAIVGGLEVATLGFMNLEEHPEGDIEIKGARYRKMDNYIVDTGYGLERFVWASKGTPTVYDAIFPEVVDTIIDNSNVSFNREDDRVRRIVAESSKLAGIMGELRGERLNRLRKSVADIVGVSVEELEGIVVPLEKVYSLADHTRCILFMLGDGLVPSKAGAGYLARLMIRRSLRLAEELELGLDLYDLVEMHKKILGFEFDVPLSTVQEILELEKERYRTTVSKGTRLVERLVERKKKLEKDDLIELYCSHGIPVALAVGIAAEKGAEVEMPKDIYAELAKRHS

SEQ ID NO: 66 - protein, 696 aa, synthetic construct
  REGION 1..696: note = 66AibRS-11
MDKKPLDVLISATGLWMSRTGTLHKIKHYEISRSKIYIEMACGDHLVVNNSRSCRPARAFRYHKYRKTCKRCRVSDEDINNFLTRSTEGKTSVKVKVVSEPKVKKAMPKSVSRAPKPLENPVSAKASTDTSRSVPSPAKSTPNSPVPTSASAPALTKSQTDRLEVLLNPKDEISLNSGKPFRELESELLSRRKKDLQQIYAEEREGGGSGGMTLDEEYLDITFLTENGFVRKRCPKCGKHFWSADPEREICGDPPCESYSFIGNPAFKNPFELDEMREYYLNFFERRGHGRIERYPVVAWWRTDIYLTIASIADFQPFVTSGVAPPPANPLTISQPCIRLDDLDCVGRTGRHLTLFEMMAHHAFNYPGKEIYWKNETVAYCTELLNELGVKKEDIVYKEELLAGGGNAGPCLEAIVGGLEVATLGFMNLEEHPEGDIEIKGARYRKMDNYIVDVGYGLERFVWASKGTPTVYDAIFPEVVDTIIDNSNVSFNREDDRVRRIVAESSKLAGIMGELRGERLNRLRKSVADIVGVSVEELEGIVVPLEKVYSLADHTRCILFMLGDGLVPSKAGAGYLARLMIRRSLRLAEELELGLDLYDLVEMHKKILGFEFDVPLSTVQEILELEKERYRTTVSKGTRLVERLVERKKKLEKDDLIELYCSHGIPVALAVGIAAEKGAEVEMPKDIYAELAKRHS

SEQ ID NO: 67 - protein, 696 aa, synthetic construct
  REGION 1..696: note = 67 AibRS-12
MDKKPLDVLISATGLWMSRTGTLHKIKHYEISRSKIYIEMACGDHLVVNNSRSCRPARAFRYHKYRKTCKRCRVSDEDINNFLTRSTEGKTSVKVKVVSEPKVKKAMPKSVSRAPKPLENPVSAKASTDTSRSVPSPAKSTPNSPVPTSASAPALTKSQTDRLEVLLNPKDEISLNSGKPFRELESELLSRRKKDLQQIYAEEREGGGSGGMTLDEEYLDITFLTENGFVRKRCPKCGKHFWSADPEREICGDPPCESYSFIGNPAFKNPFELDEMREYYLNFFERRGHGRIERYPVVAWWRTDIYLTIASIADFQPFVTSGVAPPPANPLTISQPCIRLDDLDCVGRTGRHLTLFEMMAHHAFNYPGKEIYWKNETVAYCTELLNELGVKKEDIVYKEELIAGGGNAGPCLEAIVGGLEVATLGFMNLEEHPEGDIEIKGARYRKMDNYIVDVGYGLERFVWASKGTPTVYDAIFPEVVDTIIDNSNVSFNREDDRVRRIVAESSKLAGIMGELRGERLNRLRKSVADIVGVSVEELEGIVVPLEKVYSLADHTRCILFMLGDGLVPSKAGAGYLARLMIRRSLRLAEELELGLDLYDLVEMHKKILGFEFDVPLSTVQEILELEKERYRTTVSKGTRLVERLVERKKKLEKDDLIELYCSHGIPVALAVGIAAEKGAEVEMPKDIYAELAKRHS

SEQ ID NO: 68 - protein, 696 aa, synthetic construct
  REGION 1..696: note = 68 AibRS-13
MDKKPLDVLISATGLWMSRTGTLHKIKHYEISRSKIYIEMACGDHLVVNNSRSCRPARAFRYHKYRKTCKRCRVSDEDINNFLTRSTEGKTSVKVKVVSEPKVKKAMPKSVSRAPKPLENPVSAKASTDTSRSVPSPAKSTPNSPVPTSASAPALTKSQTDRLEVLLNPKDEISLNSGKPFRELESELLSRRKKDLQQIYAEEREGGGSGGMTLDEEYLDITFLTENGFVRKRCPKCGKHFWSADPEREICGDPPCESYSFIGNPAFKNPFELDEMREYYLNFFERRGHGRIERYPVVAWWRTDIYLTIASIADFQPFVTSGVAPPPANPLTISQPCIRLDDLDCVGRTGRHLTLFEMMAHHAFNYPGKEIYWKNETVAYCTELLNELGVKKEDIVYKEELMAGGGNAGPCLEAIVGGLEVATLGFMNLEEHPEGDIEIKGARYRKMDNYIVDVGYGLERFVWASKGTPTVYDAIFPEVVDTIIDNSNVSFNREDDRVRRIVAESSKLAGIMGELRGERLNRLRKSVADIVGVSVEELEGIVVPLEKVYSLADHTRCILFMLGDGLVPSKAGAGYLARLMIRRSLRLAEELELGLDLYDLVEMHKKILGFEFDVPLSTVQEILELEKERYRTTVSKGTRLVERLVERKKKLEKDDLIELYCSHGIPVALAVGIAAEKGAEVEMPKDIYAELAKRHS

SEQ ID NO: 69 - protein, 696 aa, synthetic construct
  REGION 1..696: note = 69 AibRS-14
MDKKPLDVLISATGLWMSRTGTLHKIKHYEISRSKIYIEMACGDHLVVNNSRSCRPARAFRYHKYRKTCKRCRVSDEDINNFLTRSTEGKTSVKVKVVSEPKVKKAMPKSVSRAPKPLENPVSAKASTDTSRSVPSPAKSTPNSPVPTSASAPALTKSQTDRLEVLLNPKDEISLNSGKPFRELESELLSRRKKDLQQIYAEEREGGGSGGMTLDEEYLDITFLTENGFVRKRCPKCGKHFWSADPEREICGDPPCESYSFIGNPAFKNPFELDEMREYYLNFFERRGHGRIERYPVVAWWRTDIYLTIASIADFQPFVTSGVAPPPANPLTISQPCIRLDDLDCVGRTGRHLTLFEMMAHHAFNYPGKEIYWKNETVAYCTELLNELGVKKEDIVYKEELHAGGGNAGPCLEAIVGGLEVATLAFMNLEEHPEGDIEIKGARYRKMDNYIVDVGYGLERFVWASKGTPTVYDAIFPEVVDTIIDNSNVSFNREDDRVRRIVAESSKLAGIMGELRGERLNRLRKSVADIVGVSVEELEGIVVPLEKVYSLADHTRCILFMLGDGLVPSKAGAGYLARLMIRRSLRLAEELELGLDLYDLVEMHKKILGFEFDVPLSTVQEILELEKERYRTTVSKGTRLVERLVERKKKLEKDDLIELYCSHGIPVALAVGIAAEKGAEVEMPKDIYAELAKRHS

SEQ ID NO: 70 - protein, 696 aa, synthetic construct
  REGION 1..696: note = 70 AibRS-15
MDKKPLDVLISATGLWMSRTGTLHKIKHYEISRSKIYIEMACGDHLVVNNSRSCRPARAFRYHKYRKTCKRCRVSDEDINNFLTRSTEGKTSVKVKVVSEPKVKKAMPKSVSRAPKPLENPVSAKASTDTSRSVPSPAKSTPNSPVPTSASAPALTKSQTDRLEVLLNPKDEISLNSGKPFRELESELLSRRKKDLQQIYAEEREGGGSGGMTLDEEYLDITFLTENGFVRKRCPKCGKHFWSADPEREICGDPPCESYSFIGNPAFKNPFELDEMREYYLNFFERRGHGRIERYPVVAWWRTDIYLTIASIADFQPFVTSGVAPPPANPLTISQPCIRLDDLDCVGRTGRHLTLFEMMAHHAFNYPGKEIYWKNETVAYCTELLNELGVKKEDIVYKEELFAGGGNAGPCLEAIVGGLEVASLGFMNLEEHPEGDIEIKGARYRKMDNYIVDVGYGLERFVWASKGTPTVYDAIFPEVVDTIIDNSNVSFNREDDRVRRIVAESSKLAGIMGELRGERLNRLRKSVADIVGVSVEELEGIVVPLEKVYSLADHTRCILFMLGDGLVPSKAGAGYLARLMIRRSLRLAEELELGLDLYDLVEMHKKILGFEFDVPLSTVQEILELEKERYRTTVSKGTRLVERLVERKKKLEKDDLIELYCSHGIPVALAVGIAAEKGAEVEMPKDIYAELAKRHS

SEQ ID NO: 71 - protein, 696 aa, synthetic construct
  REGION 1..696: note = 71 AibRS-16
MDKKPLDVLISATGLWMSRTGTLHKIKHYEISRSKIYIEMACGDHLVVNNSRSCRPARAFRYHKYRKTCKRCRVSDEDINNFLTRSTEGKTSVKVKVVSEPKVKKAMPKSVSRAPKPLENPVSAKASTDTSRSVPSPAKSTPNSPVPTSASAPALTKSQTDRLEVLLNPKDEISLNSGKPFRELESELLSRRKKDLQQIYAEEREGGGSGGMTLDEEYLDITFLTENGFVRKRCPKCGKHFWSADPEREICGDPPCESYSFIGNPAFKNPFELDEMREYYLNFFERRGHGRIERYPVVAWWRTDIYLTIASIADFQPFVTSGVAPPPANPLTISQPCIRLDDLDCVGRTGRHLTLFEMMAHHAFNYPGKEIYWKNETVAYCTELLNELGVKKEDIVYKEELFAGGGNAGPCLEAIVGGLEVATLGFMNLEEHPEGDIEIKGARYRKMDNYIVDLGYGLERFVWASKGTPTVYDAIFPEVVDTIIDNSNVSFNREDDRVRRIVAESSKLAGIMGELRGERLNRLRKSVADIVGVSVEELEGIVVPLEKVYSLADHTRCILFMLGDGLVPSKAGAGYLARLMIRRSLRLAEELELGLDLYDLVEMHKKILGFEFDVPLSTVQEILELEKERYRTTVSKGTRLVERLVERKKKLEKDDLIELYCSHGIPVALAVGIAAEKGAEVEMPKDIYAELAKRHS

SEQ ID NO: 72 - protein, 696 aa, synthetic construct
  REGION 1..696: note = 72 AibRS-17
MDKKPLDVLISATGLWMSRTGTLHKIKHYEISRSKIYIEMACGDHLVVNNSRSCRPARAFRYHKYRKTCKRCRVSDEDINNFLTRSTEGKTSVKVKVVSEPKVKKAMPKSVSRAPKPLENPVSAKASTDTSRSVPSPAKSTPNSPVPTSASAPALTKSQTDRLEVLLNPKDEISLNSGKPFRELESELLSRRKKDLQQIYAEEREGGGSGGMTLDEEYLDITFLTENGFVRKRCPKCGKHFWSADPEREICGDPPCESYSFIGNPAFKNPFELDEMREYYLNFFERRGHGRIERYPVVAWWRTDIYLTIASIADFQPFVTSGVAPPPANPLTISQPCIRLDDLDCVGRTGRHLTLFEMMAHHAFNYPGKEIYWKNETVAYCTELLNELGVKKEDIVYKEELFAGGGNAGPCLEAIVGGLEVATLGFMNLEEHPEGDIEIKGARYRKMDNYIVEVGYGLERFVWASKGTPTVYDAIFPEVVDTIIDNSNVSFNREDDRVRRIVAESSKLAGIMGELRGERLNRLRKSVADIVGVSVEELEGIVVPLEKVYSLADHTRCILFMLGDGLVPSKAGAGYLARLMIRRSLRLAEELELGLDLYDLVEMHKKILGFEFDVPLSTVQEILELEKERYRTTVSKGTRLVERLVERKKKLEKDDLIELYCSHGIPVALAVGIAAEKGAEVEMPKDIYAELAKRHS

SEQ ID NO: 73 - protein, 696 aa, synthetic construct
  REGION 1..696: note = 73 AibRS-18
MDKKPLDVLISATGLWMSRTGTLHKIKHYEISRSKIYIEMACGDHLVVNNSRSCRPARAFRYHKYRKTCKRCRVSDEDINNFLTRSTEGKTSVKVKVVSEPKVKKAMPKSVSRAPKPLENPVSAKASTDTSRSVPSPAKSTPNSPVPTSASAPALTKSQTDRLEVLLNPKDEISLNSGKPFRELESELLSRRKKDLQQIYAEEREGGGSGGMTLDEEYLDITFLTENGFVRKRCPKCGKHFWSADPEREICGDPPCESYSFIGNPAFKNPFELDEMREYYLNFFERRGHGRIERYPVVAWWRTDIYLTIASIADFQPFVTSGVAPPPANPLTISQPCIRLDDLDCVGRTGRHLTLFEMMAHHAFNYPGKEIYWKNETVAYCTELLNELGVKKEDIVYKEELHAGGGNAGPCLEAIVGGLEVAGLGFMNLEEHPEGDIEIKGARYRKMDNYIVDVGYGLERFVWASKGTPTVYDAIFPEVVDTIIDNSNVSFNREDDRVRRIVAESSKLAGIMGELRGERLNRLRKSVADIVGVSVEELEGIVVPLEKVYSLADHTRCILFMLGDGLVPSKAGAGYLARLMIRRSLRLAEELELGLDLYDLVEMHKKILGFEFDVPLSTVQEILELEKERYRTTVSKGTRLVERLVERKKKLEKDDLIELYCSHGIPVALAVGIAAEKGAEVEMPKDIYAELAKRHS

SEQ ID NO: 74 - protein, 556 aa, synthetic construct
  REGION 1..556: note = 74 AibRS-19
MSKSTAEIRQAFLDFFHSKGHQVVASSSLVPHNDPTLLFTNAGMNQFKDVFLGLDKRNYSRATTSQRCVRAGGKHNDLENVGYTARHHTFFEMLGNFSFGDYFKHDAIQFAWELLTSEKWFALPKERLWVTVYESDDEAYEIWEKEVGIPRERIIRIGDNKGAPYASDNFHIMGDTGPCGPCTEIFYDHGDHIWGGPPGSPEEDGDRYIEIWNIGFIQFNRQADGTMEPLPKPSVDTGMGLERIAAVLQHVNSNYDIDLFRTLIQAVAKVTGATDLSNKSLRVIADHIRSCAFLIADGVMPSNENRGYVLRRIIRRAVRHGNMLGAKETFFYKLVGPLIDVMGSAGEDLKRQQAQVEQVLKTEEEQFARTLERGLALLDEELAGGGSGGGSGGGSGGGSGGGSGGGSMDKKPLDVLISATGLWMSRTGTLHKIKHYEISRSKIYIEMACGDHLVVNNSRSCRPARAFRYHKYRKTCKRCRVSDEDINNFLTRSTEGKTSVKVKVVSEPKVKKAMPKSVSRAPKPLENPVSAKASTDTSRSVPSPAKSTPNSPVPTS

SEQ ID NO: 75 - protein, 716 aa, synthetic construct
  REGION 1..716: note = 75 AibRS-20
MDKKPLDVLISATGLWMSRTGTLHKIKHYEISRSKIYIEMACGDHLVVNNSRSCRPARAFRYHKYRKTCKRCRVSDEDINNFLTRSTEGKTSVKVKVVSEPKVKKAMPKSVSRAPKPLENPVSAKASTDTSRSVPSPAKSTPNSPVPTSASAPALTKSQTDRLEVLLNPKDEISLNSGKPFRELESELLSRRKKDLQQIYAEEREGGGSGGMTLDEEYLDITFLTENGFVRKRCPKCGKHFWTADPEREICGDPPCESYSFIGNPVFKKPFELDMSKSTAEIRQAFLDFFHSKGHQVVASSSLVPHNDPTLLFTNAGMNQFKDVFLGLDKRNYSRATTSQRCVRAGGKHNDLENVGYTARHHTFFEMLGNFSFGDYFKHDAIQFAWELLTSEKWFALPKERLWVTVYESDDEAYEIWEKEVGIPRERIIRIGDNKGAPYASDNFHQMGDTGPCGPCTEIFYDHGDHIWGGPPGSPEEDGDRYIEIWNIGFMQFNRQADGTMEPLPKPSVDVGMGLERIAAVLQHVNSNYDIDLFRTLIQAVAKVTGATDLSNKSLRVIADHIRSCAFLIADGVMPSKENRGYVLRRIIRRAVRHGNMLGAKETFFYKLVGPLIDVMGSAGEDLKRQQAQVEQVLKTEEEQFARTLERGLALLDEELAKLSGDTLDGETAFRLYRTYGFPVALTADVCRERNIKVDEAGFEAAMEEQRRRAREASGF

SEQ ID NO: 76 - protein, 31 aa, synthetic construct
  REGION 1..31: note = 76 sema
  MOD_RES 2: note = X is Aib
HXEGTFTSDVSSYLEGQAAKEFIAWLVRGRG

SEQ ID NO: 77 - protein, 31 aa, synthetic construct; identical to SEQ ID NO: 76
  REGION 1..31: note = 77 sema_dmeC
  MOD_RES 2: note = X is D-methylcysteine

SEQ ID NO: 78 - protein, 31 aa, synthetic construct; identical to SEQ ID NO: 76
  REGION 1..30: note = 78 sema_dmeS
  MOD_RES 2: note = X is D-methylserine

SEQ ID NO: 79 - protein, 31 aa, synthetic construct; identical to SEQ ID NO: 76
  REGION 1..31: note = 79 sema_D-isoVal
  MOD_RES 2: note = X is D-isovaline

SEQ ID NO: 80 - protein, 39 aa, synthetic construct
  REGION 1..37: note = 80 Tir
  MOD_RES 2: note = X is Aib
  MOD_RES 13: note = X is Aib
YXEGTFTSDYSIXLDKIAQKAFVQWLIAGGPSSGAPPPS

SEQ ID NO: 81 - DNA, 444 bp, synthetic construct
  misc_feature 1..444: note = 81 sumo-glp1-8tag
  source 1..444: mol_type = other DNA
atgggcagcagccatcatcatcatcatcacagcagcggcctggtgccgcgcggcagcatgtcggactcagaagtcaatcaagaagctaagccagaggtcaagccagaagtcaagcctgagactcacatcaatttaaaggtgtccgatggatcttcagagatcttcttcaagatcaaaaagaccactcctttaagaaggctgatggaagcgttcgctaaaagacagggtaaggaaatggactccttaagattcttgtacgacggtattagaattcaagctgatcagacccctgaagatttggacatggaggataacgatattattgaggctcacagagaacagattggtggacattaggaaggcacctttaccagcgatgtgagcagctatctggaaggccaggcggcgaaagaatttattgcgtggctggtgaggggccgcggc

SEQ ID NO: 82 - DNA, 171 bp, synthetic construct
  misc_feature 1..171: note = 82 mae-glp-8tag
  source 1..171: mol_type = other DNA
atgaaaattaaaaccggcgcgcgcattctggcgctgagcgcgctgaccaccatgatgtttagcgctagcgcgctggcccatgccgaaggcacgttcacctcggatgtttcaagctatttagagggccaggcagcgaaagaatttattgcgtggctggtgcgtggtcgcgga

SEQ ID NO: 83 - DNA, 243 bp, synthetic construct
  misc_feature 1..243: note = 83 2*
  source 1..243: mol_type = other DNA
atgggccaccaccaccatgaagaagcggaggcagaagctcgcggtaaacgtcactaggaaggcacgttcacctctgacgtaagctcttacctggagggtcaggcggccaaagaatttattgcgtggctggttcgtggtcgtggtaaacgtcactaggaaggtaccttcactagcgacgtttcttcctatctggagggccaggcggcaaaagaattcattgcgtggctggtacgtggccgtggt

SEQ ID NO: 84 - DNA, 342 bp, synthetic construct
  misc_feature 1..342: note = 84 3*
  source 1..342: mol_type = other DNA
atgggccaccaccaccacgaagaagctgaagctgaagcgcgcggtaaacgccactaggagggtaccttcacctctgacgtgtcctcttacctggaaggtcaggcagccaaagagttcatcgcttggctggtacgtggtcgcggtaaacgtcattaggaaggcaccttcacctctgatgtatcttcctatctggagggtcaagctgctaaggaattcattgcgtggctggttcgtggtcgtggtaaacgtcactaggaaggtaccttcactagcgatgtgtccagctacctggaaggtcaggcggctaaagagttcatcgcatggctggtacgtggccgcggt

SEQ ID NO: 85 - DNA, 441 bp, synthetic construct
  misc_feature 1..441: note = 85 4*
  source 1..441: mol_type = other DNA
atgggccaccaccaccatgaagaagctgaagctgaagcgcgcggtaaacgtcactaggagggtaccttcacctctgatgtttcctcttacctggagggtcaggcggccaaagaattcattgcttggctggttcgtggtcgcggcaaacgtcactaggaaggtaccttcaccagcgatgtctccagctatctggaaggccaggcggcaaaagaatttatcgcctggctggtgcgtggtcgcggtaaacgtcactaggaaggtacgtttactagcgacgttagctcctacctggaaggccaggcggcgaaagaatttatcgcgtggctggtgcgtggtcgtggcaaacgccattaggaaggcacgttcacttctgatgttagctcctatctggaaggtcaggcagcgaaagaattcattgcgtggctggtacgcggccgtggt

SEQ ID NO: 86 - protein, 103 aa, synthetic construct
  REGION 1..103: note = TD1
MDKKPLDVLISATGLWMSRTGTLHKIKHYEISRSKIYIEMACGDHLVVNNSRSCRPARAFRYHKYRKTCKRCRVSDEDINNFLTRSTEGKTSVKVKVVSEPKV

SEQ ID NO: 87 - protein, 149 aa, synthetic construct
  REGION 1..149: note = TD2
MDKKPLDVLISATGLWMSRTGTLHKIKHYEISRSKIYIEMACGDHLVVNNSRSCRPARAFRYHKYRKTCKRCRVSDEDINNFLTRSTEGKTSVKVKVVSEPKVKKAMPKSVSRAPKPLENPVSAKASTDTSRSVPSPAKSTPNSPVPTS

SEQ ID NO: 88 - protein, 446 aa, synthetic construct
  REGION 1..446: note = CD1
AWRGLSHRPLSSEPPAAKASAVRAAFLNFFRDRHGHRLVPSASVRPRGDPSLLFVNAGMNQFKPIFLGTVDPRSEMAGFRRVANSQKCVRAGGHHNDLEDVGRDLSHHTFFEMLGNWAFGGEYFKEEACNMAWELLTQVYGIPEERLWISYFDGDPKAGLDPDLETRDIWLSLGVPASRVLSFGPQENFWEMGDTGPCGPCTEIHYDLAGGVGAPQLVELWNLVFMQHNREADGSLQPLPQRHVDTGMGLERLVAVLQGKHSTYDTDLFSPLLNAIQQGCRAPPYLGRVGVADEGRTDTAYRVVADHIRTLSVCISDGIFPGMSGPPLVLRRILRRAVRFSMEILKAPPGFLGSLVPVVVETLGDAYPELQRNSAQIANLVSEDEAAFLASLERGRRIIDRTLRTLGPSDMFPAEVAWSLSLCGDLGLPLDMVELMLEEKGVQLDS

SEQ ID NO: 89 - protein, 488 aa, synthetic construct
  REGION 1..488: note = CD2
LEDEYQLDFFSENGFVRKQCSKCGKFYWTRDLDRNTCGDAPCDPYSFIGNPVFNKKFDLAEMREYYLKFFEDQGHTRLERYPVVARWRDDIYLTIASIADFQPFVTSGQVRPPANPLTISQPCIRLSDLDAVGRSGRHLTTFEMMAHHAFNKKGEEIYWKDRTMELCDELFNSLGVDPLAVTYKEEPWAGGGNAGPCVEALIGGLEVATLVFMDLEQSKDGTIPIKGENYRKMDNYIVDTGYGLERFVWASKGSPTIYDAVFPTIVNELMQLAGLEHQIDDPEYANILSQSARLAGLMDVSEKANLLQLRKQVASSIGTSVEKLSSIMEPVENIYAITDHTRCLTFMLADGIIPSNVKAGYLARLVLRRTLRMMKDMDINTSLSDIVRMHIKNLPEYPEFSEKFDVIEDILAHEEMKFDETLERGRRMIQKSAKHYKSTGEKIPLPTLIEMYASHGIPPEISKEAASEMGVEVDLPDNFYSLVAEGHS

SEQ ID NO: 90 - protein, 471 aa, synthetic construct
  REGION 1..471: note = CD3
LSTKLFETAGFHRKQCPMCKSYFWTLRQDQVYCGDQPCVPYGFIGNSPVRVPVESIRDLRERFLSFFEKRGHARIKRYPVVARWREDVFLVGASIYDFQPWVTSGAVPPPANPLTISQPSIRLTDVDKVGRSGRHLTGFEMMAHHAFNYPDKHIYWISETTEYAYEFFTKELGIPEDEITFKESVWEGGGNAGESFEVLVRGLEVATLVFMHYEVKDGKYVELPLKIVDTGYGLERIYWMAKGSPTVYDAVFGPFLSAARKKLGVPEPPAHILGKASVYFGQMDPEVIGLAKAYDLVAEKIGIDPREFREIMRPQEALYVLADHSRTVSWMIADGVIPSNTGAGYLARLLIRRALKSLWIAGIETSLVDLFDLHLGFLRDDYPEVWQSRDIILELIDLEEKKYKDVIKSAPAAVRRALEKAGGKSLKAEQLVELYASYGIPPEVAQEVARKLGAEAEVPDDFYSRLAARHQ

SEQ ID NO: 91 - protein, 486 aa, synthetic construct
  REGION 1..486: note = CD4
MDMTTRMFKEEGWIRKQCPKCGKFFWTLDPDRETCGDPPCDEYSFIGKPGIPKKYTLEEMREAFLRFFEKHDHGRVKRFPVLPRWRDDVLLVGASIMDFQPWVISGEADPPANPLTISQPSIRFTDIDNVGITGRHFTIFEMMAHHAFNYPGKPIYWMDETVELAFEFFTKELGMKAEDITFKENPWAGGGNAGPAFEVLYRGLEVATLVFMQYKKAPENADPSQVVEIKGDYYVPMETKVVDTGYGLERLVWMSHGTPTAYDAVLGYVVEPLKRMAGIERIDERILMENSRLAGMFDIEDMGDLRYLRGQVAKRVGISVEELEKAVRPYELIYAIADHTKALTFMLADGVIPSNVKAGYLARLLIRKSIRHLRELGLEVPLAEIVAMHIKELSPTFPEFKEMEDVILDIINVEEKRYQETLKRGSDLVKREIARLKKAGKNEMPLEKLILFYASHGLTPEIVREVAEKEGVKVEIPDNFYTLVAK

SEQ ID NO: 92 - protein, 492 aa, synthetic construct
  REGION 1..492: note = CD5
MTLDEEYLDITFLTENGFVRKRCPKCGKHFWTADPEREICGDPPCESYSFIGNPVVSQPFRSADIRQAFIDFFISKQHTPVASSSLIPHNDPTLLFTNAGMNQFKETFLGMEPRDYTRAVTSQKCVRAGGKHNDLDNVGYTARHHTFFEMLGNFSFGDYFKQAGIGYIWEFLTSDEWLAIDKNRLYVTIYETDDEAFDIWHKDIGIPSERIIRIGDNKGAPYASDNFWTMGDTGPCGPCTEVFYDHGADIEGGLPGTPEEDGDRYIEIWNCVFMQFNRQKDGTMLPLPAPSVDTGMGLERISAIMQGVHGNYEIDLFVHLMDAAAEILEIENQQQSSLKVIADHIRAVSFLIADGVTPSNEGRGYVLRRVIRRAVRHGNKLGADSDFFYKMVAPLVAEMGIAYPELKDKQSVIENTIQKEEAQFAKTLAQGLRLLASELEGLQDGDTLSGEAAFKLYDTYGFPVDLTADITRERGIVIDEAEFDEHMQAQRE

SEQ ID NO: 93 - protein, 383 aa, synthetic construct
  REGION 1..383: note = CD6
MSKSTAEIRQAFLDFFHSKGHQVVASSSLVPHNDPTLLFTNAGMNQFKDVFLGLDKRNYSRATTSQRCVRAGGKHNDLENVGYTARHHTFFEMLGNFSFGDYFKHDAIQFAWELLTSEKWFALPKERLWVTVYESDDEAYEIWEKEVGIPRERIIRIGDNKGAPYASDNFHQMGDTGPCGPCTEIFYDHGDHIWGGPPGSPEEDGDRYIEIWNIGFMQFNRQADGTMEPLPKPSVDVGMGLERIAAVLQHVNSNYDIDLFRTLIQAVAKVTGATDLSNKSLRVIADHIRSCAFLIADGVMPSNENRGYVLRRIIRRAVRHGNMLGAKETFFYKLVGPLIDVMGSAGEDLKRQQAQVEQVLKTEEEQFARTLERGLALLDEELA

SEQ ID NO: 94 - protein, 485 aa, synthetic construct
  REGION 1..485: note = CD7
MTLDEEYLDITFLTENGFVRKRCPKCGKHFWTADPEREICGDPPCESYSFIGNPVFKKPFELDEMREYYLNFFERRGHGRIERYPVVARWRTDIYLTIASIADFQPFVTSGVAPPPANPLTISQPCIRLDDLDSVGRTGRHLTLFEMMAHHAFNYPGKEIYWKNETVAYCTELLNELGVKKEDIVYKEEPHAGGGNAGPCLEAIVGGLEVATLGFMNLEEHPEGDIEIKGARYRKMDNYIVDVGYGLERFVWASKGTPTVYDAIFPEVVDTIIDNSNVSFNREDERVRRIVAESSKLAGIMGELRGERLNQLRKSVADTVGVSVEELEGIVVPLEKVYSLADHTRCILFMLGDGLVPSNAGAGYLARLMIRRSLRLAEELELGLDLYDLVEMHKKILGFEFDVPLSTVQEILELEKERYRTTVSKGTRLVERLVERKKKLEKDDLIELYASHGIPVELAVGIAAEKGAEVEMPKDIYAELAKRHS

SEQ ID NO: 95 - protein, 485 aa, synthetic construct
  REGION 1..485: note = CD8
MTLDEEYLDITFLTENGFVRKRCPKCGKHFWTADPEREICGDPPCESYSFIGNPVFKKPFELDEMREYYLNFFERRGHGRIERYPVVARWRTDIYLTIASIADFQPFVTSGVAPPPANPLTISQPCIRLDDLDSVGRTGRHLTLFEMMAHHAFNYPGKEIYWKNETVAYCTELLNELGVKKEDIVYKEEPHAGGGNAGPCLEAIVGGLEVATLGFMNLEEHPEGDIEIKGARYRKMDNYIVDVGYGLERFVWASKGTPTVYDAIFPEVVDTIIDNSNVSFNREDERVRRIVAESSKLAGIMGELRGERLNQLRKSVADTVGVSVEELEGIVVPLEKVYSLADHTRCILFMLGDGLVPSAAGAGYLARLMIRRSLRLAEELELGLDLYDLVEMHKKILGFEFDVPLSTVQEILELEKERYRTTVSKGTRLVERLVERKKKLEKDDLIELYASHGIPVALAVGIAAEKGAEVEMPKDIYAELAKRHS

SEQ ID NO: 96 - protein, 485 aa, synthetic construct
  REGION 1..485: note = CD9
MTLDEEYLDITFLTENGFVRKRCPKCGKHFWSADPEREICGDPPCESYSFIGNPAFKNPFELDEMREYYLNFFERRGHGRIERYPVVAWWRTDIYLTIASIADFQPFVTSGVAPPPANPLTISQPCIRLDDLDCVGRTGRHLTLFEMMAHHAFNYPGKEIYWKNETVAYCTELLNELGVKKEDIVYKEELHAGGGNAGPCLEAIVGGLEVATLGFMNLEEHPEGDIEIKGARYRKMDNYIVDVGYGLERFVWASKGTPTVYDAIFPEVVDTIIDNSNVSFNREDDRVRRIVAESSKLAGIMGELRGERLNRLRKSVADIVGVSVEELEGIVVPLEKVYSLADHTRCILFMLGDGLVPSKAGAGYLARLMIRRSLRLAEELELGLDLYDLVEMHKKILGFEFDVPLSTVQEILELEKERYRTTVSKGTRLVERLVERKKKLEKDDLIELYCSHGIPVALAVGIAAEKGAEVEMPKDIYAELAKRHS

SEQ ID NO: 97 - protein, 485 aa, synthetic construct
  REGION 1..485: note = CD10
MTLDEEYLDITFLTENGFVRKRCPKCGKHFWSADPEREICGDPPCESYSFIGNPAFKNPFELDEMREYYLNFFERRGHGRIERYPVVAWWRTDIYLTIASIADFQPFVTSGVAPPPANPLTISQPCIRLDDLDCVGRTGRHLTLFEMMAHHAFNYPGKEIYWKNETVAYCTELLNELGVKKEDIVYKEELFAGGGNAGPCLEAIVGGLEVATLGFMNLEEHPEGDIEIKGARYRKMDNYIVDVGYGLERFVWASKGTPTVYDAIFPEVVDTIIDNSNVSFNREDDRVRRIVAESSKLAGIMGELRGERLNRLRKSVADIVGVSVEELEGIVVPLEKVYSLADHTRCILFMLGDGLVPSKAGAGYLARLMIRRSLRLAEELELGLDLYDLVEMHKKILGFEFDVPLSTVQEILELEKERYRTTVSKGTRLVERLVERKKKLEKDDLIELYCSHGIPVALAVGIAAEKGAEVEMPKDIYAELAKRHS

SEQ ID NO: 98 - protein, 485 aa, synthetic construct
  REGION 1..485: note = CD11
MTLDEEYLDITFLTENGFVRKRCPKCGKHFWSADPEREICGDPPCESYSFIGNPAFKNPFELDEMREYYLNFFERRGHGRIERYPVVAWWRTDIYLTIASIADFQPFVTSGVAPPPANPLTISQPCIRLDDLDCVGRTGRHLTLFEMMAHHAFNYPGKEIYWKNETVAYCTELLNELGVKKEDIVYKEELYAGGGNAGPCLEAIVGGLEVATLGFMNLEEHPEGDIEIKGARYRKMDNYIVDVGYGLERFVWASKGTPTVYDAIFPEVVDTIIDNSNVSFNREDDRVRRIVAESSKLAGIMGELRGERLNRLRKSVADIVGVSVEELEGIVVPLEKVYSLADHTRCILFMLGDGLVPSKAGAGYLARLMIRRSLRLAEELELGLDLYDLVEMHKKILGFEFDVPLSTVQEILELEKERYRTTVSKGTRLVERLVERKKKLEKDDLIELYCSHGIPVALAVGIAAEKGAEVEMPKDIYAELAKRHS

SEQ ID NO: 99 - protein, 485 aa, synthetic construct
  REGION 1..485: note = CD12
MTLDEEYLDITFLTENGFVRKRCPKCGKHFWSADPEREICGDPPCESYSFIGNPAFKNPFELDEMREYYLNFFERRGHGRIERYPVVAWWRTDIYLTIASIADFQPFVTSGVAPPPANPLTISQPCIRLDDLDCVGRTGRHLTLFEMMAHHAFNYPGKEIYWKNETVAYCTELLNELGVKKEDIVYKEELHAGGGNAGPCLEAIVGGLEVATLGFMNLEEHPEGDIEIKGARYRKMDNYIVDTGYGLERFVWASKGTPTVYDAIFPEVVDTIIDNSNVSFNREDDRVRRIVAESSKLAGIMGELRGERLNRLRKSVADIVGVSVEELEGIVVPLEKVYSLADHTRCILFMLGDGLVPSKAGAGYLARLMIRRSLRLAEELELGLDLYDLVEMHKKILGFEFDVPLSTVQEILELEKERYRTTVSKGTRLVERLVERKKKLEKDDLIELYCSHGIPVALAVGIAAEKGAEVEMPKDIYAELAKRHS

SEQ ID NO: 100 - protein, 485 aa, synthetic construct
  REGION 1..485: note = CD13
MTLDEEYLDITFLTENGFVRKRCPKCGKHFWSADPEREICGDPPCESYSFIGNPAFKNPFELDEMREYYLNFFERRGHGRIERYPVVAWWRTDIYLTIASIADFQPFVTSGVAPPPANPLTISQPCIRLDDLDCVGRTGRHLTLFEMMAHHAFNYPGKEIYWKNETVAYCTELLNELGVKKEDIVYKEELFAGGGNAGPCLEAIVGGLEVATLGFMNLEEHPEGDIEIKGARYRKMDNYIVDTGYGLERFVWASKGTPTVYDAIFPEVVDTIIDNSNVSFNREDDRVRRIVAESSKLAGIMGELRGERLNRLRKSVADIVGVSVEELEGIVVPLEKVYSLADHTRCILFMLGDGLVPSKAGAGYLARLMIRRSLRLAEELELGLDLYDLVEMHKKILGFEFDVPLSTVQEILELEKERYRTTVSKGTRLVERLVERKKKLEKDDLIELYCSHGIPVALAVGIAAEKGAEVEMPKDIYAELAKRHS

SEQ ID NO: 101 - protein, 485 aa, synthetic construct
  REGION 1..485: note = CD14
MTLDEEYLDITFLTENGFVRKRCPKCGKHFWSADPEREICGDPPCESYSFIGNPAFKNPFELDEMREYYLNFFERRGHGRIERYPVVAWWRTDIYLTIASIADFQPFVTSGVAPPPANPLTISQPCIRLDDLDCVGRTGRHLTLFEMMAHHAFNYPGKEIYWKNETVAYCTELLNELGVKKEDIVYKEELYAGGGNAGPCLEAIVGGLEVATLGFMNLEEHPEGDIEIKGARYRKMDNYIVDTGYGLERFVWASKGTPTVYDAIFPEVVDTIIDNSNVSFNREDDRVRRIVAESSKLAGIMGELRGERLNRLRKSVADIVGVSVEELEGIVVPLEKVYSLADHTRCILFMLGDGLVPSKAGAGYLARLMIRRSLRLAEELELGLDLYDLVEMHKKILGFEFDVPLSTVQEILELEKERYRTTVSKGTRLVERLVERKKKLEKDDLIELYCSHGIPVALAVGIAAEKGAEVEMPKDIYAELAKRHS

SEQ ID NO: 102 - protein, 485 aa, synthetic construct
  REGION 1..485: note = CD15
MTLDEEYLDITFLTENGFVRKRCPKCGKHFWSADPEREICGDPPCESYSFIGNPAFKNPFELDEMREYYLNFFERRGHGRIERYPVVAWWRTDIYLTIASIADFQPFVTSGVAPPPANPLTISQPCIRLDDLDCVGRTGRHLTLFEMMAHHAFNYPGKEIYWKNETVAYCTELLNELGVKKEDIVYKEELLAGGGNAGPCLEAIVGGLEVATLGFMNLEEHPEGDIEIKGARYRKMDNYIVDVGYGLERFVWASKGTPTVYDAIFPEVVDTIIDNSNVSFNREDDRVRRIVAESSKLAGIMGELRGERLNRLRKSVADIVGVSVEELEGIVVPLEKVYSLADHTRCILFMLGDGLVPSKAGAGYLARLMIRRSLRLAEELELGLDLYDLVEMHKKILGFEFDVPLSTVQEILELEKERYRTTVSKGTRLVERLVERKKKLEKDDLIELYCSHGIPVALAVGIAAEKGAEVEMPKDIYAELAKRHS

SEQ ID NO: 103 - protein, 485 aa, synthetic construct
  REGION 1..485: note = CD16
MTLDEEYLDITFLTENGFVRKRCPKCGKHFWSADPEREICGDPPCESYSFIGNPAFKNPFELDEMREYYLNFFERRGHGRIERYPVVAWWRTDIYLTIASIADFQPFVTSGVAPPPANPLTISQPCIRLDDLDCVGRTGRHLTLFEMMAHHAFNYPGKEIYWKNETVAYCTELLNELGVKKEDIVYKEELIAGGGNAGPCLEAIVGGLEVATLGFMNLEEHPEGDIEIKGARYRKMDNYIVDVGYGLERFVWASKGTPTVYDAIFPEVVDTIIDNSNVSFNREDDRVRRIVAESSKLAGIMGELRGERLNRLRKSVADIVGVSVEELEGIVVPLEKVYSLADHTRCILFMLGDGLVPSKAGAGYLARLMIRRSLRLAEELELGLDLYDLVEMHKKILGFEFDVPLSTVQEILELEKERYRTTVSKGTRLVERLVERKKKLEKDDLIELYCSHGIPVALAVGIAAEKGAEVEMPKDIYAELAKRHS

SEQ ID NO: 104 - protein, 485 aa, synthetic construct
  REGION 1..485: note = CD17
MTLDEEYLDITFLTENGFVRKRCPKCGKHFWSADPEREICGDPPCESYSFIGNPAFKNPFELDEMREYYLNFFERRGHGRIERYPVVAWWRTDIYLTIASIADFQPFVTSGVAPPPANPLTISQPCIRLDDLDCVGRTGRHLTLFEMMAHHAFNYPGKEIYWKNETVAYCTELLNELGVKKEDIVYKEELMAGGGNAGPCLEAIVGGLEVATLGFMNLEEHPEGDIEIKGARYRKMDNYIVDVGYGLERFVWASKGTPTVYDAIFPEVVDTIIDNSNVSFNREDDRVRRIVAESSKLAGIMGELRGERLNRLRKSVADIVGVSVEELEGIVVPLEKVYSLADHTRCILFMLGDGLVPSKAGAGYLARLMIRRSLRLAEELELGLDLYDLVEMHKKILGFEFDVPLSTVQEILELEKERYRTTVSKGTRLVERLVERKKKLEKDDLIELYCSHGIPVALAVGIAAEKGAEVEMPKDIYAELAKRHS

SEQ ID NO: 105 - protein, 485 aa, synthetic construct
  REGION 1..485: note = CD18
MTLDEEYLDITFLTENGFVRKRCPKCGKHFWSADPEREICGDPPCESYSFIGNPAFKNPFELDEMREYYLNFFERRGHGRIERYPVVAWWRTDIYLTIASIADFQPFVTSGVAPPPANPLTISQPCIRLDDLDCVGRTGRHLTLFEMMAHHAFNYPGKEIYWKNETVAYCTELLNELGVKKEDIVYKEELHAGGGNAGPCLEAIVGGLEVATLAFMNLEEHPEGDIEIKGARYRKMDNYIVDVGYGLERFVWASKGTPTVYDAIFPEVVDTIIDNSNVSFNREDDRVRRIVAESSKLAGIMGELRGERLNRLRKSVADIVGVSVEELEGIVVPLEKVYSLADHTRCILFMLGDGLVPSKAGAGYLARLMIRRSLRLAEELELGLDLYDLVEMHKKILGFEFDVPLSTVQEILELEKERYRTTVSKGTRLVERLVERKKKLEKDDLIELYCSHGIPVALAVGIAAEKGAEVEMPKDIYAELAKRHS

SEQ ID NO: 106 - protein, 485 aa, synthetic construct
  REGION 1..485: note = CD19
MTLDEEYLDITFLTENGFVRKRCPKCGKHFWSADPEREICGDPPCESYSFIGNPAFKNPFELDEMREYYLNFFERRGHGRIERYPVVAWWRTDIYLTIASIADFQPFVTSGVAPPPANPLTISQPCIRLDDLDCVGRTGRHLTLFEMMAHHAFNYPGKEIYWKNETVAYCTELLNELGVKKEDIVYKEELFAGGGNAGPCLEAIVGGLEVASLGFMNLEEHPEGDIEIKGARYRKMDNYIVDVGYGLERFVWASKGTPTVYDAIFPEVVDTIIDNSNVSFNREDDRVRRIVAESSKLAGIMGELRGERLNRLRKSVADIVGVSVEELEGIVVPLEKVYSLADHTRCILFMLGDGLVPSKAGAGYLARLMIRRSLRLAEELELGLDLYDLVEMHKKILGFEFDVPLSTVQEILELEKERYRTTVSKGTRLVERLVERKKKLEKDDLIELYCSHGIPVALAVGIAAEKGAEVEMPKDIYAELAKRHS

SEQ ID NO: 107 - protein, 485 aa, synthetic construct
  REGION 1..485: note = CD20
MTLDEEYLDITFLTENGFVRKRCPKCGKHFWSADPEREICGDPPCESYSFIGNPAFKNPFELDEMREYYLNFFERRGHGRIERYPVVAWWRTDIYLTIASIADFQPFVTSGVAPPPANPLTISQPCIRLDDLDCVGRTGRHLTLFEMMAHHAFNYPGKEIYWKNETVAYCTELLNELGVKKEDIVYKEELFAGGGNAGPCLEAIVGGLEVATLGFMNLEEHPEGDIEIKGARYRKMDNYIVDLGYGLERFVWASKGTPTVYDAIFPEVVDTIIDNSNVSFNREDDRVRRIVAESSKLAGIMGELRGERLNRLRKSVADIVGVSVEELEGIVVPLEKVYSLADHTRCILFMLGDGLVPSKAGAGYLARLMIRRSLRLAEELELGLDLYDLVEMHKKILGFEFDVPLSTVQEILELEKERYRTTVSKGTRLVERLVERKKKLEKDDLIELYCSHGIPVALAVGIAAEKGAEVEMPKDIYAELAKRHS

SEQ ID NO: 108 - protein, 485 aa, synthetic construct
  REGION 1..485: note = CD21
MTLDEEYLDITFLTENGFVRKRCPKCGKHFWSADPEREICGDPPCESYSFIGNPAFKNPFELDEMREYYLNFFERRGHGRIERYPVVAWWRTDIYLTIASIADFQPFVTSGVAPPPANPLTISQPCIRLDDLDCVGRTGRHLTLFEMMAHHAFNYPGKEIYWKNETVAYCTELLNELGVKKEDIVYKEELFAGGGNAGPCLEAIVGGLEVATLGFMNLEEHPEGDIEIKGARYRKMDNYIVEVGYGLERFVWASKGTPTVYDAIFPEVVDTIIDNSNVSFNREDDRVRRIVAESSKLAGIMGELRGERLNRLRKSVADIVGVSVEELEGIVVPLEKVYSLADHTRCILFMLGDGLVPSKAGAGYLARLMIRRSLRLAEELELGLDLYDLVEMHKKILGFEFDVPLSTVQEILELEKERYRTTVSKGTRLVERLVERKKKLEKDDLIELYCSHGIPVALAVGIAAEKGAEVEMPKDIYAELAKRHS

SEQ ID NO: 109 - protein, 485 aa, synthetic construct
  REGION 1..485: note = CD22
MTLDEEYLDITFLTENGFVRKRCPKCGKHFWSADPEREICGDPPCESYSFIGNPAFKNPFELDEMREYYLNFFERRGHGRIERYPVVAWWRTDIYLTIASIADFQPFVTSGVAPPPANPLTISQPCIRLDDLDCVGRTGRHLTLFEMMAHHAFNYPGKEIYWKNETVAYCTELLNELGVKKEDIVYKEELHAGGGNAGPCLEAIVGGLEVAGLGFMNLEEHPEGDIEIKGARYRKMDNYIVDVGYGLERFVWASKGTPTVYDAIFPEVVDTIIDNSNVSFNREDDRVRRIVAESSKLAGIMGELRGERLNRLRKSVADIVGVSVEELEGIVVPLEKVYSLADHTRCILFMLGDGLVPSKAGAGYLARLMIRRSLRLAEELELGLDLYDLVEMHKKILGFEFDVPLSTVQEILELEKERYRTTVSKGTRLVERLVERKKKLEKDDLIELYCSHGIPVALAVGIAAEKGAEVEMPKDIYAELAKRHS

SEQ ID NO: 110 - protein, 383 aa, synthetic construct
  REGION 1..383: note = CD23
MSKSTAEIRQAFLDFFHSKGHQVVASSSLVPHNDPTLLFTNAGMNQFKDVFLGLDKRNYSRATTSQRCVRAGGKHNDLENVGYTARHHTFFEMLGNFSFGDYFKHDAIQFAWELLTSEKWFALPKERLWVTVYESDDEAYEIWEKEVGIPRERIIRIGDNKGAPYASDNFHIMGDTGPCGPCTEIFYDHGDHIWGGPPGSPEEDGDRYIEIWNIGFIQFNRQADGTMEPLPKPSVDTGMGLERIAAVLQHVNSNYDIDLFRTLIQAVAKVTGATDLSNKSLRVIADHIRSCAFLIADGVMPSNENRGYVLRRIIRRAVRHGNMLGAKETFFYKLVGPLIDVMGSAGEDLKRQQAQVEQVLKTEEEQFARTLERGLALLDEELA

SEQ ID NO: 111 - protein, 505 aa, synthetic construct
  REGION 1..505: note = CD24
MTLDEEYLDITFLTENGFVRKRCPKCGKHFWTADPEREICGDPPCESYSFIGNPVFKKPFELDMSKSTAEIRQAFLDFFHSKGHQVVASSSLVPHNDPTLLFTNAGMNQFKDVFLGLDKRNYSRATTSQRCVRAGGKHNDLENVGYTARHHTFFEMLGNFSFGDYFKHDAIQFAWELLTSEKWFALPKERLWVTVYESDDEAYEIWEKEVGIPRERIIRIGDNKGAPYASDNFHQMGDTGPCGPCTEIFYDHGDHIWGGPPGSPEEDGDRYIEIWNIGFMQFNRQADGTMEPLPKPSVDVGMGLERIAAVLQHVNSNYDIDLFRTLIQAVAKVTGATDLSNKSLRVIADHIRSCAFLIADGVMPSKENRGYVLRRIIRRAVRHGNMLGAKETFFYKLVGPLIDVMGSAGEDLKRQQAQVEQVLKTEEEQFARTLERGLALLDEELAKLSGDTLDGETAFRLYRTYGFPVALTADVCRERNIKVDEAGFEAAMEEQRRRAREASGF

SEQ ID NO: 112 - protein, 34 aa, synthetic construct
  MOD_RES 2: note = X is Aib
HXQGTFTSDYSKYLDEKKACEFVEWLLEGGPSSG

SEQ ID NO: 113 - protein, 39 aa, synthetic construct
  MOD_RES 2: note = X is Aib
HXQGTFTSDCSKYLDERAAQDFVQWLLDGGPSSGAPPPS

SEQ ID NO: 114 - protein, 39 aa, synthetic construct
  MOD_RES 2: note = X is Aib
HXQGTFTSDCSKYLDERAAQDFVQWLLEGGPSSGAPPPS

SEQ ID NO: 115 - protein, 34 aa, synthetic construct
  MOD_RES 2: note = x is Aib
HXQGTFTSDCSKYLDERAAQDFVQWLLDGGPSSG

SEQ ID NO: 116 - protein, 68 aa, synthetic construct
  MOD_RES 2: note = X is Aib
HXEGTFTSDVSSYLEEQAAREFIAWLVRGRKGGGGEASELSTAALGRLSAELHELATLPRTETGSGSP

SEQ ID NO: 117 - protein, 76 aa, synthetic construct
  MOD_RES 2: note = X is Aib
YXEGTFTSDYSILLEEQAAREFIEWLLAGGPSSGAGQAPGQAPLASHLSTAQTQRLSAELHKLATLPRTETGSGSP

SEQ ID NO: 118 - DNA, 33 bp, synthetic construct
  misc_feature 1..33: note = ChAlaT-1-F
  source 1..33: mol_type = other DNA
ttcgctaaggatctgcagtggtggagctaccgg

SEQ ID NO: 119 - DNA, 35 bp, synthetic construct
  misc_feature 1..35: note = ChAlaT-1-R
  source 1..35: mol_type = other DNA
ctacatgatcatagctccgaattcagcgttacaag

SEQ ID NO: 120 - DNA, 45 bp, synthetic construct
  misc_feature 1..45: note = ChAlaT-2-F
  source 1..45: mol_type = other DNA
cgctaaggatctgcagtggtggactcgccgggaatctaacccggc

SEQ ID NO: 121 - DNA, 43 bp, synthetic construct
  misc_feature 1..43: note = ChAlaT-2-R
  source 1..43: mol_type = other DNA
cttgtaacgctgaattcggactcgtgatcatgtagatcgaacg

SEQ ID NO: 122 - DNA, 45 bp, synthetic construct
  misc_feature 1..45: note = ChAlaT-3-F
  source 1..45: mol_type = other DNA
ctaaggatctgcagtggtaaggactccgggaatctaacccggctg

SEQ ID NO: 123 - DNA, 46 bp, synthetic construct
  misc_feature 1..46: note = ChAlaT-3-R
  source 1..46: mol_type = other DNA
gtccgttcgatctacatgatcaagcccttgaattcagcgttacaag

SEQ ID NO: 124 - DNA, 47 bp, synthetic construct
  misc_feature 1..47: note = ChAlaT-4-F
  source 1..47: mol_type = other DNA
ctaaggatctgcagtggtggactcgcggggggtcgaaccccgcttac

SEQ ID NO: 125 - DNA, 45 bp, synthetic construct
  misc_feature 1..45: note = ChAlaT-4-R
  source 1..45: mol_type = other DNA
gacccgtttgctcgccggagccgagtccgaattcagcgttacaag

SEQ ID NO: 126 - DNA, 41 bp, synthetic construct
  misc_feature 1..41: note = ChAlaT-5-F
  source 1..41: mol_type = other DNA
cgctaaggatctgcagtggtggaagagccgggaatcgaacc

SEQ ID NO: 127 - DNA, 46 bp, synthetic construct
  misc_feature 1..46: note = ChAlaT-5-R
  source 1..46: mol_type = other DNA
gttcggccaccttggccactcttccgaattcagcgttacaagtatt

SEQ ID NO: 128 - DNA, 41 bp, synthetic construct
  misc_feature 1..41: note = pNEG-V-F
  source 1..41: mol_type = other DNA
gaattcagcgttacaagtattacacaaagttttttatgttg

SEQ ID NO: 129 - DNA, 41 bp, synthetic construct
  misc_feature 1..41: note = pNEG-V-R
  source 1..41: mol_type = other DNA
cttaaaaaaaatccttagctttcgctaaggatctgcagtgg

SEQ ID NO: 130 - DNA, 46 bp, synthetic construct
  misc_feature 1..46: note = Pyl-F1
  source 1..46: mol_type = other DNA
ggcggaagtggtggcggaagcatggataagaagccgctggatgttc

SEQ ID NO: 131 - DNA, 45 bp, synthetic construct
  misc_feature 1..45: note = Pyl-R1
  source 1..45: mol_type = other DNA
gcgtttgaaactgcagttaagaggtcggaaccggagagttcgggg

SEQ ID NO: 132 - DNA, 45 bp, synthetic construct
  misc_feature 1..45: note = Pyl-R2
  source 1..45: mol_type = other DNA
gccgccgcttccgccaccctcgcgttcctcagcgtagatctgttg

SEQ ID NO: 133 - DNA, 42 bp, synthetic construct
  misc_feature 1..42: note = Pyl-F3
  source 1..42: mol_type = other DNA
cgctttgaggaatcccatatggtggtcaagttcaccgacagc

SEQ ID NO: 134 - DNA, 45 bp, synthetic construct
  misc_feature 1..45: note = Pyl-R3
  source 1..45: mol_type = other DNA
cgtgatcgccaatcctcggaacggtggcggaagcggcggcatgac

SEQ ID NO: 135 - DNA, 38 bp, synthetic construct
  misc_feature 1..38: note = Ala-F1
  source 1..38: mol_type = other DNA
cgctttgaggaatcccatatgagcaagagcaccgctga

SEQ ID NO: 136 - DNA, 52 bp, synthetic construct
  misc_feature 1..52: note = Ala-R1
  source 1..52: mol_type = other DNA
cgcttccgccgccgcttccgccacctgccagctcttcatccagcaacgccag

SEQ ID NO: 137 - DNA, 40 bp, synthetic construct
  misc_feature 1..40: note = Ala-F2
  source 1..40: mol_type = other DNA
gtggcggaagcggcggcatgactctcgacgaggagtatct

SEQ ID NO: 138 - DNA, 39 bp, synthetic construct
  misc_feature 1..39: note = Ala-R2
  source 1..39: mol_type = other DNA
gctggcaaagaggcactcataactgcagtttcaaacgct

SEQ ID NO: 139 - DNA, 33 bp, synthetic construct
  misc_feature 1..33: note = pBK-V-F
  source 1..33: mol_type = other DNA
ctgcagtttcaaacgctaaattgcctgatgcgc

SEQ ID NO: 140 - DNA, 33 bp, synthetic construct
  misc_feature 1..33: note = pBK-V-R
  source 1..33: mol_type = other DNA
gttatacgttgtttacgctttgaggaatcccat

SEQ ID NO: 141 - DNA, 46 bp, synthetic construct
  misc_feature 1..46: note = 147-F
  variation 19: note = n = a, c, g or t
  variation 20: note = n = a, c, g or t
  variation 21: note = n = g or t
  source 1..46: mol_type = other DNA
catcttacgcttttcgagnnnatggctcatcatgcgttcaactatc

SEQ ID NO: 142 - DNA, 29 bp, synthetic construct
  misc_feature 1..29: note = 147-R
  source 1..29: mol_type = other DNA
gaacgggaaggcatcttacgcttttcgag

SEQ ID NO: 143 - DNA, 42 bp, synthetic construct
  misc_feature 1..42: note = 191-F
  variation 19: note = n = a, c, g or t
  variation 20: note = n = a, c, g or t
  variation 21: note = n = g or t
  source 1..42: mol_type = other DNA
gtttacaaggaggagccgnnngcgggtgggggtaatgctggc

SEQ ID NO: 144 - DNA, 28 bp, synthetic construct
  misc_feature 1..28: note = 191-R
  source 1..28: mol_type = other DNA
ggaagacattgtttacaaggaggagccg

SEQ ID NO: 145 - DNA, 43 bp, synthetic construct
  misc_feature 1..43: note = 214-F
  variation 19: note = n = a, c, g or t
  variation 20: note = n = a, c, g or t
  variation 21: note = n = g or t
  source 1..43: mol_type = other DNA
cttgaggttgccacgctgnnnttcatgaaccttgaggagcatc

SEQ ID NO: 146 - DNA, 25 bp, synthetic construct
  misc_feature 1..25: note = 214-R
  source 1..25: mol_type = other DNA
cggcggtcttgaggttgccacgctg

SEQ ID NO: 147 - DNA, 30 bp, synthetic construct
  misc_feature 1..30: note = 242/243-F
  source 1..30: mol_type = other DNA
ggctacggtcttgaaaggttcgtgtgggcg

SEQ ID NO: 148 - DNA, 44 bp, synthetic construct
  misc_feature 1..44: note = 242/243-R
  variation 26: note = n = a, c, g or t
  variation 27: note = n = a, c, g or t
  variation 28: note = n = g or t
  source 1..44: mol_type = other DNA
cagaaaaatggacaactacatcgttnnnactggctacggtcttg

SEQ ID NO: 149 - DNA, 45 bp, synthetic construct
  misc_feature 1..45: note = 212/214-F
  variation 15: note = n = a, c, g or t
  variation 16: note = n = a, c, g or t
  variation 17: note = n = g or t
  variation 21: note = n = a, c, g or t
  variation 22: note = n = a, c, g or t
  variation 23: note = n = g or t
  source 1..45: mol_type = other DNA
gtcttgaggttgccnnnctgnnnttcatgaaccttgaggagcatc

SEQ ID NO: 150 - DNA, 30 bp, synthetic construct
  misc_feature 1..30: note = 212/214-R
  source 1..30: mol_type = other DNA
ggcaacctcaagaccgccgactatggcctc

SEQ ID NO: 151 - protein, 22 aa, synthetic construct
  REGION 1..22: note = pelb
MKYLLPTAAAGLLLLAAQPAMA

SEQ ID NO: 152 - protein, 26 aa, synthetic construct
  REGION 1..26: note = Mae
MKIKTGARILALSALTTMMFSASALA

SEQ ID NO: 153 - protein, 19 aa, synthetic construct
  REGION 1..19: note = DsbA
MKKIWLALAGLVLAFSASA

SEQ ID NO: 154 - RNA, 72 nt, synthetic construct
  source 1..72: mol_type = other RNA
ggagctatgatcatgtagatcgaacggactctaaatccgttcagccgggttagattcccggtagctccacca

SEQ ID NO: 155 - RNA, 72 nt, synthetic construct
  source 1..72: mol_type = other RNA
ggactcgtgatcatgtagatcgaacggactctaaatccgttcagccgggttagattcccggcgagtccacca